(19) United States
(12) Patent Application Publication
(10) Pub. No.: US 2024/0262869 A1
(43) Pub. Date: Aug. 8, 2024

(54) POLYPEPTIDE FRAGMENTS, IMMUNOGENIC COMPOSITION AGAINST SARS-CoV-2, AND IMPLEMENTATIONS THEREOF

(71) Applicants: Indian Institute of Science, Bangalore (IN); MYNVAX PRIVATE LIMITED, Bangalore (IN)

(72) Inventors: Raghavan VARADARAJAN, Bangalore (IN); Sameer Kumar MALLADI, Bangalore (IN); Shahbaz AHMED, Bangalore (IN); Suman PANDEY, East Bangalore (IN); Randhir SINGH, Bangalore (IN)

(73) Assignees: Indian Institute of Science, Bangalore (IN); MYNVAX PRIVATE LIMITED, Bangalore (IN)

(21) Appl. No.: 18/004,065

(22) PCT Filed: Jun. 29, 2021
(86) PCT No.: PCT/IN2021/050631

§ 371 Date: Jan. 2, 2023

(30) Foreign Application Priority Data
Jul. 3, 2020 (IN) 202041028428

(51) Int. Cl. C07K 14/005; A61K 9/00; A61K 39/215; A61P 31/14

(52) U.S. Cl. CPC C07K 14/005; A61K 9/0019; A61K 39/215; A61P 31/14; C12N 2770/20022; C12N 2770/20034; C12N 2770/20071

(57) ABSTRACT

The present disclosure discloses the polypeptide fragment having an amino acid sequence with at least 95% identity to the amino acid sequence selected from the group consisting of SEQ ID NO: 2, SEQ ID NO: 4, and SEQ ID NO: 6. The present disclosure also discloses nucleic acid fragment encoding the polypeptide fragment as described herein. Moreover, the present disclosure also discloses recombinant construct, recombinant vector and recombinant host cells. Also disclosed herein is an immunogenic composition comprising the polypeptide fragment as described herein, and a method for preparing the said immunogenic composition. The immunogenic composition is in form of vaccine. The polypeptide fragment and/or immunogenic composition is capable of eliciting protection against severe acute respiratory syndrome coronavirus 2. A kit comprising the polypeptide, or the immunogenic composition as described herein is also disclosed.

### FIELD OF INVENTION

The present disclosure broadly relates to the field of immunobiology, and particularly discloses immunogenic polypeptides, and immunogenic composition for eliciting immune response against sever acute respiratory syndrome coronavirus 2 (SARS-CoV-2).

### BACKGROUND OF THE INVENTION

The Coronavirus infectious disease 2019 (COVID-19) pandemic caused by SARS-CoV-2 has led to approximately 141.7 million infections and approximately 3.0 million deaths worldwide as on 2 Apr. 2021 (J. Shang, et al., Cell entry mechanisms of SARS-CoV-2. Proc. Natl. Acad. Sci. 117, 11727-11734 (2020). India is currently in the throes of a debilitating second wave, with the highest daily infection rate in the world. The viral spike glycoprotein is the most abundant protein exposed on the viral surface and the primary target of host elicited humoral immune responses. Spike glycoprotein, like various class I viral surface glycoproteins, assembles as a trimer with each protomer composed of the surface exposed S1 and membrane anchored S2 subunit (L. Dai, et al., A Universal Design of Betacoronavirus Vaccines against COVID-19, MERS, and SARS. Cell 182, 722-733.e11 (2020)). The S1 subunit consists of four independently folding domains: N-terminal domain (NTD), receptor binding domain (RBD), and two short domains (SD1 and SD2) connected by linker regions (P. J. M. Brouwer, et al., Potent neutralizing antibodies from COVID-19 patients define multiple targets of vulnerability. Science 369, 643-650 (2020)). The receptor binding domain (RBD) contains the receptor binding motif (residues 438-505) that facilitates interaction with the angiotensin-converting enzyme 2 (ACE2) receptor. The subsequent fusion or endocytosis is mediated by the fusion peptide that constitutes the N-terminal stretch of the S2 subunit (L. Dai, et al., A Universal Design of Betacoronavirus Vaccines against COVID-19, MERS, and SARS. Cell 182, 722-733.e11 (2020)). Hence, it can be concluded that the majority of neutralizing antibodies in both natural infection and vaccination target the RBD.

Multiple efforts have been made for creating various vaccines for coronavirus infections. The developed vaccine candidates can be divided into six classes: 1) viral-vector vaccines; 2) DNA vaccines; 3) subunit vaccines; 4) nano-particles-based vaccines; 5) inactivated whole-virus vaccines; and 6) live attenuated vaccines.

For instance, the U.S. Pat. No. 7,452,542B2 discloses a live, attenuated coronavirus vaccines. The vaccine comprises a viral genome encoding a p59 protein having at mutation at a specific tyrosine residue and may include other attenuating mutations. Such viruses show reduced growth and pathogenicity in-vivo.

The Patent Application WO2016116398A1 relates to the Middle East Respiratory Syndrome Coronavirus (MERS-CoV) N nucleocapsid protein and/or an immunogenic fragment thereof, or a nucleic acid molecule encoding the MERS-CoV N nucleocapsid protein and/or the immunogenic fragment thereof, for use as a vaccine.

Currently, there are a large number of COVID-19 vaccine candidates in various stages of development, with approximately 11 candidates already granted emergency use authorisation. In addition, there are recent reports of new strains of the virus with enhanced transmissibility and immune evasion (J. Yang, et al., A vaccine targeting the RBD of the S protein of SARS-CoV-2 induces protective immunity. Nature 586, 572-577 (2020)).

Since the current vaccine formulations available in the literature are required to be stored either refrigerated or frozen, and are also not very effective against mutation in viral sequences, therefore, there is a dire need to develop safe, cheap and efficacious vaccine that can be stored for extended periods at room temperature and also elicit high titers of neutralizing antibodies to buffer against viral sequence variation, in order to protect those most in need, worldwide.

### SUMMARY OF INVENTION

In an aspect of the present disclosure, there is provided a polypeptide fragment having an amino acid sequence with at least 95% identity to the amino acid sequence selected from the group consisting of SEQ ID NO: 2, SEQ ID NO: 4, SEQ ID NO: 6.

In another aspect of the present disclosure, there is provided a polypeptide fragment comprising: (a) a polypeptide having an amino acid sequence selected from the group consisting of SEQ ID NO: 2; or SEQ ID NO: 8; (b) a polypeptide having a substitution at an amino acid position in SEQ ID NO: 2, wherein the substitution at the amino acid position is selected from the group consisting of positions at 3, 7, 16, 18, 24, 28, 35, 37, 39, 42, 43, 53, 55, 59, 60, 62, 78, 84, 98, 100, 104, 129, 130, 134, 138, 147, 190, and 197; (c) a polypeptide having a substitution at an amino acid position in SEQ ID NO: 8, wherein the substitution at the amino acid position is selected from the group consisting of positions at 6, 10, 19, 21, 27, 31, 38, 40, 42, 45, 46, 56, 58, 62, 63, 65, 81, 87, 101, 103, 107, 132, 133, 137, 141, 150, 193, and 200; (d) a polypeptide having at least one variation in the amino acid sequence as set forth in SEQ ID NO: 2, wherein the at least one variation is selected from the group consisting of P197R/K198R/K199V/S200P/N202V, P197L/Y35F, P197L/A190G/Y35F, P197L/A190G/Y35F/T3H, P197L/A190G/Y35F/T 3H/T55S, P197L/A190G/Y35F, P197L/A190G/Y35F/T3H/T55S/V173D, A18P/P197L/A190G/Y35F/T3H, A18P/A42M/P197L/A190G/Y35F/T3H, A18P/A42M/T100V/P197L/A190G/Y35F/T3H, Y35W/L60M/N118D/Q163S/C195D, A18P/Y35W/P197L, A18P/V37F/P197L, A18P/Y35W/V37F/P197L, A18P/V37F/P197I, A18P/Y35W/V37F/P197I, N13D/A18P/V37F/P197L, N13D/A18P/Y35W/P197L, I28F/Y35W, I28F/F62W, I28F/I104F, Y35W/Y62W, Y35W/I104F, Y62W/I104F, I28F/Y35W/F62W, I28F/Y35W/I104F, I28F/F62W/I104F, Y35W/F62W/I104F, or I28F/Y35W/F62W/I104F; (e) a polypeptide having at least one variation in the amino acid sequence as set forth in SEQ ID NO: 8, wherein the at least one variation is selected from the group consisting of P200R/K201R/K202V/S203P/N205V, P200L/Y38F, P200L/A193G/Y38F, P200L/A193G/Y38F/T6H, P200L/A193G/Y38F/T6H/T58S, P200L/A193G/Y38F/T6H/T58S/V176D, A21P/P200L/A193G/Y38F/T6H, A21P/A45M/P200L/A193G/Y38F/T6H, A21P/A45M/T103V/P200L/A193G/Y38F/T6H, Y38W/L63M/N121D/Q166S/C198D, A21P/Y38W/P200L, A21P/V40F/P200L, A21P/Y38W/V40F/P200L, A21P/V40F/P200I, A21P/Y38W/V40F/P200I, N16D/A21P/V40F/P200L, N16D/A21P/Y38W/P200L, I31F/Y37W, I31F/F65W, I31F/I107F, Y38W/Y65W, Y38W/I107F, Y65W/I107F, I31F/Y38W/F65W, I31F/Y38W/I107F, I31F/F65W/I107F, Y38W/F65W/I107F, or I31F/Y38W/F65W/I107F; or (f) a polypeptide having an amino acid selected from the group having the amino acid sequence as set forth in SEQ ID NO: 76, and SEQ ID NO: 79.

In another aspect of the present disclosure, there is provided a polypeptide fragment comprising: (a) a polypeptide having an amino acid sequence selected from the group consisting of SEQ ID NO: 4, or SEQ ID NO: 10; (b) a polypeptide having a substitution at an amino acid position in SEQ ID NO: 4, wherein the substitution at the amino acid position is selected from the group consisting of positions at 2, 6, 15, 17, 23, 27, 34, 36, 38, 41, 42, 52, 54, 58, 59, 61, 77, 83, 97, 99, 103, 128, 129, 133, 137, 146, 189, and 196; (c) a polypeptide having a substitution at an amino acid position in SEQ ID NO: 10, wherein the substitution at the amino acid position is selected from the group consisting of positions at 5, 9, 18, 20, 26, 30, 37, 39, 41, 44, 45, 55, 57, 61, 62, 64, 80, 86, 100, 102, 106, 131, 132, 136, 140, 149, 192, and 199; (d) a polypeptide having at least one variation in the amino acid sequence as set forth in SEQ ID NO: 4, wherein the at least one variation is selected from the group consisting of P196R/K197R/K198V/S199P/N201V, P196L/Y34F, P196L/A189G/Y34F, P196L/A189G/Y34F/T2H, P196L/A189G/Y34F/T2H/T54S, P196L/A189G/Y34F/T2H/T54S/V172D, A17P/P196L/A189G/Y34F/T2H, A17P/A41M/P196L/A189G/Y34F/T2H, A17P/A41M/T99V/P196L/A189G/Y34F/T2H, Y34W/L59M/N117D/Q162S/C194D, A17P/Y34W/P196, A17P/V36F/P196L, A17P/Y34W/V36F/P196L, A17P/V36F/P196I, A17P/Y34W/V36F/P196I, N12D/A17P/V36F/P196L, N12D/A17P/Y34W/P196L, I27F/Y34W, I27F/F61W, I27F/I103F, Y34W/Y61W, Y34W/I103F, Y61W/I103F, I27F/Y34W/F61W, I27F/Y34W/I103F, I27F/F61W/I102F, Y34W/F61W/I103F, I27F/Y34W/F62W/I103F; (e) a polypeptide having at least one variation in the amino acid sequence as set forth in SEQ ID NO: 10, wherein the at least one variation is selected from the group consisting of P199R/K200R/K201V/S202P/N204V, P199L/Y37F, P199L/A192G/Y37F, P199L/A192G/Y37F/T5H, P199L/A192G/Y 37F/T5H/T57S, P199L/A192G/Y37F/T5H/T57S/V175D, A20P/P199L/A192G/Y37F/T5H, A20P/A44M/P199L/A192G/Y37F/T5H, A20P/A44M/T102V/P199L/A192G/Y37F/T5H, Y37W/L62M/N120D/Q165S/C197D, A20P/Y37W/P199L, A20P/V39F/P199L, A20P/Y37W/V39F/P199L, A20P/V39F/P199I, A20P/Y37W/V39F/P199I, N15D/A20P/V39F/P199L, N15D/A20P/Y37W/P199L, I30F/Y36W, I30F/F64W, I30F/I106F, Y37W/Y64W, Y37W/I106F, Y64W/I106F, I30F/Y37W/F64W, I30F/Y37W/I106F, I30F/F65W/I106F, Y37W/F64W/I106F, I30F/Y37W/F64W/I106F; or (f) a polypeptide having an amino acid as set forth in SEQ ID NO: 77.

In another aspect of the present disclosure, there is provided a polypeptide fragment comprising: (a) a polypeptide having an amino acid sequence selected from the group consisting of SEQ ID NO: 6, or SEQ ID NO: 12; (b) a polypeptide having a substitution at an amino acid position in SEQ ID NO: 6, wherein the substitution at the amino acid position is selected from the group consisting of positions at 2, 6, 15, 17, 23, 27, 34, 36, 38, 41, 42, 52, 54, 58, 59, 61, 77, 83, 97, 99, 103, 128, 129, 133, 137, 146, 189, and 196; (c) a polypeptide having a substitution at an amino acid position in SEQ ID NO: 12, wherein the substitution at the amino acid position is selected from the group consisting of positions at 5, 9, 18, 20, 26, 30, 37, 39, 41, 44, 45, 55, 57, 61, 62, 64, 80, 86, 100, 102, 106, 110, 131, 132, 136, 140, 149, 192, and 199; (d) a polypeptide having at least one variation in the amino acid sequence as set forth in SEQ ID NO: 6, wherein the at least one variation is selected from the group consisting of P196R/K197R/K198V/S199P/N201V, P196L/Y34F, P196L/A189G/Y34F, P196L/A189G/Y34F/T2H, P196L/A189G/Y34F/T2H/T54S, P196L/A189G/Y34F/T2H/T54S/V172D, A17P/P196L/A189G/Y34F/T2H, A17P/A41M/P196L/A189G/Y34F/T2H, A17P/A41M/T99V/P196L/A189G/Y34F/T2H, Y34W/L59M/N117D/Q162S/C194D, A17P/Y34W/P196L, A17P/V36F/P196L, A17P/Y34W/V36F/P196L, A17P/V36F/P196I, A17P/Y34W/V36F/P196I, N12D/A17P/V36F/P196L, N12D/A17P/Y34W/P196L, I27F/Y34W, I27F/F61W, I27F/I103F, Y34W/Y61W, Y34W/I103F, Y61W/I103F, I27F/Y34W/F61W, I27F/Y34W/I103F, I27F/F61W/I102F, Y34W/F61W/I103F, and I27F/Y34W/F62W/I103F; (e) a polypeptide having at least one variation in the amino acid sequence as set forth in SEQ ID NO: 12, wherein the at least one variation is selected from the group consisting of P199R/K200R/K201V/S202P/N204V, P199L/Y37F, P199L/A192G/Y37F, P199L/A192G/Y37F/T5H, P199L/A192G/Y 37F/T5H/T57S, P199L/A192G/Y37F/T5H/T57S/V175D, A20P/P199L/A192G/Y37F/T5H, A20P/A44M/P199L/A192G/Y37F/T5H, A20P/A44M/T102V/P199L/A192G/Y37F/T5H, Y37W/L62M/N120D/Q165S/C197D, A20P/Y37W/P199L, A20P/V39F/P199L, A20P/Y37W/V39F/P199L, A20P/V39F/P199I, A20P/Y37W/V39F/P199I, N15D/A20P/V39F/P199L, N15D/A20P/Y37W/P199L, I30F/Y36W, I30F/F64W, I30F/I106F, Y37W/Y64W, Y37W/I106F, Y64W/I106F, I30F/Y37W/F64W, I30F/Y37W/I106F, I30F/F65W/I106F, Y37W/F64W/I106F, and I30F/Y37W/F64W/I106F; or (f) a polypeptide having an amino acid selected from the group having the amino acid sequence as set forth in SEQ ID NO: 69, SEQ ID NO: 70, SEQ ID NO: 71, SEQ ID NO: 72, SEQ ID NO: 73, SEQ ID NO: 74, and SEQ ID NO: 85.

In another aspect of the present disclosure, there is provided a polypeptide fragment comprising: (a) a polypeptide having an amino acid sequence selected from the group consisting of SEQ ID NO:14, SEQ ID NO: 16, SEQ ID NO: 18, SEQ ID NO: 20, and SEQ ID NO: 22; or (b) a polypeptide having an amino acid sequence selected from the group consisting of SEQ ID NO: 81, and SEQ ID NO: 83.

In another aspect of the present disclosure, there is provided a polypeptide fragment comprising: (a) a polypeptide having an amino acid sequence selected from the group consisting of SEQ ID NO: 56, SEQ ID NO: 58, and SEQ ID NO: 60; (b) a polypeptide having an amino acid sequence selected from the group consisting of SEQ ID NO: 52, SEQ ID NO: 54, SEQ ID NO: 62, SEQ ID NO: 64, SEQ ID NO: 66, and SEQ ID NO: 68; or (c) a polypeptide having an amino acid sequence selected from the group consisting of SEQ ID NO: 24, SEQ ID NO: 26, SEQ ID NO: 28, SEQ ID NO: 30, SEQ ID NO: 32, SEQ ID NO: 34, SEQ ID NO: 38, SEQ ID NO: 40, SEQ ID NO: 42, SEQ ID NO: 44, SEQ ID NO: 46, SEQ ID NO: 48, and SEQ ID NO: 50.

In another aspect of the present disclosure, there is provided a polypeptide fragment comprising a polypeptide having an amino acid sequence selected from the group consisting of SEQ ID NO: 8, SEQ ID NO: 10, SEQ ID NO: 12, SEQ ID NO: 14, SEQ ID NO: 69, SEQ ID NO: 70, SEQ ID NO: 71, SEQ ID NO: 73, SEQ ID NO: 74, SEQ ID NO: 76, SEQ ID NO: 77, SEQ ID NO: 79, SEQ ID NO: 81, SEQ ID NO: 83, and SEQ ID NO: 85.

In another aspect of the present disclosure, there is provided a recombinant construct comprising the nucleic acid fragment, said nucleic acid fragment encoding a polypeptide fragment as described herein, operably linked to a promoter.

In another aspect of the present disclosure, there is provided a recombinant vector comprising the recombinant construct as described herein.

In another aspect of the present disclosure, there is provided a recombinant host cell comprising the recombinant construct as described herein or the recombinant vector as described herein.

In another aspect of the present disclosure, there is provided an immunogenic composition comprising a polypeptide fragment as described herein and a pharmaceutically acceptable carrier.

In another aspect of the present disclosure, there is provided an immunogenic composition comprising: (a) a combination of at least two polypeptide fragments having an amino acid sequence selected from the group consisting of SEQ ID NO: 69, and SEQ ID No: 78, and a pharmaceutical acceptable carrier; (b) a combination of at least two polypeptide fragments having an amino acid sequence selected from the group consisting of SEQ ID NO: 81, and SEQ ID NO: 83, and a pharmaceutically acceptable carrier.

In another aspect of the present disclosure, there is provided a method for obtaining the immunogenic composition as described herein, wherein the method comprises: (a) culturing the recombinant host cell as described herein under suitable conditions to obtain the polypeptide as described herein; (b) subjecting the polypeptide to purification; and (c) contacting the polypeptide of step (b) with a pharmaceutically acceptable carrier for obtaining the immunogenic composition.

In another aspect of the present disclosure, there is provided a method for eliciting an immune response in a subject, the method comprising administering the subject a pharmaceutically effective amount of the immunogenic composition as described herein.

In another aspect of the present disclosure, there is provided a kit comprising the polypeptide as described herein or the immunogenic composition as described herein, and an instruction leaflet.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts S-protein domain organization, structure of Spike and receptor binding domain of SARS-CoV-2. A) Linear map of the S protein spike with the following domains: NTD, N-terminal domain; L, linker region; RBD, receptor-binding domain; SD, subdomain; UH, upstream helix; FP, fusion peptide; CR, connecting region; HR, heptad repeat; CH, central helix; BH, β-hairpin; TM, transmembrane region/domain; CT, cytoplasmic tail. B) Spike ecto domain trimer highlighting protomer with RBD in up conformation, NTD in dark blue, RBD in brick red, SD1 and SD2 in green and S2 subunit in megenta (PDB: 6VSB) C) One RBD derivative being utilized as an vaccine candidate (residues 332-532) with the cystine pairs highlighted in green, glycosylation site highlighted in blue and the receptor binding motif highlighted in orange (PDB: 6M0J), in accordance with an embodiment of the present disclosure.

FIG. 2 depicts mInCV02R purification and thermal stability. A) Size exclusion chromatography profile of mInCV02R vaccine candidate (SEQ ID NO: 10) with predominantly monomeric peak at ˜16.3 ml on S200 10/300GL column run at flowrate of 0.5 ml/min with PBS (pH 7.4) as mobile phase. B) Coomassie stained mInCV02R purified from Expi293F cells incubated at various temperatures D-Dialysed and stored overnight at 4° C., 4—4° C. stored protein, −80—80° C. frozen and thawed protein, 37—protein incubated at 37° C. for 1 hour without glycerol, 37G—protein incubated at 37° C. for 1 hour with 5% glycerol, L—Biorad ladder (Cat No: #161-0374) C) Coomassie stained mInCV02R protein in the presence and absence of reducing agent DTT. D) Limited proteolysis of purified mInCV02R protein by TPCK treated trypsin (RBD:TPCK Trypsin=50:1) at 4° C. and 37° C. The protein is protected for ˜60 and ˜30 minutes at 4° C. and 37° C. respectively E) nanoDSF equilibrium thermal unfolding of mInCV02R, in accordance with an embodiment of the present disclosure.

FIG. 3 depicts mInCV02R aggregation profile upon thermal stress and freeze thaw. Size exclusion chromatography profile of mInCV02R vaccine candidate (SEQ ID NO: 10) A) dialyzed and stored over night at 4° C. C) stored at 37° C. for 1 hour D) frozen at −80° C. and thawed, displays a predominantly monomeric peak at ˜16.3 ml on S200 10/300GL column run at flowrate of 0.5 ml/min with PBS (pH 7.4) as mobile phase. B) Reducing SDS-PAGE coomassie stained mInCV02R purified from Expi293F cells incubated at various temperatures D—Dialysed and stored overnight at 4° C., 4—4° C. stored protein, −80—80° C. frozen and thawed protein, 37—protein incubated at 37° C. for 1 hour without glycerol, 37G—protein incubated at 37° C. for 1 hour with 5% glycerol, L—Biorad ladder (Cat No: #161-0374), in accordance with an embodiment of the present disclosure.

FIG. 4 depicts mInCV01R purification and thermal stability. A) Size exclusion chromatography profile of mInCV01R vaccine candidate (SEQ ID NO: 8) with predominantly monomeric peak at ˜16.0 ml on S200 10/300GL column run at flowrate of 0.5 ml/min with PBS (pH 7.4) as mobile phase. B) Reducing SDS-PAGE Coomassie stained mInCV01R purified from Expi293F cells incubated at various temperatures, 4—4° C. stored protein, −80, −80° C. frozen and thawed protein, 37—protein incubated at 37° C. for 1 hour without glycerol, 37G—protein incubated at 37° C. for 1 hour with 5% glycerol, L—Biorad ladder (Cat No: #161-0374) C) nanoDSF equilibrium thermal unfolding of mInCV01R D) Coomassie stained purified vaccine candidates under reducing and non-reducing conditions E) Limited proteolysis of purified mInCV01R protein by TPCK treated trypsin (RBD:TPCK Trypsin=50:1) at 4° C. and 37° C. The protein is protected for ˜30 and ˜5 minutes at 4° C. and 37° C. respectively, in accordance with an embodiment of the present disclosure.

FIG. 5 depicts nanoDSF thermal melt of purified vaccine candidates A) mInCV01R (SEQ ID NO: 8), B) mInCV02R (SEQ ID NO: 10) expressed in Expi293F mammalian cells C) iInCV01R (SEQ ID NO: 56), D) iInCV02R (SEQ ID NO: 58) expressed in ExpiSf9 insect cells D) pInCV02R (SEQ ID NO: 60) expressed in Pichia pastoris, in accordance with an embodiment of the present disclosure.

FIG. 6 depicts nanoDSF thermal melt of purified vaccine candidates following affinity tag removal through HRV3C digestion A) mInCV01R (SEQ ID NO: 8); B) mInCV02R (SEQ ID NO: 10) expressed in Expi293F mammalian cells, in accordance with an embodiment of the present disclosure.

FIG. 7 depicts Surface plasmon resonance (SPR) binding sensorgrams to soluble ACE2 receptor (A, B, E) and neutralizing antibody CR3022 (C, D) of purified vaccine candidates A), C) mInCV02R (SEQ ID NO: 10) expressed in Expi293F B), D) pInCV02R (SEQ ID NO: 60) expressed in Pichia E) mInCV21R (SEQ ID NO: 14) expressed in Expi293F cells. The concentrations of mInCV02R and pInCV02R used as analytes are A) 100 nM, 50 nM, 25 nM, 12.5 nM, 6.25 nM B) 100 nM, 50 nM, 25 nM C) 50 nM, 25 nM, 12.5 nM, 6.2 nM, 3.1 nM D) 12.5 nM, 6.2 nM, 3.1 nM. Proteins purified from Expi293F and Pichia bound similarly to ACE2 and CR3022. The designed nanoparticulate vaccine candidate mInCV21R has negligible dissociation upon binding to ACE2 in this SPR format, in accordance with an embodiment of the present disclosure.

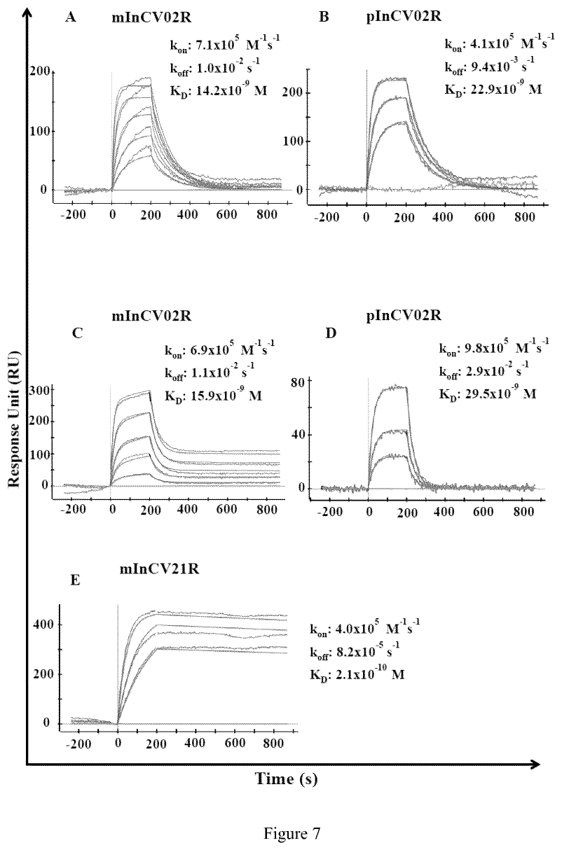

FIG. 8 depicts mInCV05NR, mInCVO7N purification and SPR binding to ACE2 receptor. Size exclusion chromatography profile of A) mInCV05NR (NTD-RBD fusion; SEQ ID NO: 62) and B) mInCVO7N (NTD alone; SEQ ID NO: 64) vaccine candidates show predominantly monomeric peaks at ˜13.3 ml for A) mInCV05NR and ˜15.2 ml for B) mInCVO7N on a S200 10/300GL column run at flowrate of 0.5 ml/min with PBS (pH 7.4) as mobile phase. C) Coomassie stained mInCV05NR and mInCVO7N purified from Expi293F, L—Biorad ladder (Cat No: #161-0374). The black arrow indicates the mInCV05NR and red arrow indicates mInCVO7N D) Surface plasmon resonance (SPR) binding sensorgrams to ACE2 receptor of purified mInCV05NR. Binding to ACE2 confirms the proper folding of the designed NTD-RBD vaccine candidate, in accordance with an embodiment of the present disclosure.

FIG. 9 depicts Surface plasmon resonance (SPR) binding sensorgrams to ACE2 receptor of purified mInCV02R (SEQ ID NO: 10) stored under different conditions. A) incubated at 4° C. overnight B) lyophilized and resuspended in water prior to injection C) incubated at 37° C. overnight without glycerol D) incubated at 37° C. overnight with glycerol. Proteins stored under all these conditions bound similarly to Ace2 with a K_{d} of about 3 nM, in accordance with an embodiment of the present disclosure.

FIG. 10 depicts Surface plasmon resonance (SPR) binding sensograms to macaque ACE2 receptor of purified vaccine candidates from A), B) Expi293F and C), D) ExpiSf9 Proteins from different expression systems bound with similar affinity to macaque ACE2 with a K_{d} of about 3 nM. The concentrations of analytes used are 100 nM, 50 nM, 25 nM, 12.5 nM and 6.25 nM from highest to lowest, in accordance with an embodiment of the present disclosure.

FIG. 11 depicts pInCV02R purification, thermal stability and SPR binding to macaque ACE2 and CR3022 A) Size exclusion chromatography profile of pInCV02R (SEQ ID NO: 60) vaccine candidate with predominantly monomeric peak at ˜14.5 ml on S200 10/300GL column run at flowrate of 0.75 ml/min with PBS (pH 7.4) as mobile phase. B) nanoDSF equilibrium thermal unfolding of pInCV02R. C), D) Surface plasmon resonance (SPR) binding sensograms to macaque ACE2 receptor (C) and neutralizing antibody CR3022 (D) of purified pInCV02R vaccine candidate with concentration of analytes as C) 100 nM, 50 nM, 25 nM D) 12.5 nM, 6.2 nM, 3.1 nM from highest to lowest respectively. E) Limited proteolysis of purified pInCV02R protein by TPCK treated trypsin (RBD:TPCK Trypsin=50:1) at 4° C. and 37° C., in accordance with an embodiment of the present disclosure.

FIG. 12 depicts the arrangement of one of the vaccine candidates which represents RBD chimera fused with SARS-CoV-2 RBD, the RBD chimera consists of Residues 318-442 and 490-518 from SARS-CoV-1 with an insertion of the Receptor Binding Motif (RBM) of SARS-CoV-2 (residues 454-503 of SARS-CoV-2) inserted between residues 442 and 490 of SARS-CoV-1, in accordance with an embodiment of the present disclosure.

FIG. 13 depicts the FACS histogram overlays of binding of putatively stabilized CV01R mutants with Ace-2 (probed 50 nM Ace2), in accordance with an embodiment of the present disclosure.

FIG. 14 depicts the thermal stabilities of WT and stabilized CV01R mutants in PBS buffer estimated using DSF, in accordance with an embodiment of the present disclosure.

FIG. 15 depicts the design and characterization of trimeric RBD. A) The design utilized the RBD (residues 332-532) from the closed state of the Spike-2P (PDB 6VXX) aligned coaxially with the hCMP trimerization domain, coordinates taken from the homolog CCMP (PDB:1AQ5, Chain 1.1). The N termini of mRBD are labelled as 1332 and the hCMP trimerization domain C-termini are labelled as V340. The N, C termini Cα's form vertices of equilateral triangles. The N-terminal plane of RBD (I332) is separated from the C-terminal plane (V340) of the hCMP trimerization domain by ˜22.1 Å to avoid steric clashes. The 1332 terminus and V340 terminus are ˜39 Å apart in the modelled structure and are connected by a 14-residue long linker. B) hCMP-mRBD consists of N-terminal hCMP trimerization domain fused to 1332 of RBD by a linker (L14). mRBD-hCMP consists of the C-terminal hCMP trimerization domain fused to N532 of RBD by a linker (L5). mRBD-GlyIZ consists of a C-terminal GlyIZ trimerization domain fused to N532 of RBD by a linker (L5). MsDPS2-mRBD consists of the MsDPS2 nanoparticle fused to SpyTag covalently linked with mRBD-SpyCatcher. C) SEC elution profile of trimeric hCMP-mRBD. D) SDS-PAGE of purified mRBD and hCMP-mRBD in reducing and non-reducing conditions demonstrating formation of disulfide-linked trimers. E) SEC-MALS of purified hCMP-mRBD (MW: 110±10 kDa). The red, black and blue profiles are of the molar mass fit, molar mass and refractive index (RI) respectively. F) nanoDSF equilibrium thermal unfolding of hCMP-mRBD. G) SDS-PAGE of purified mRBD-GlyIZ and mRBD-hCMP in reducing conditions; H) SEC elution profiles of mRBD-hCMP; and SEC elution profiles of mRBD-GlyIZ; J) SDS-PAGE of purified MsDpS2-SpyTag, mRBD-SpyCatcher and the resulting MsDPS2-SpyTag-mRBD-SpyCatcher conjugate abbreviated MsDPS2-mRBD for simplicity. The black solid line, triangle without fill and red triangle correspond to MsDPS2-SpyTag nanoparticle, mRDS-SpyCatcher and MsDPS2-mRBD conjugate respectively; K) SPR binding of hCMP-mRBD (SEQ ID NO: 14), mRBD-hCMP (SEQ ID NO: 16), mRBD-GlyIZ (SEQ ID NO: 22) and SEC purified complex MsDPS2-mRBD to immobilized ACE2. The curves from highest to lowest correspond to concentrations 100 nM, 50 nM, 25 nM, 12.5 nM and 6.25 nM respectively for hCMP-mRBD, mRBD-hCMP and mRBD-GlyIZ. The curves for MsDPS2-mRBD correspond from highest to lowest concentrations of 10 nM, 5 nM, 2.5 nM and 1.25 nM respectively. ND* denotes no dissociation, in accordance with an embodiment of the present disclosure.

FIG. 16 depicts negative staining TEM analysis of hCMP-mRBD (SEQ ID NO: 14). A) A representative negative staining image of hCMP-mRBD protein. B) Representative reference free 2D class averages of hCMP-mRBD, wherein 2D class averages indicate that hCMP-mRBD protein is monodisperse and stable. The protein forms a stable trimer. The bottom panel shows the enlarged view of class 1 and 7, trimeric hCMP-mRBD protein. C) The reference free 2D classification calculation using SIMPLE 2.1, in accordance with an embodiment of the present disclosure.

FIG. 17 depicts SPR binding of trimeric and nanoparticle RBD to CR3022. SPR binding studies were performed with hCMP-mRBD (SEQ ID NO: 14), mRBD-hCMP (SEQ ID NO: 16), mRBD-GlyIZ (SEQ ID NO: 22) and SEC purified complex MsDPS2-mRBD to CR3022. The curves from highest to lowest correspond to concentrations 100 nM, 50 nM, 25 nM, 12.5 nM and 6.25 nM respectively for hCMP-mRBD, mRBD-hCMP and mRBD-GlyIZ. The curves for MsDPS2-mRBD correspond from highest to lowest to concentrations 10 nM, 5 nM, 2.5 nM, and 1.25 nM respectively. ND*—No dissociation, in accordance with an embodiment of the present disclosure.

FIG. 18 depicts characterization of trimeric hCMP-mRBD (SEQ ID NO: 14) following transient exposure to elevated temperature and extended incubation at 37° C. A) hCMP-mRBD in PBS at a concentration of 0.2 mg/ml was subjected to transient thermal stress for one hour and binding studies performed at 100 nM. B) Lyophilized hCMP-mRBD was subjected to transient thermal stress for 90 minutes followed by reconstitution in water. C.) hCMP-mRBD (0.2 mg/ml) in solution subjected to 37° C. incubation as a function of time (3-72 hr) D) Lyophilized hCMP-mRBD subjected to extended thermal stress at 4° C. and 37° C. for 2 and 4 weeks. 100 nM of hCMP-mRBD was used as analyte. (E-F Equilibrium thermal unfolding monitored by nanoDSF of hCMP-mRBD (0.2 mg/ml) subjected to 37° C. incubation in 1×PBS for upto 72 hours. F) Equilibrium thermal unfolding monitored by nanoDSF of lyophilized hCMP-mRBD incubated for upto 4 weeks at 4° C. and 37° C. The lyophilized protein was reconstituted in MilliQ grade water prior to thermal melt and SPR binding studies. The binding to ACE2-hFc was performed at 100 nM. ACE2-hFc immobilized was 800RU, in accordance with an embodiment of the present disclosure.

FIG. 19 depicts characterization of mRBD-GlyIZ (SEQ ID NO: 22) trimeric RBD following transient exposure to elevated temperature. A) mRBD-GlyIZ in PBS at a concentration of 0.2 mg/ml was subjected to transient thermal stress for one hour and binding studies performed at 100 nM. B) Lyophilized mRBD-GlyIZ was subjected to transient thermal stress for 90 minutes followed by reconstitution in water. The lyophilized protein was reconstituted in MilliQ grade water prior to thermal melt and SPR binding studies. The binding to ACE2-hFc was performed at 100 nM. ACE2-hFc immobilized was 800RU. In solution, mRBD-GlyIZ loses activity upon exposure to temperatures higher than 40° C. The molecule also loses activity upon lyophilization and resolubilization, in accordance with an embodiment of the present disclosure.

FIG. 20 depicts ELISA and pseudovirus neutralization with sera elicited at weeks 0, 3 after two immunizations with SWE adjuvant containing formulations. A) and B) Immunization with mRBD (white panel) or hCMP-mRBD (SEQ ID NO: 14) (gray panel). C-E) Immunizations with mRBD-hCMP (SEQ ID NO: 16), mRBD-GlyIZ or MsDPS2 nanoparticle displaying mRBD. Pseudoviral neutralization titers utilized pNL4-3.Luc. SARS-CoV-2 D614G A19. HCS: Human Convalescent Sera (n=40). E) ELISA binding titer against scaffolds hCMP, GlyIZ trimerization domain, MsDPS2 SpyTag, and SpyCatcher. F-J) Pseudoviral neutralization titers against wildtype and pseudovirus with South African (B.1.351) RBD mutations. The paired comparisons were performed utilizing the Wilcoxon Rank-Sum test in F-G. The black solid horizontal lines in each scatter plot represent Geometric Mean Titer (GMT). The pairwise titer comparisons were performed utilizing two-tailed Mann-Whitney test in A-E (* indicates P<0.05, ** indicates P<0.01, **** indicates P<0.0001). K) Neutralizing antibody titers in mice (depicted in blue), in Human Convalescent Sera (HCS) (depicted in red) assayed in the identical assay platform, and their relative ratio (green). Values for a number of vaccine candidates being tested in the clinic or provided with emergency use authorizations are shown and corresponding values for hCMP-RBD are boxed, in accordance with an embodiment of the present disclosure.

FIG. 21 depicts hCMP-mRBD (SEQ ID NO: 14) adjuvant comparisons. Mice were immunized at week 0 and 3 with 20 μg of hCMP-mRBD adjuvanted with AddaVax™ and SWE. At 14 days post boost, sera were assayed for A) ELISA binding titer against mRBD. B) Pseudoviral neutralization titer utilizing pNL4-3.Luc. SARS-CoV-2 D614G Δ19, in accordance with an embodiment of the present disclosure.

FIG. 22 depicts the immunogenicity of CHO and Pichia expressed hCMP-RBD. A) SDS-PAGE of hCMP-pRBD purified from P. pastoris under reducing (+) and non-reducing (−) conditions. B) Mice were immunized at week 0, 3 with 20 μg of hCMP-mRBD (CHO) or hCMP-pRBD adjuvanted with the Addavax equivalent SWE adjuvant. At day 14 post boost, sera were assayed for ELISA binding titers to mRBD. C. Pseudoviral neutralization titer utilizing pNL4-3.Luc. SARS-CoV-2 D614G Δ19. The black horizontal lines in each scatter plot represent Geometric mean titer (GMT). The pairwise titer comparisons were performed utilizing two-tailed Mann-Whitney test (** indicates P<0.01), in accordance with an embodiment of the present disclosure.

FIG. 23 depicts guinea pig immunizations. Guinea pigs were immunized at week 0, 3 and 6 with 20 μg of trimeric hCMP-mRBD (SEQ ID NO: 14) adjuvanted with AddaVax™. A) 14 days post boost, sera were assayed for ELISA binding titer against mRBD. B) Pseudoviral neutralization titer utilizing pNL4-3.Luc. SARS-CoV-2 D614G Δ19. C) ELISA binding titer against scaffold hCMP. D-E) Pseudoviral neutralization titer utilizing the wildtype and South African (B.1.351) derived pseudovirus with sera obtained 14 days post second boost with D) hCMP-mRBD. E) Spike-2P. The pairwise titer comparisons were performed utilizing two-tailed Mann-Whitney test in A (** indicates P<0.01) and in D, E were performed utilizing paired two-tailed student-t test (* indicates P<0.05), in accordance with an embodiment of the present disclosure.

FIG. 24 depicts pseudoviral neutralization titer correlations, in accordance with an embodiment of the present disclosure.

FIG. 25 depicts hamster Immunization and challenge studies with trimeric hCMP-mRBD (SEQ ID NO: 14). Hamsters were immunized at week 0, 3 and 6 with 20 μg of hCMP-mRBD adjuvanted with AddaVax™. A) At 14 days post boost, sera were assayed for ELISA binding titer against mRBD and pseudoviral neutralization titer utilizing pNL4-3.Luc. SARS-CoV-2 D614G Δ19. B) ELISA binding titer against scaffold hCMP. Post immunization, the hamsters were challenged intranasally with replicative SARS-CoV-2 virus (10⁶ pfu/hamster) and monitored for C) Weight change D) Clinical signs E) Lung viral titer. Histopathology scores including F) Lung pathology score G) Inflammation score H) Immune cell influx score I) Edema score. The pairwise titer comparisons were performed utilizing two-tailed student-t test (* indicates P<0.05, ** indicates P<0.01), in accordance with an embodiment of the present disclosure.

FIG. 26 depicts SDS-PAGE of purified hCMP-mRBD (SEQ ID NO: 14) in reducing and non-reducing conditions. Protein was purified from transiently transfected Expi293F and stable cell lines Flp-in-293 and Flp-in-CHO. The black and red arrows represent the reduced and non-reduced protein bands respectively. The two red arrows likely indicate variably glycosylated forms, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably. The term “pharmaceutically acceptable carrier” refers to any known carrier, excipients, adjuvants known to a person skilled in the art, which can be used for preparing vaccines. The term “pharmaceutically effective amount” refers to an amount that is effective in eliciting the immune response using the vaccine as described in the present disclosure.

The term “SARS-CoV-2” refers to severe acute respiratory syndrome coronavirus 2. The term “COVID-19” refers to coronavirus diseases 2019.

The term “immunogenic composition” refers to a composition comprising the polypeptide fragment along with adjuvant and other excipients that elicits a prophylactic or therapeutic immune response in a subject. In the present disclosure, the “immunogenic composition” and “vaccine” are used interchangeably.

Typically, a vaccine elicits an antigen-specific immune response to an antigen of a pathogen, for example a viral pathogen, or to a cellular constituent correlated with a pathological condition.

The term “vaccine candidate” refers to a polypeptide fragment that can be potentially used in a vaccine composition.

The term “subject” refers to any animal classified as a mammal, e.g., human and non-human mammals. Examples of non-human animals include non-human primates, dogs, cats, cattle, horses, sheep, pigs, goats, rabbits, mice, rats, hamsters, guinea pigs and etc. Unless otherwise noted, the terms “patient” or “subject” are used herein interchangeably. Preferably, the subject is human.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

Current approaches for producing a vaccine against SARS-CoV-2 suffer from problems as described below.

Messenger RNA (mRNA) vaccines: In this approach, a formulation of the mRNA encoding the antigens of interest is used. The mRNA which is a highly charge molecule has to enter cells, be translated into protein and then be either exported outside the cell or processed inside the cell to stimulate humoral or cellular immunity respectively. Additionally, cost and scalability are uncertain.

DNA vaccines: In this approach, instead of mRNA, DNA is used for preparing vaccine formulation. Similar to the mRNA, the DNA has to enter the cell nucleus, undergo transcription and translation to yield the antigens of interest. While this approach works well in mice, immunogenicity in humans for DNA vaccines is typically not very high and there is a small but non-zero chance of genomic integration. There is also currently no DNA vaccine that has been approved for human use. A DNA vaccine encoding the SARS-CoV-2 spike protein has been tested in mice and guinea pigs PMID: 32433465 and shows good immunogenicity, however, results from human trials are awaited.

Viral vectors: In this approach, the gene(s) of interest are incorporated into a non-pathogenic virus capable of infecting cells. This may be either a replicating or non-replicating vector, typically the latter are preferred. Upon infection the genetic material is replicated, and any encoded protein antigens are expressed as with the mRNA and DNA vaccines discussed above. An advantage with this approach is that viral infection is very efficient, the disadvantage is that anti-vector immunity arises rapidly and so only a limited number of boosting immunizations are possible.

Live attenuated virus: In this approach, an attenuated (weakened) form of the virus is used. In the case of SARS-CoV-2, a process called codon-deoptimization is being used to generate such a weakened virus. This process takes time and extensive safety testing will be required for a highly pathogenic, novel virus such as in the present instance.

Inactivated virus: This is standard methodology for many vaccines. However, large amounts of pathogenic virus may need to be handled and some earlier studies with SARS-CoV have suggested the possibility of immune enhancement of infection when the inactivated virus was used as a vaccine modality.

There are currently multiple COVID-19 vaccines that have been given approval under emergency use and others with encouraging phase I data are in advanced clinical trials. It is pertinent to note that all COVID-19 vaccines in clinical use employ the full-length spike as the primary antigen. The sera from vaccines show a substantial decrease or even a complete loss of neutralization against the recent South African B.1.351 viral strain, primarily as a consequence of three mutations in the spike receptor binding domain (RBD). Therefore, despite these multiple efforts, there still remains a need for cheap, efficacious, COVID-19 vaccines that do not require a cold chain and elicit antibodies capable of neutralizing emerging variants of concern (VOC). Also, despite the extraordinarily rapid pace of vaccine development, there are currently many countries where not even a single dose has been administered. This will prolong the pandemic and promote viral evolution and escape. Thus, minimizing the extent of non-SARS-CoV-2 derived immunogenic sequence in the vaccine is highly desirable.

To circumvent the aforementioned problems, the present disclosure discloses an immunogenic composition used in form of a vaccine, wherein the immunogenic composition is developed under the category of subunit vaccines. This is a standard vaccine modality wherein purified protein(s) formulated with a suitable adjuvant comprise the vaccine. Protein yields need to be high enough and typically a suitable, human compatible adjuvant needs to be employed.

The present disclosure describes a recombinantly produced vaccine candidate (polypeptide) that is expressed in high yield in various host cells, including, but not limited to mammalian cells, insect cells, Pichia. Pastoris, and bacterial cells, and elicits high titer neutralizing antibodies against SARS-CoV-2 infection. The present disclosure discloses different polypeptide versions with addition or deletion of N-terminal glycosylation site leading to nCV01R (RBD1; 331-532) and nCV02R (RBD2; 332-532) versions, and third version with deletion of N and C-terminal glycosylation sites leading to nCV22R (RBD3; 332-530).

The polypeptide is a glycan engineered RBD derivative of SARS-CoV-2 comprising sequence from residues 332-532 of the spike protein is expressed using transient transfection in mammalian cells with a yield of ˜200 mg/liter, in insect cells with a yield of 60 mg/liter as well as in the yeast Pichia pastoris, with a purified yield of ˜25 mg/liter. The said polypeptide (glycan engineered RBD derivative) is highly thermotolerant and induced moderate to high titers of neutralizing antibodies. The protein binds hAce2 with a Kd of about 15 nM, is monomeric, is stable to lyophilization and redissolution, freeze thaw, 37° C. overnight incubation, and up to 1 hour incubation with trypsin at 37° C. Sera obtained from immunized animals with one of the RBD design formulations with a generic version of the human compatible MF59 vaccine adjuvant, was tested in viral neutralization assays, and showed neutralization titers of about 500.

Further, in order to improve the immunogenicity without negatively altering biophysical and antigenic characteristics of the designed immunogen, the present disclosure also discloses a thermotolerant intermolecular disulfide-linked, trimeric RBD derivatives. In an example of the present disclosure, there is provided a trimeric mRBD derivative (hCMP-mRBD; SEQ ID NO: 14), wherein the thermotolerant RBD is fused to a trimerization motif, namely a disulphide linked coiled-coil trimerization domain derived from human cartilage matrix protein (hCMP), to the N-terminus of mRBD. Alternatively, other trimerization domains, such as, chicken cartilage matrix protein (cCMP), or a fish cartilage matrix protein (FICMP), or a fish isoform 2 cartilage matrix protein (F2-CMP), foldon, Leucine Zipper with double cysteine (CCIZ), Synthetic trimerization domain (cCMP-IZₘ), Glycosylated leucine zipper sequence (Gly IZ) can also be fused at either N or C-terminal of RBD residues (RBD1 (331-532), or RBD2 (332-532), or RBD3 (332-530)). The trimeric RBD derivatives, such as, hCMP-mRBD expressed as homogenous trimers in mammalian cells, insect cells, and the Pichia pastoris, possessed comparable thermal stability profiles to the corresponding monomer and remained functional after over 4 weeks upon lyophilization and storage at 37° C. The trimeric RBD is highly immunogenic in mice and guinea pigs when formulated with SWE adjuvant. SWE is equivalent to the widely used, clinically approved, MF59 adjuvant. Oligomerization increased neutralizing antibody titers by approximately 25-250 folds when compared with the titers in human convalescent sera, providing a proof of principle for the design strategy. Further the hCMP-mRBD protected hamsters from viral challenge, and immunized sera from mice and guinea pigs neutralized the rapidly spreading South African (B.1.351) viral variant with only a three-fold decrease in neutralization titers. Stable CHO and HEK293 cell lines expressing hCMP-mRBD were constructed and the corresponding protein was as immunogenic, as the protein expressed from transient transfection. The very high thermotolerance, enhanced immunogenicity, and protection from viral challenge suggest that trimeric RBD derivatives such as (hCMP-mRBD) with inter-subunit, stable disulfides, is an attractive vaccine candidate that can be deployed to combat COVID-19 without requirement of a cold-chain, especially in resource limited settings.

The present disclosure also discloses various variants of polypeptides having one or more mutations. The mutations are identified in polypeptide having amino acid sequence selected from the group consisting of SEQ ID NO: 2 (331-532; RBD1), SEQ ID NO: 4 (332-532; RBD2), SEQ ID NO: 6 (332-530; RBD3). Further, the mutations are also identified in the polypeptide having amino acid sequence selected from the group consisting of SEQ ID NO: 8 (mInCV01R; variant of SEQ ID NO: 2), SEQ ID NO: 10 (mInCV02R; variant of SEQ ID NO: 4), SEQ ID NO: 12 (mInCV22R; variant of SEQ ID NO: 6). The polypeptide (vaccine candidate) having one or more mutations is expressed in high yield in mammalian cells, insect cells, and the Pichia. Pastoris.

Table 1 shows the amino acid abbreviations.

TABLE 1

| Amino acid | Three letter code | One letter code |
|---|---|---|
| Alanine | ala | A |
| Arginine | arg | R |
| Asparagine | asn | N |
| Aspartic acid | asp | D |
| Cysteine | cys | C |
| Glutamic acid | glu | E |
| Glutamine | gln | Q |
| Glycine | gly | G |
| Histidine | his | H |
| Isoleucine | ile | I |
| Leucine | leu | L |
| Lysine | lys | K |
| Methionine | met | M |
| Phenylalanine | phe | F |
| Proline | pro | P |
| Serine | ser | S |
| Threonine | thr | T |
| Tryptophan | trp | W |
| Tyrosine | tyr | Y |
| Valine | val | V |

Mutations or variations are described by use of the following nomenclature: position: amino acid residue in the protein scaffold; position; substituted amino acid residue(s). According to this nomenclature, the substitution of, for the substitution of, for instance, a threonine residue for a histidine residue at position 333 of RBD residue is indicated as Thr333His or T333H, or 333H. Similarly, it can also be appreciated that when there is a substitution of a threonine residue for a histidine residue in polypeptide having an amino acid sequence as set forth in SEQ ID NO: 2 (331-532), then such mutation is indicated with a nomenclature of Thr3His or T3H, or 3H.

When an amino acid residue at a given position is substituted with two or more alternative amino acid residues, then these residues are separated by a comma or a slash. For example, two mutations in positions 527 and 365 substituting proline and tyrosine with leucine and phenylalanine, respectively are indicated as P527L/Y365F.

Such mutations help in improving the manufacturability of RBD-based immunogenic composition (vaccines) and also helps in improving the expression of protein in host cells and also enhancing the thermal stability. Such modification in the polypeptide is crucial for maximizing the scale and speed of vaccine production and buffering against the anticipated changes in the stability and solution properties of antigens derived from SARS-CoV-2 isolates.

In an embodiment of the present disclosure, there is provided a polypeptide fragment having an amino acid sequence with at least 95% identity to the amino acid sequence selected from the group consisting of SEQ ID NO: 2, SEQ ID NO: 4, and SEQ ID NO: 6. In another embodiment of the present disclosure, the identity is 96%, 97%, 98%, 99%, 99.5% to the amino acid sequence selected from the group consisting of SEQ ID NO: 2, SEQ ID NO: 4, and SEQ ID NO: 6.

In an embodiment of the present disclosure, there is provided a polypeptide fragment having an amino acid sequence with at least 95% identity to the amino acid sequence as set forth in SEQ ID NO: 2. In another embodiment of the present disclosure, the identity is 96%, 97%, 98%, 99%, 99.5% to the amino acid sequence selected from the group consisting of SEQ ID NO: 2.

In an embodiment of the present disclosure, there is provided a polypeptide fragment having an amino acid sequence with at least 95% identity to the amino acid sequence as set forth in SEQ ID NO: 4. In another embodiment of the present disclosure, the identity is 96%, 97%, 98%, 99%, 99.5% to the amino acid sequence as set forth in SEQ ID NO: 4.

In an embodiment of the present disclosure, there is provided a polypeptide fragment having an amino acid sequence with at least 95% identity to the amino acid sequence as set forth in SEQ ID NO: 6. In another embodiment of the present disclosure, the identity is 96%, 97%, 98%, 99%, 99.5% to the amino acid sequence as set forth in SEQ ID NO: 6.

In an embodiment of the present disclosure, there is provided a polypeptide fragment comprising a polypeptide having an amino acid sequence selected from the group consisting of SEQ ID NO: 2, SEQ ID NO: 4, or SEQ ID NO: 6.

In an embodiment of the present disclosure, there is provided a polypeptide fragment comprising a polypeptide having an amino acid sequence selected from the group consisting of SEQ ID NO: 8, SEQ ID NO: 10, or SEQ ID NO: 12.

In an embodiment of the present disclosure, there is provided a polypeptide fragment comprising a polypeptide having an amino acid sequence selected from the group consisting of SEQ ID NO: 2; or SEQ ID NO: 8.

In an embodiment of the present disclosure, there is provided a polypeptide fragment comprising a polypeptide having an amino acid sequence as set forth in SEQ ID NO: 8.

In an embodiment of the present disclosure, there is provided a polypeptide fragment comprising a polypeptide having a substitution at an amino acid position in SEQ ID NO: 2, wherein the substitution at the amino acid position is selected from the group consisting of positions at 3, 7, 16, 18, 24, 28, 35, 37, 39, 42, 43, 53, 55, 59, 60, 62, 78, 84, 98, 100, 104, 129, 130, 134, 138, 147, 190, and 197.

In an embodiment of the present disclosure, there is provided a polypeptide fragment comprising a polypeptide having a substitution at an amino acid position in SEQ ID NO: 8, wherein the substitution at the amino acid position is selected from the group consisting of positions at 6, 10, 19, 21, 27, 31, 38, 40, 42, 45, 46, 56, 58, 62, 63, 65, 81, 87, 101, 103, 107, 132, 133, 137, 141, 150, 193, and 200;

In an embodiment of the present disclosure, there is there is provided a polypeptide fragment comprising a polypeptide having a substitution at an amino acid position in SEQ ID NO: 2, wherein the substitution at the amino acid position is selected from the group consisting of positions at 3, 7, 16, 18, 24, 28, 35, 37, 39, 42, 43, 53, 55, 59, 60, 62, 78, 84, 98, 100, 104, 129, 130, 134, 138, 147, 190, and 197 corresponding to T3H, P7D, R16T, A18P, N24E, I28F, Y35F, V37F, Y39L, A42M, S43K, S53D, T55S, D59E, L60M, F62W, R78D, I84F, D98N, T100V, Q104A, S129Q, N130V, F134Y, I138V, S147E, A190G, and P197L, respectively. In another embodiment of the present disclosure, the substitution at the amino acid position is selected from the group consisting of positions at 16, 35, 42, 55, 138, and 197 corresponding to R16K, Y35W, A42T, T55E, I138T, P197T, respectively. In yet another embodiment of the present disclosure, the substitution at the amino acid position at 197 corresponds to P197I.

In an embodiment of the present disclosure, there is there is provided a polypeptide fragment comprising a polypeptide having a substitution at an amino acid position in SEQ ID NO: 8, wherein the substitution at the amino acid position is selected from the group consisting of positions at 6, 10, 19, 21, 27, 31, 38, 40, 42, 45, 46, 56, 58, 62, 63, 65, 81, 87, 101, 103, 107, 132, 133, 137, 141, 150, 193, and 200 corresponding to T6H, P10D, R19T, A21P, N27E, I31F, Y38F, V40F, Y42L, A45M, S46K, S56D, T58S, D62E, L63M, F65W, R81D, I87F, D101N, T103V, Q107A, S132Q, N133V, F137Y, I141V, S150E, A193G, and P200L, respectively. In another embodiment of the present disclosure, the substitution at the amino acid position is selected from the group consisting of positions at 19, 38, 45, 58, 141, and 200 corresponding to R19K, Y38W, A45T, T58E, I141T, P200T, respectively. In yet another embodiment of the present disclosure, the substitution at the amino acid position at 200 corresponds to P200I.

In an embodiment of the present disclosure, there is there is provided a polypeptide fragment comprising a polypeptide having at least one variation in the amino acid sequence as set forth in SEQ ID NO: 2, wherein the at least one variation is selected from the group consisting of P197R/K198R/K199V/S200P/N202V, P197L/Y35F, P197L/A190G/Y35F, P197L/A190G/Y35F/T3H, P197L/A190G/Y35F/T3H/T55S, P197L/A190G/Y35F, P197L/A190G/Y35F/T3H/T55S/V173D, A18P/P197L/A190G/Y35F/T3H, A18P/A42M/P197L/A190G/Y35F/T3H, A18P/A42M/T100V/P197L/A190G/Y35F/T3H, Y35W/L60M/N118D/Q163S/C195D, A18P/Y35W/P197L, A18P/V37F/P197L, A18P/Y35W/V37F/P197L, A18P/V37F/P197I, A18P/Y35W/V37F/P197I, N13D/A18P/V37F/P197L, N13D/A18P/Y35W/P197L, I28F/Y35W, I28F/F62W, I28F/I104F, Y35W/Y62W, Y35W/I104F, Y62W/I104F, I28F/Y35W/F62W, I28F/Y35W/I104F, I28F/F62W/I104F, Y35W/F62W/I104F, or I28F/Y35W/F62W/I104F.

In an embodiment of the present disclosure, there is there is provided a polypeptide fragment comprising a polypeptide having at least one variation in the amino acid sequence as set forth in SEQ ID NO: 8, wherein the at least one variation is selected from the group consisting of P200R/K201R/K202V/S203P/N205V, P200L/Y38F, P200L/A193G/Y38F, P200L/A193G/Y38F/T6H, P200L/A193G/Y38F/T6H/T 58S, P200L/A193G/Y38F/T6H/T58S/V176D, A21P/P200L/A193G/Y38F/T6H, A21P/A45M/P200L/A193G/Y38F/T6H, A21P/A45M/T103V/P200L/A193G/Y38F/T6H, Y38W/L63M/N121D/Q166S/C198D, A21P/V40F/P200L, A21P/Y38W/V40F/P200L, A21P/Y38W/P200L, A21P/V40F/P200I, A21P/Y38W/V40F/P200I, N16D/A21P/V40F/P200L, N16D/A21P/Y38W/P200L,

I31F/Y37W, I31F/F65W, I31F/I107F, Y38W/Y65W, Y38W/I107F, Y65W/I107F, I31F/Y38W/F65W, I31F/Y38W/I107F, I31F/F65W/I107F, Y38W/F65W/I107F, or I31F/Y38W/F65W/I107F.

In an embodiment of the present disclosure, there is there is provided a polypeptide fragment comprising a polypeptide having an amino acid selected from the group having the amino acid sequence as set forth in SEQ ID NO: 76, and SEQ ID NO: 79.

In an embodiment of the present disclosure, there is there is provided a polypeptide fragment comprising a polypeptide having an amino acid sequence selected from the group consisting of SEQ ID NO: 4, or SEQ ID NO: 10.

In an embodiment of the present disclosure, there is there is provided a polypeptide fragment comprising a polypeptide having an amino acid sequence as set forth in SEQ ID NO: 10.

In an embodiment of the present disclosure, there is there is provided a polypeptide fragment comprising a polypeptide having a substitution at an amino acid position in SEQ ID NO: 4, wherein the substitution at the amino acid position is selected from the group consisting of positions at 2, 6, 15, 17, 23, 27, 34, 36, 38, 41, 42, 52, 54, 58, 59, 61, 77, 97, 99, 128, 129, 133, 137, 146, 189, and 196.

In an embodiment of the present disclosure, there is there is provided a polypeptide fragment comprising a polypeptide having a substitution at an amino acid position in SEQ ID NO: 10, wherein the substitution at the amino acid position is selected from the group consisting of positions at 5, 9, 18, 20, 26, 30, 37, 39, 41, 44, 45, 55, 57, 61, 62, 64, 80, 100, 102, 131, 132, 136, 140, 149, 192, and 199.

In an embodiment of the present disclosure, there is there is provided a polypeptide fragment comprising a polypeptide having a substitution at an amino acid position in SEQ ID NO: 4, wherein the substitution at the amino acid position is selected from the group consisting of positions at 2, 6, 15, 17, 23, 27, 34, 36, 38, 41, 42, 52, 54, 58, 59, 61, 77, 83, 97, 99, 103, 128, 129, 133, 137, 146, 189, and 196 corresponding to T2H, P6D, R15T, A17P, N23E, I27F, Y34F, V36F, Y38L, A41M, S42K, S52D, T54S, D58E, L59M, F61W, R77D, I83F, D97N, T99V, Q103A, S128Q, N129V, F133Y, I137V, S146E, A189G, and P196L, respectively. In another embodiment of the present disclosure, the substitution at the amino acid position is selected from the group consisting of positions at 15, 34, 41, 54, 137, and 196 corresponding to R15K, Y34W, A41T, T54E, I137T, P196T, respectively. In yet another embodiment of the present disclosure, the substitution at the amino acid position at 196 corresponds to P196I.

In an embodiment of the present disclosure, there is there is provided a polypeptide fragment comprising a polypeptide having a substitution at an amino acid position in SEQ ID NO: 10, wherein the substitution at the amino acid position is selected from the group consisting of positions at 5, 9, 18, 20, 26, 30, 37, 39, 41, 44, 45, 55, 57, 61, 62, 64, 80, 86, 100, 102, 106, 131, 132, 136, 140, 149, 192, and 199 corresponding to TSH, P9D, R18T, A20P, N26E, I30F, Y37F, V39F, Y41L, A44M, S45K, S55D, T57S, D61E, L62M, F64W, R80D, I86F, D100N, T102V, Q106A, S131Q, N132V, F136Y, I140V, S149E, A192G, and P199L, respectively. In another embodiment of the present disclosure, the substitution at the amino acid position is selected from the group consisting of positions at 18, 37, 44, 57, 140, and 199 corresponding to R18K, Y37W, A44T, T57E, I140T, P199T, respectively. In yet another embodiment of the present disclosure, the substitution at the amino acid position at 199 corresponds to P199I.

In an embodiment of the present disclosure, there is there is provided a polypeptide fragment comprising a polypeptide having at least one variation in the amino acid sequence as set forth in SEQ ID NO: 4, wherein the at least one variation is selected from the group consisting of P196R/K197R/K198V/S199P/N201V, P196L/Y34F, P196L/A189G/Y34F, P196L/A189G/Y34F/T2H, P196L/A189G/Y34F/T2H/T54S, P196L/A189G/Y34F/T2H/T54S/V172D, A17P/P196L/A189G/Y34F/T2H, A17P/A41M/P196L/A189G/Y34F/T2H, A17P/A41M/T99V/P196L/A189G/Y34F/T2H, Y34W/L59M/N117D/Q162S/C194D, A17P/V36F/P196L, A17P/Y34W/V36F/P196L, A17P/Y34W/P196L, A17P/V36F/P196I, A17P/Y34W/V36F/P196I, N12D/A17P/V36F/P196L, N12D/A17P/Y34W/P196L, I27F/Y34W, I27F/F61W, I27F/I103F, Y34W/Y61W, Y34W/I103F, Y61W/I103F, I27F/Y34W/F61W, I27F/Y34W/I103F, I27F/F61W/I102F, Y34W/F61W/I103F, I27F/Y34W/F62W/I103F.

In an embodiment of the present disclosure, there is there is provided a polypeptide fragment comprising a polypeptide having at least one variation in the amino acid sequence as set forth in SEQ ID NO: 10, wherein the at least one variation is selected from the group consisting of P199R/K200R/K201V/S202P/N204V, P199L/Y37F, P199L/A192G/Y37F, P199L/A192G/Y37F/T5H, P199L/A192G/Y 37F/T5H/T57S, P199L/A192G/Y37F/T5H/T57S/V175D, A20P/P199L/A192G/Y37F/T5H, A20P/A44M/P199L/A192G/Y37F/T5H, A20P/A44M/T102V/P199L/A192G/Y37F/T5H, Y37W/L62M/N120D/Q165S/C197D, A20P/Y37W/P199L, A20P/V39F/P199L, A20P/Y37W/V39F/P199L, A20P/V39F/P199I, A20P/Y37W/V39F/P199I, N15D/A20P/V39F/P199L, N15D/A20P/Y37W/P199L, I30F/Y36W, I30F/F64W, I30F/I106F, Y37W/Y64W, Y37W/I106F, Y64W/I106F, I30F/Y37W/F64W, I30F/Y37W/I106F, I30F/F65W/I106F, Y37W/F64W/I106F, I30F/Y37W/F64W/I106F.

In an embodiment of the present disclosure, there is there is provided a polypeptide fragment comprising a polypeptide having an amino acid as set forth in SEQ ID NO: 77.

In an embodiment of the present disclosure, there is there is provided a polypeptide fragment comprising a polypeptide having an amino acid sequence selected from the group consisting of SEQ ID NO: 6, or SEQ ID NO: 12.

In an embodiment of the present disclosure, there is there is provided a polypeptide fragment comprising a polypeptide having an amino acid sequence as set forth in SEQ ID NO: 12.

In an embodiment of the present disclosure, there is there is provided a polypeptide fragment comprising a polypeptide having a substitution at an amino position in SEQ ID NO: 6, wherein the substitution at the amino acid position is selected from the group consisting of positions at 2, 6, 15, 17, 23, 27, 34, 36, 38, 41, 42, 52, 54, 58, 59, 61, 77, 97, 99, 128, 129, 133, 137, 146, 189, and 196.

In an embodiment of the present disclosure, there is there is provided a polypeptide fragment comprising a polypeptide having a substitution at an amino acid position in SEQ ID NO: 12, wherein the substitution at the amino acid position is selected from the group consisting of positions at 5, 9, 18, 20, 26, 30, 37, 39, 41, 44, 45, 55, 57, 61, 62, 64, 80, 100, 102, 110, 131, 132, 136, 140, 149, 192, and 199.

In an embodiment of the present disclosure, there is there is provided a polypeptide fragment comprising a polypeptide having a substitution at an amino position in SEQ ID NO: 6, wherein the substitution at the amino acid position is selected from the group consisting of positions at 2, 6, 15, 17, 23, 27, 34, 36, 38, 41, 42, 52, 54, 58, 59, 61, 77, 83, 97, 99, 128, 103, 129, 133, 137, 146, 189, and 196 to T2H, P6D, R15T, A17P, N23E, I27F, Y34F, V36F, Y38L, A41M, S42K, S52D, T54S, D58E, L59M, F61W, R77D, I83F, D97N, T99V, S128Q, Q103A, N129V, F133Y, I137V, S146E, A189G, and P196L, respectively. In another embodiment of the present disclosure, the substitution at the amino acid position is selected from the group consisting of positions at 15, 34, 41, 54, 137, and 196 corresponding to R15K, Y34W, A41T, T54E, I137T, P196T, respectively. In yet another embodiment of the present disclosure, the substitution at the amino acid position at 196 corresponds to P196I.

In an embodiment of the present disclosure, there is there is provided a polypeptide fragment comprising a polypeptide having a substitution at an amino acid position in SEQ ID NO: 12, wherein the substitution at the amino acid position is selected from the group consisting of positions at 5, 9, 18, 20, 26, 30, 37, 39, 41, 44, 45, 55, 57, 61, 62, 64, 80, 86, 100, 102, 106, 110, 131, 132, 136, 140, 149, 192, and 199 corresponding to T5H, P9D, R18T, A20P, N26E, I30F, Y37F, V39F, Y41L, A44M, S45K, S55D, T57S, D61E, L62M, F64W, R80D, I86F, D100N, T102V, Q106A, S131Q, N132V, F136Y, I140V, S149E, A192G, and P199L, respectively. In another embodiment of the present disclosure, the substitution at the amino acid position is selected from the group consisting of positions at 18, 37, 44, 57, 140, and 199 corresponding to R18K, Y37W, A44T, T57E, I140T, P199T, respectively. In yet another embodiment of the present disclosure, the substitution at the amino acid position at 199 corresponds to P199I.

In an embodiment of the present disclosure, there is there is provided a polypeptide fragment comprising a polypeptide having at least one variation in the amino acid sequence as set forth in SEQ ID NO: 6, wherein the at least one variation is selected from the group consisting of P196R/K197R/K198V/S199P/N201V, P196L/Y34F, P196L/A189G/Y34F, P196L/A189G/Y34F/T2H, P196L/A189G/Y34F/T2H/T54S, P196L/A189G/Y34F/T2H/T54S/V172D, A17P/P196L/A189G/Y34F/T2H, A17P/A41M/P196L/A189G/Y34F/T2H, A17P/A41M/T99V/P196L/A189G/Y34F/T2H, Y34W/L59M/N117D/Q162S/C194D, A17P/Y34W/P196L, A17P/V36F/P196L, A17P/Y34W/V36F/P196L, A17P/V36F/P196I, A17P/Y34W/V36F/P196I, N12D/A17P/V36F/P196L, N12D/A17P/Y34W/P196L, I27F/Y34W, I27F/F61W, I27F/I103F, Y34W/Y61W, Y34W/I103F, Y61W/I103F, I27F/Y34W/F61W, I27F/Y34W/I103F, I27F/F61W/I102F, Y34W/F61W/I103F, and I27F/Y34W/F62W/I103F.

In an embodiment of the present disclosure, there is there is provided a polypeptide fragment comprising a polypeptide having at least one variation in the amino acid sequence as set forth in SEQ ID NO: 12, wherein the at least one variation is selected from the group consisting of P199R/K200R/K201V/S202P/N204V, P199L/Y37F, P199L/A192G/Y37F, P199L/A192G/Y37F/T5H, P199L/A192G/Y 37F/T5H/T57S, P199L/A192G/Y37F/T5H/T57S/V175D, A20P/P199L/A192G/Y37F/T5H, A20P/A44M/P199L/A192G/Y37F/T5H, A20P/A44M/T102V/P199L/A192G/Y37F/T5H, Y37W/L62M/N120D/Q165S/C197D, A20P/Y37W/P199L, A20P/V39F/P199L, A20P/Y37W/V39F/P199L, A20P/V39F/P199I, A20P/Y37W/V39F/P199I, N15D/A20P/V39F/P199L, N15D/A20P/Y37W/P199L, I30F/Y36W, I30F/F64W, I30F/I106F, Y37W/Y64W, Y37W/I106F, Y64W/I106F, I30F/Y37W/F64W, I30F/Y37W/I106F, I30F/F65W/I106F, Y37W/F64W/I106F, and I30F/Y37W/F64W/I106F.

In an embodiment of the present disclosure, there is there is provided a polypeptide fragment comprising a polypeptide having an amino acid selected from the group having the amino acid sequence as set forth in SEQ ID NO: 69, SEQ ID NO: 70, SEQ ID NO: 71, SEQ ID NO: 72, SEQ ID NO: 73, SEQ ID NO: 74, and SEQ ID NO: 85.

In an embodiment of the present disclosure, there is there is provided a polypeptide fragment comprising a polypeptide having an amino acid sequence selected from the group consisting of SEQ ID NO:14, SEQ ID NO: 16, SEQ ID NO: 18, SEQ ID NO: 20, and SEQ ID NO: 22.

In an embodiment of the present disclosure, there is there is provided a polypeptide fragment comprising a polypeptide having an amino acid sequence selected from the group consisting of SEQ ID NO: 81, and SEQ ID NO: 83.

In an embodiment of the present disclosure, there is there is provided a polypeptide fragment comprising a polypeptide having an amino acid sequence selected from the group consisting of SEQ ID NO: 56, SEQ ID NO: 58, and SEQ ID NO: 60.

In an embodiment of the present disclosure, there is there is provided a polypeptide fragment comprising a polypeptide having an amino acid sequence selected from the group consisting of SEQ ID NO: 52, SEQ ID NO: 54, SEQ ID NO: 62, SEQ ID NO: 64, SEQ ID NO: 66, and SEQ ID NO: 68.

In an embodiment of the present disclosure, there is there is provided a polypeptide fragment comprising a polypeptide having an amino acid sequence selected from the group consisting of SEQ ID NO: 24, SEQ ID NO: 26, SEQ ID NO: 28, SEQ ID NO: 30, SEQ ID NO: 32, SEQ ID NO: 34, SEQ ID NO: 38, SEQ ID NO: 40, SEQ ID NO: 42, SEQ ID NO: 44, SEQ ID NO: 46, SEQ ID NO: 48, and SEQ ID NO: 50.

In an embodiment of the present disclosure, there is there is provided a polypeptide fragment comprising a polypeptide fragment comprising a polypeptide having an amino acid sequence selected from the group consisting of SEQ ID NO: 8, SEQ ID NO: 10, SEQ ID NO: 12, SEQ ID NO: 14, SEQ ID NO: 69, SEQ ID NO: 70, SEQ ID NO: 71, SEQ ID NO: 73, SEQ ID NO: 74, SEQ ID NO: 76, SEQ ID NO: 77, SEQ ID NO: 79, SEQ ID NO: 81, SEQ ID NO: 83, and SEQ ID NO: 85.

In an embodiment of the present disclosure, there is provided a recombinant construct comprising the nucleic acid fragment encoding a polypeptide fragment as described herein, operably linked to a promoter.

In an embodiment of the present disclosure, there is provided a recombinant construct comprising the nucleic acid fragment, said nucleic acid fragment encoding a polypeptide fragment, said polypeptide fragment having an amino acid sequence with at least 95% identity to the amino acid sequence selected from the group consisting of SEQ ID NO: 2, SEQ ID NO: 4, and SEQ ID NO: 6, operably linked to a promoter.

In an embodiment of the present disclosure, there is provided a recombinant construct comprising the nucleic acid fragment, said nucleic acid fragment encoding a polypeptide fragment, said polypeptide fragment having an amino acid sequence selected from the group consisting of SEQ ID NO: 2, SEQ ID NO: 4, SEQ ID NO: 6, SEQ ID NO: 8, SEQ ID NO: 10, or SEQ ID NO: 12, operably linked to a promoter.

In an embodiment of the present disclosure, there is provided a recombinant construct comprising the nucleic acid fragment, said nucleic acid fragment encoding a polypeptide fragment, said polypeptide fragment comprises: (a) a polypeptide having an amino acid sequence selected from the group consisting of SEQ ID NO: 2; or SEQ ID NO: 8; (b) a polypeptide having a substitution at an amino acid position in SEQ ID NO: 2, wherein the substitution at the amino acid position is selected from the group consisting of positions at 3, 7, 16, 18, 24, 28, 35, 37, 39, 42, 43, 53, 55, 59, 60, 62, 78, 98, 100, 129, 130, 134, 138, 147, 190, and 197; (c) a polypeptide having a substitution at an amino acid position in SEQ ID NO: 8, wherein the substitution at the amino acid position is selected from the group consisting of positions at 6, 10, 19, 21, 27, 31, 38, 40, 42, 45, 46, 56, 58, 62, 63, 65, 81, 101, 103, 132, 133, 137, 141, 150, 193, and 200; (d) a polypeptide having at least one variation in the amino acid sequence as set forth in SEQ ID NO: 2, wherein the at least one variation is selected from the group consisting of P197R/K198R/K199V/S200P/N202V, P197L/Y35F, P197L/A190G/Y35F, P197L/A190G/Y35F/T3H, P197L/A190G/Y35F/T 3H/T55S, P197L/A190G/Y35F, P197L/A190G/Y35F/T3H/T55S/V173D, A18P/P197L/A190G/Y35F/T3H, A18P/A42M/P197L/A190G/Y35F/T3H, A18P/A42M/T100V/P197L/A190G/Y35F/T3H, Y35W/L60M/N118D/Q163S/C195D, A18P/V37F/P197L, A18P/Y35W/V37F/P197L, A18P/V37F/P197I, A18P/Y35W/V37F/P197I, N13D/A18P/V37F/P197L, N13D/A18P/Y35W/P197L, I28F/Y35W, I28F/F62W, I28F/I104F, Y35W/Y62W, Y35W/I104F, Y62W/I104F, I28F/Y35W/F62W, I28F/Y35W/I104F, I28F/F62W/I104F, Y35W/F62W/I104F, or I28F/Y35W/F62W/I104F; (e) a polypeptide having at least one variation in the amino acid sequence as set forth in SEQ ID NO: 8, wherein the at least one variation is selected from the group consisting of P200R/K201R/K202V/S203P/N205V, P200L/Y38F, P200L/A193G/Y38F, P200L/A193G/Y38F/T6H, P200L/A193G/Y38F/T6H/T 58S, P200L/A193G/Y38F/T6H/T58S/V176D, A21P/P200L/A193G/Y38F/T6H, A21P/A45M/P200L/A193G/Y38F/T6H, A21P/A45M/T103V/P200L/A193G/Y38F/T6H, Y38W/L63M/N121D/Q166S/C198D, A21P/V40F/P200L, A21P/Y38W/V40F/P200L, A21P/V40F/P200I, A21P/Y38W/V40F/P200I, N16D/A21P/V40F/P200L, N16D/A21P/Y38W/P200L, I31F/Y37W, I31F/F65W, I31F/I107F, Y38W/Y65W, Y38W/I107F, Y65W/I107F, I31F/Y38W/F65W, I31F/Y38W/I107F, I31F/F65W/I107F, Y38W/F65W/I107F, or I31F/Y38W/F65W/I107F; or (f) a polypeptide having an amino acid selected from the group having the amino acid sequence as set forth in SEQ ID NO: 76, and SEQ ID NO: 79, operably linked to a promoter.

In an embodiment of the present disclosure, there is provided a recombinant construct comprising the nucleic acid fragment, said nucleic acid fragment encoding a polypeptide fragment, said polypeptide fragment comprises: (a) a polypeptide having an amino acid sequence selected from the group consisting of SEQ ID NO: 4, or SEQ ID NO: 10; (b) a polypeptide having a substitution at an amino acid position in SEQ ID NO: 4, wherein the substitution at the amino acid position is selected from the group consisting of positions at 2, 6, 15, 17, 23, 27, 34, 36, 38, 41, 42, 52, 54, 58, 59, 61, 77, 97, 99, 128, 129, 133, 137, 146, 189, and 196; (c) a polypeptide having a substitution at an amino acid position in SEQ ID NO: 10, wherein the substitution at the amino acid position is selected from the group consisting of positions at 5, 9, 18, 20, 26, 30, 37, 39, 41, 44, 45, 55, 57, 61, 62, 64, 80, 100, 102, 131, 132, 136, 140, 149, 192, and 199; (d) a polypeptide having at least one variation in the amino acid sequence as set forth in SEQ ID NO: 4, wherein the at least one variation is selected from the group consisting of P196R/K197R/K198V/S199P/N201V, P196L/Y34F, P196L/A189G/Y34F, P196L/A189G/Y34F/T2H, P196L/A189G/Y34F/T2H/T54S, P196L/A189G/Y34F/T2H/T54S/V172D, A17P/P196L/A189G/Y34F/T2H, A17P/A41M/P196L/A189G/Y34F/T2H, A17P/A41M/T99V/P196L/A189G/Y34F/T2H, Y34W/L59M/N117D/Q162S/C194D, A17P/V36F/P196L, A17P/Y34W/V36F/P196L, A17P/V36F/P196I, A17P/Y34W/V36F/P196I, N12D/A17P/V36F/P196L, N12D/A17P/Y34W/P196L, I27F/Y34W, I27F/F61W, I27F/I103F, Y34W/Y61W, Y34W/I103F, Y61W/I103F, I27F/Y34W/F61W, I27F/Y34W/I103F, I27F/F61W/I102F, Y34W/F61W/I103F, I27F/Y34W/F62W/I103F; (e) a polypeptide having at least one variation in the amino acid sequence as set forth in SEQ ID NO: 10, wherein the at least one variation is selected from the group consisting of P199R/K200R/K201V/S202P/N204V, P199L/Y37F, P199L/A192G/Y37F, P199L/A192G/Y37F/T5H, P199L/A192G/Y 37F/T5H/T57S, P199L/A192G/Y37F/T5H/T57S/V175D, A20P/P199L/A192G/Y37F/T5H, A20P/A44M/P199L/A192G/Y37F/T5H, A20P/A44M/T102V/P199L/A192G/Y37F/T5H, Y37W/L62M/N120D/Q165S/C197D, A20P/V39F/P199L, A20P/Y37W/V39F/P199L, A20P/V39F/P199I, A20P/Y37W/V39F/P199I, N15D/A20P/V39F/P199L, N15D/A20P/Y37W/P199L, I30F/Y36W, I30F/F64W, I30F/I106F, Y37W/Y64W, Y37W/I106F, Y64W/I106F, I30F/Y37W/F64W, I30F/Y37W/I106F, I30F/F65W/I106F, Y37W/F64W/I106F, I30F/Y37W/F64W/I106F; or (f) a polypeptide having an amino acid as set forth in SEQ ID NO: 77, operably linked to a promoter.

In an embodiment of the present disclosure, there is provided a recombinant construct comprising the nucleic acid fragment, said nucleic acid fragment encoding a polypeptide fragment, said polypeptide fragment comprises: (a) a polypeptide having an amino acid sequence selected from the group consisting of SEQ ID NO: 6, or SEQ ID NO: 12; (b) a polypeptide having a substitution at an amino acid position in SEQ ID NO: 6, wherein the substitution at the amino acid position is selected from the group consisting of positions at 2, 6, 15, 17, 23, 27, 34, 36, 38, 41, 42, 52, 54, 58, 59, 61, 77, 97, 99, 128, 129, 133, 137, 146, 189, and 196; (c) a polypeptide having a substitution at an amino acid position in SEQ ID NO: 12, wherein the substitution at the amino acid position is selected from the group consisting of positions at 5, 9, 18, 20, 26, 30, 37, 39, 41, 44, 45, 55, 57, 61, 62, 64, 80, 100, 102, 110, 131, 132, 136, 140, 149, 192, and 199; (d) a polypeptide having at least one variation in the amino acid sequence as set forth in SEQ ID NO: 6, wherein the at least one variation is selected from the group consisting of P196R/K197R/K198V/S199P/N201V, P196L/Y34F, P196L/A189G/Y34F, P196L/A189G/Y34F/T2H, P196L/A189G/Y34F/T2H/T54S, P196L/A189G/Y34F/T2H/T54S/V172D, A17P/P196L/A189G/Y34F/T2H, A17P/A41M/P196L/A189G/Y34F/T2H, A17P/A41M/T99V/P196L/A189G/Y34F/T2H, Y34W/L59M/N117D/Q162S/C194D, A17P/V36F/P196L, A17P/Y34W/V36F/P196L, A17P/V36F/P196I, A17P/Y34W/V36F/P196I, N12D/A17P/V36F/P196L, N12D/A17P/Y34W/P196L, I27F/Y34W, I27F/F61W, I27F/I103F, Y34W/Y61W, Y34W/I103F, Y61W/I103F, I27F/Y34W/F61W, I27F/Y34W/I103F, I27F/F61W/I102F, Y34W/F61W/I103F, and I27F/Y34W/F62W/I103F; (e) a polypeptide having at least one variation in the amino acid sequence as set forth in SEQ ID NO: 12, wherein the at least one variation is selected from the group consisting of P199R/K200R/K201V/S202P/N204V, P199L/Y37F, P199L/A192G/Y37F, P199L/A192G/Y37F/T5H, P199L/A192G/Y 37F/T5H/T57S, P199L/A192G/Y37F/T5H/T57S/V175D, A20P/P199L/A192G/Y37F/T5H, A20P/A44M/P199L/A192G/Y37F/T5H, A20P/A44M/T102V/P199L/A192G/Y37F/T5H, Y37W/L62M/N120D/Q165S/C197D, A20P/V39F/P199L, A20P/Y37W/V39F/P199L, A20P/V39F/P199I, A20P/Y37W/V39F/P199I, N15D/A20P/V39F/P199L, N15D/A20P/Y37W/P199L, I30F/Y36W, I30F/F64W, I30F/I106F, Y37W/Y64W, Y37W/I106F, Y64W/I106F, I30F/Y37W/F64W, I30F/Y37W/I106F, I30F/F65W/I106F, Y37W/F64W/I106F, and I30F/Y37W/F64W/I106F; or (f) a polypeptide having an amino acid selected from the group having the amino acid sequence as set forth in SEQ ID NO: 69, SEQ ID NO: 70, SEQ ID NO: 71, SEQ ID NO: 72, SEQ ID NO: 73, SEQ ID NO: 74, and SEQ ID NO: 85, operably linked to a promoter.

In an embodiment of the present disclosure, there is provided a recombinant construct comprising the nucleic acid fragment, said nucleic acid fragment encoding a polypeptide fragment, said polypeptide fragment comprises: (a) a polypeptide having an amino acid sequence selected from the group consisting of SEQ ID NO:14, SEQ ID NO: 16, SEQ ID NO: 18, SEQ ID NO: 20, and SEQ ID NO: 22; or a polypeptide having an amino acid sequence selected from the group consisting of SEQ ID NO: 81, and SEQ ID NO: 83, operably linked to a promoter.

In an embodiment of the present disclosure, there is provided a recombinant construct comprising the nucleic acid fragment, said nucleic acid fragment encoding a polypeptide fragment, said polypeptide fragment comprises: (a) a polypeptide having an amino acid sequence selected from the group consisting of SEQ ID NO: 56, SEQ ID NO: 58, and SEQ ID NO: 60; (b) a polypeptide having an amino acid sequence selected from the group consisting of SEQ ID NO: 52, SEQ ID NO: 54, SEQ ID NO: 62, SEQ ID NO: 64, SEQ ID NO: 66, and SEQ ID NO: 68; or (c) a polypeptide having an amino acid sequence selected from the group consisting of SEQ ID NO: 24, SEQ ID NO: 26, SEQ ID NO: 28, SEQ ID NO: 30, SEQ ID NO: 32, SEQ ID NO: 34, SEQ ID NO: 38, SEQ ID NO: 40, SEQ ID NO: 42, SEQ ID NO: 44, SEQ ID NO: 46, SEQ ID NO: 48, and SEQ ID NO: 50, operably linked to a promoter.

In an embodiment of the present disclosure, there is provided a recombinant construct comprising the nucleic acid fragment, said nucleic acid fragment encoding a polypeptide fragment, said polypeptide fragment comprises a polypeptide having an amino acid sequence selected from the group consisting of SEQ ID NO: 8, SEQ ID NO: 10, SEQ ID NO: 12, SEQ ID NO: 14, SEQ ID NO: 69, SEQ ID NO: 70, SEQ ID NO: 71, SEQ ID NO: 73, SEQ ID NO: 74, SEQ ID NO: 76, SEQ ID NO: 77, SEQ ID NO: 79, SEQ ID NO: 81, SEQ ID NO: 83, and SEQ ID NO: 85, operably linked to a promoter.

In an embodiment of the present disclosure, there is provided a recombinant construct as described herein, wherein the promoter is selected from the group consisting of aprE, tac, T7, Gall/10, AOX1, CMV, and Polyhedrin promoter.

In an embodiment of the present disclosure, there is provided a recombinant construct as described herein, wherein the recombinant construct further comprises: (a) a tpa signal sequence; (b) histidine tag sequence, (c) a linker, (d) HRV3C recognition sequence, or (e) optionally comprising at least one trimerization domain selected the group consisting of human cartilage matrix protein (hCMP), chicken CMP (cCMP), fish cartilage matrix protein (F1CMP), fish isoform 2 cartilage matrix protein (F2-CMP), leucine Zipper with double cysteine (CCIZ), Synthetic trimerization domain (cCMP-IZₘ), foldon, or glycosylated leucine zipper sequence (Gly IZ).

In an embodiment of the present disclosure, there is provided a recombinant construct as described herein, wherein human cartilage matrix protein (hCMP) having an amino acid sequence as set forth in SEQ ID NO: 87, foldon having an amino acid sequence as set forth in SEQ ID NO: 88, chicken CMP (cCMP) having an amino acid sequence as set forth in SEQ ID NO: 89, fish cartilage matrix protein (F1CMP) having an amino acid sequence as set forth in SEQ ID NO: 90, fish isoform 2 cartilage matrix protein (F2-CMP) having an amino acid sequence as set forth in SEQ ID NO: 91, leucine Zipper with double cysteine (CCIZ) having an amino acid sequence as set forth in SEQ ID NO: 92, synthetic trimerization domain (cCMP-IZₘ) having an amino acid sequence as set forth in SEQ ID NO: 93, or glycosylated leucine zipper sequence (Gly IZ) having an amino acid sequence as set forth in SEQ ID NO: 94.

In an embodiment of the present disclosure, there is provided a recombinant construct as described herein wherein the nucleic acid fragment has a nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 75, SEQ ID NO: 78, SEQ ID NO: 80, SEQ ID NO: 82, SEQ ID NO: 84, and SEQ ID NO: 84

In an embodiment of the present disclosure, there is provided a recombinant vector comprising the recombinant construct as described herein.

In an embodiment of the present disclosure, there is provided a recombinant vector as described herein, wherein the recombinant vector is selected from the group consisting of pET vector series, pET15b, pPICZalphaA, pPIC9K, pFastBac1, pcDNA3.4, pcDNA3.1(−), pcDNA3.1(+), and pGEX vector series.

In an embodiment of the present disclosure, there is provided a recombinant host cell comprising the recombinant construct as described herein or the recombinant vector as described herein.

In an embodiment of the present disclosure, there is provided a recombinant host cell as described herein, wherein the recombinant host cell is selected from the group consisting of bacterial cell, yeast cell, insect cell, and mammalian cell, wherein the bacterial cell is Escherichia coli, and wherein the yeast cell is selected from the group consisting of Pichia X33, Pichia GlycoSwitch®, DSMZ 70382, GS115, KM71, KM71H, BG09, GS190, GS200, JC220, JC254, JC227, JC300-JC308, YJN165, and CBS7435, and wherein the insect cell is selected from the group consisting of Expi-Sf9®, Sf9, High Five®, Sf21, and S2, and wherein the mammalian cell is selected from the group consisting of Expi293F® Expi-CHO-S®, CHO-Ki, CHO-S, HEK293F®, CHO^{BC}™, SLIM™, SPOT™, SP2/0, Sp2/0-Ag14, CHO DG44, HEK 293S, HEK 293 Gnt1^{-/-},HEK293-EBNA1, CHOL-NSO, and NSO.

In an embodiment of the present disclosure, there is provided an immunogenic composition comprising a polypeptide fragment as described herein, and a pharmaceutically acceptable carrier.

In an embodiment of the present disclosure, there is provided an immunogenic composition comprising a polypeptide fragment as described herein, and a pharmaceutically acceptable carrier, wherein the pharmaceutically acceptable carrier is selected from the group consisting of at least one adjuvant, and excipients.

In an embodiment of the present disclosure, there is provided an immunogenic composition comprising a polypeptide fragment as described herein, and a pharmaceutically acceptable carrier, wherein the pharmaceutically acceptable carrier is selected from the group consisting of at least one adjuvant selected from the group consisting of an oil-in-water adjuvant, a polymer and water adjuvant, a water-in-oil adjuvant, an aluminum hydroxide adjuvant, and combinations thereof, and excipients. In an exemplary embodiment of the present disclosure, the pharmaceutically acceptable carrier is selected from the group consisting of alhydrogel (aluminium hydroxide adjuvant), Alhydrogel CpG, Addavax (oil-in-water adjuvant), SWE (squalene-in-water emulsion adjuvant), and MF59.

In an embodiment of the present disclosure, there is provided an immunogenic composition comprising a polypeptide fragment comprising a polypeptide having an amino acid sequence selected from the group consisting of SEQ ID NO: 2, SEQ ID NO: 4, SEQ ID NO: 6, SEQ ID NO: 8, SEQ ID NO: 10, and SEQ ID NO: 12, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 8, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 10, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 12, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 69, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 70, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 71, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 73, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 74, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 76, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 77, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 79, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 81, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 83, and a pharmaceutically acceptable carrier.

In an embodiment of the present disclosure, there is provided an immunogenic composition comprising a polypeptide fragment comprising a polypeptide having an amino acid sequence as set forth in SEQ ID NO: 85, and a pharmaceutically acceptable carrier.

In an embodiment of the present disclosure, there is provided an immunogenic composition as described herein, wherein the immunogenic composition comprises a combination of at least two polypeptide fragments having an amino acid sequence selected from the group consisting of SEQ ID NO: 69, and SEQ ID No: 78, and a pharmaceutical acceptable carrier.

In an embodiment of the present disclosure, there is provided an immunogenic composition as described herein, wherein the immunogenic composition comprising a combination of at least two polypeptide fragments having an amino acid sequence selected from the group consisting of SEQ ID NO: 81, and SEQ ID NO: 83, and a pharmaceutically acceptable carrier.

In an embodiment of the present disclosure, there is provided an immunogenic composition as described herein, wherein the pharmaceutically acceptable carrier is selected from the group consisting of selected from the group consisting of at least one adjuvant selected from the group consisting of an oil-in-water adjuvant, a polymer and water adjuvant, a water-in-oil adjuvant, an aluminum hydroxide adjuvant, and combinations thereof, and excipients.

In an embodiment of the present disclosure, there is provided an immunogenic composition as described herein, wherein the immunogenic composition is administered by a method selected from the group consisting of intranasal, subcutaneous, intravenous, intra-arterial, intra-peritoneal, intramuscular, intradermal, oral, dermal, and buccal.

In an embodiment of the present disclosure, there is provided an immunogenic composition as described herein, wherein the immunogenic composition is used in form of a vaccine.

In an embodiment of the present disclosure, there is provided a method for obtaining the immunogenic composition as described herein, wherein the method comprises: (a) culturing the recombinant host cell as described herein under suitable conditions to obtain the polypeptide fragment as described herein; (b) subjecting the polypeptide to purification; and (c) contacting the polypeptide of step (b) with a pharmaceutically acceptable carrier for obtaining the immunogenic composition.

In an embodiment of the present disclosure, there is provided a method for obtaining the immunogenic composition as described herein, wherein the method comprises: (a) culturing the recombinant host cell as described herein under suitable conditions to obtain the polypeptide fragment comprising a polypeptide having an amino acid sequence selected from the group consisting of SEQ ID NO: 2, SEQ ID NO: 4, and SEQ ID NO: 6, SEQ ID NO: 8, SEQ ID NO: 10, and SEQ ID NO: 12; (b) subjecting the polypeptide to purification; and (c) contacting the polypeptide of step (b) with a pharmaceutically acceptable carrier for obtaining the immunogenic composition.

In an embodiment of the present disclosure, there is provided a method for obtaining the immunogenic composition as described herein, wherein the method comprises: (a) culturing the recombinant host cell as described herein under suitable conditions to obtain the polypeptide fragment comprising a polypeptide having an amino acid sequence selected from the group consisting of SEQ ID NO: 8, SEQ ID NO: 10, SEQ ID NO: 12, SEQ ID NO: 14, SEQ ID NO: 69, SEQ ID NO: 70, SEQ ID NO: 71, SEQ ID NO: 73, SEQ ID NO: 74, SEQ ID NO: 76, SEQ ID NO: 77, SEQ ID NO: 79, SEQ ID NO: 81, SEQ ID NO: 83, and SEQ ID NO: 85; (b) subjecting the polypeptide to purification; and (c) contacting the polypeptide of step (b) with a pharmaceutically acceptable carrier for obtaining the immunogenic composition.

In an embodiment of the present disclosure, there is provided a method for obtaining the immunogenic composition as described herein, wherein the recombinant host cell comprising the recombinant construct or the recombinant vector comprises a nucleic acid fragment encoding a polypeptide fragment comprising an amino acid sequence selected from the group consisting of SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 69, SEQ ID NO: 76, SEQ ID NO: 77, SEQ ID NO: 79, SEQ ID NO: 81, SEQ ID NO: 83, and SEQ ID NO: 85, wherein the recombinant host cell is mammalian cell.

In an embodiment of the present disclosure, there is provided a method for obtaining the immunogenic composition as described herein, wherein the recombinant host cell comprising the recombinant construct or the recombinant vector comprises a nucleic acid fragment encoding a polypeptide fragment comprising an amino acid sequence selected from the group consisting of SEQ ID NO: 60, SEQ ID NO: 72, SEQ ID NO: 73, and SEQ ID NO: 74, wherein the recombinant host cell is Pichia pastoris.

In an embodiment of the present disclosure, there is provided a method for obtaining the immunogenic composition as described herein, wherein the recombinant host cell comprising the recombinant construct or the recombinant vector comprises a nucleic acid fragment encoding a polypeptide fragment having an amino acid sequence selected from the group consisting of SEQ ID NO: 56, SEQ ID NO: 58, and wherein the recombinant host cell is insect cells.

In an embodiment of the present disclosure, there is provided a method for obtaining the immunogenic composition as described herein, wherein the recombinant host cell comprising the recombinant construct or the recombinant vector comprises a nucleic acid fragment encoding a polypeptide fragment comprising an amino acid sequence selected from the group consisting of SEQ ID NO: 70, and SEQ ID NO: 71, wherein the recombinant host cell is bacterial cell.

In an embodiment of the present disclosure, there is provided a method for eliciting an immune response in a subject, said method comprising administering the subject a pharmaceutically effective amount of the immunogenic composition as described herein.

In an embodiment of the present disclosure, there is provided a method for eliciting an immune response in a subject as described herein, wherein the immunogenic composition is administered by a method selected from the group consisting of intranasal, subcutaneous, intravenous, intra-arterial, intra-peritoneal, intramuscular, intradermal, oral, dermal, nasal, and inhalation.

In an embodiment of the present disclosure, there is provided a kit comprising the polypeptide as described herein or the immunogenic composition as described herein, and an instruction leaflet.

In an embodiment of the present disclosure, there is provided a polypeptide as described herein, immunogenic composition elicits immune response against severe acute respiratory syndrome coronavirus 2.

In an embodiment of the present disclosure, there is provided a method for preventing or treating a SARS-CoV-2 infection in a subject, said method comprising administering to the subject a pharmaceutically effective amount of the polypeptide fragment as described herein, or the immunogenic composition as described herein.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

### Nucleic Acid and Amino Acid Sequences as Disclosed in the Present Disclosure

The present section exemplifies the present disclosure in form of working examples. The section also lists out the advantage of the present disclosure.

### Sequences Used in the Present Disclosure

SEQ ID NO: 1 depicts the nucleic acid sequence encoding SARS CoV-2 RBD (331-532).

SEQ ID NO: 2 depicts the amino acid sequence of SARS CoV-2 RBD (331-532).

SEQ ID NO: 3 depicts the nucleic acid sequence encoding SARS-CoV-2 RBD N-1 (332-532).

SEQ ID NO: 4 depicts the amino acid sequence of SARS-CoV-2 RBD N-1 (332-532).

SEQ ID NO: 5 depicts the nucleic acid sequence encoding SARS CoV-2 RBD (332-530).

SEQ ID NO: 6 depicts the amino acid sequence of SARS-CoV-2 RBD 3 (332-530).

SEQ ID NO: 7 depicts the nucleic acid sequence encoding mInCV01R (SARS CoV-2 RBD).

SEQ ID NO: 8 depicts the amino acid sequence of mInCVO1R (SARS CoV-2 RBD) having EIS at the N-terminal

SEQ ID NO: 9 depicts the nucleic acid sequence encoding mInCV02R (SARS CoV-2 RBD N-1).

SEQ ID NO: 10 depicts the amino acid sequence of mInCV02R (SARS CoV-2 RBD N-1) having EIS at the N-terminal.

SEQ ID NO: 11 depicts the nucleic acid sequence encoding the mInCV22R SARS-CoV-2 RBD 3 (332-530).

SEQ ID NO: 12 depicts the amino acid sequence of SARS-CoV-2 RBD 3 (332-530).

SEQ ID NO: 13 depicts the nucleic acid sequence encoding mInCV21R (SARS CoV-2 hCMP-RBD).

SEQ ID NO: 14 depicts the amino acid sequence of mInCV21R (SARS CoV-2 hCMP-RBD) having EIS at the N-terminal.

SEQ ID NO: 15 depicts the nucleic acid sequence encoding mInCV26R (SARS CoV-2 RBD with hCMP at C-terminal)

SEQ ID NO: 16 depicts the amino acid sequence of mInCV26R (SARS CoV-2 RBD with hCMP at the C-terminal and EIS at the N-terminal)

SEQ ID NO: 17 depicts the nucleic acid sequence encoding mInCV27R (SARS CoV-2 RBD with Foldon at N-terminal)

SEQ ID NO: 18 depicts the amino acid sequence of mInCV27R (SARS CoV-2 RBD with Foldon having EIS at N terminal)

SEQ ID NO: 19 depicts the nucleic acid sequence encoding mInCV28R (SARS CoV-2 RBD with GlyIZ at N terminal)

SEQ ID NO: 20 depicts the amino acid sequence of mInCV28R (SARS CoV-2 RBD with GlyIZ having EIS at N terminal)

SEQ ID NO: 21 depicts the nucleic acid sequence encoding mInCV29R (SARS CoV-2 RBD with GlyIZ at C-terminal)

SEQ ID NO: 22 depicts the amino acid sequence of mInCV29R (SARS CoV-2 RBD with GlyIZ at C-terminal and EIS at the N-terminal)

SEQ ID NO: 23 depicts the nucleic acid sequence encoding mInCV42R (SARS CoV-2 RBD Chimera Dimer)

SEQ ID NO: 24 depicts the amino acid sequence of mInCV42R (SARS CoV-2 RBD Chimera Dimer)

SEQ ID NO: 25 depicts the nucleic acid sequence encoding mInCV30R (SARS CoV-2 RBD Chimera Dimer with GlyIZ at C-terminal)

SEQ ID NO: 26 depicts the amino acid sequence of mInCV30R (SARS CoV-2 RBD Chimera Dimer with GlyIZ at the C-terminal).

SEQ ID NO: 27 depicts the nucleic acid sequence encoding mInCV31R (SARS CoV-2 RBD chimera dimer with GlyIZ at N-terminal)

SEQ ID NO: 28 depicts the amino acid sequence of mInCV31R (SARS CoV-2 RBD chimera dimer with Gly IZ at N-terminal)

SEQ ID NO: 29 depicts the nucleic acid sequence encoding mInCV32R (SARS CoV-2 RBD chimera dimer with Foldon at C-terminal)

SEQ ID NO: 30 depicts the amino acid sequence of mInCV32R (SARS CoV-2 RBD chimera dimer with Foldon at C-terminal)

SEQ ID NO: 31 depicts the nucleic acid sequence encoding mInCV33R (SARS CoV-2 RBD chimera dimer with Foldon at N-terminal)

SEQ ID NO: 32 depicts the amino acid sequence of mInCV33R (SARS CoV-2 RBD chimera dimer with Foldon at N-terminal)

SEQ ID NO: 33 depicts the nucleic acid sequence encoding mInCV34R (SARS CoV-2 RBD chimera dimer with hCMP at C terminal)

SEQ ID NO: 34 depicts the amino acid sequence of mInCV34R (SARS CoV-2 RBD chimera dimer with hCMP at C-terminal)

SEQ ID NO: 35 depicts the nucleic acid sequence encoding mInCV35R (SARS CoV-2 RBD chimera dimer with hCMP at N-terminal)

SEQ ID NO: 36 depicts the amino acid sequence of mInCV35R (SARS CoV-2 RBD chimera dimer with hCMP at N-terminal)

SEQ ID NO: 37 depicts the nucleic acid sequence encoding mInCV36R (SARS CoV-2 RBD dimer with GlyIZ at C-terminal)

SEQ ID NO: 38 depicts the amino acid sequence of mInCV36R (SARS CoV-2 RBD dimer with GlyIZ at C-terminal)

SEQ ID NO: 39 depicts the nucleic acid sequence encoding mInCV37R (SARS CoV-2 RBD dimer with Gly IZ at N-terminal)

SEQ ID NO: 40 depicts the amino acid sequence of mInCV37R (SARS CoV-2 RBD dimer with Gly IZ at N-terminal)

SEQ ID NO: 41 depicts the nucleic acid sequence encoding mInCV38R (SARS CoV-2 RBD dimer with Foldon at C terminal)

SEQ ID NO: 42 depicts the amino acid sequence of mInCV38R (SARS CoV-2 RBD dimer with Foldon at C-terminal)

SEQ ID NO: 43 depicts the nucleic acid sequence encoding mInCV39R (SARS CoV-2 RBD dimer Foldon at N-terminal)

SEQ ID NO: 44 depicts the amino acid sequence of mInCV39R (SARS CoV-2 RBD dimer with Foldon at N-terminal)

SEQ ID NO: 45 depicts the nucleic acid sequence encoding mInCV40R (SARS CoV-2 RBD dimer with hCMP at C-terminal)

SEQ ID NO: 46 depicts the amino acid sequence of mInCV40R (SARS CoV-2 RBD dimer with hCMP at C-terminal)

SEQ ID NO: 47 depicts the nucleic acid sequence encoding mInCV41R (SARS CoV-2 RBD dimer with hCMP at N-terminal)

SEQ ID NO: 48 depicts the amino acid sequence of mInCV41R (SARS CoV-2 RBD dimer with hCMP at N-terminal)

SEQ ID NO: 49 depicts the nucleic acid sequence encoding mInCV43R (SARS CoV-2 RBD dimer)

SEQ ID NO: 50 depicts the amino acid sequence of mInCV43R (SARS CoV-2 RBD dimer)

SEQ ID NO: 51 depicts the nucleic acid sequence encoding SARS CoV-2 NTD

SEQ ID NO: 52 depicts the amino acid sequence of SARS CoV-2 NTD.

SEQ ID NO: 53 depicts the nucleic acid sequence encoding a fusion polypeptide SARS CoV-2 NTD-RBD (without the linker).

SEQ ID NO: 54 depicts the amino acid sequence of polypeptide SARS CoV-2 NTD-RBD (with a linker GSAGS).

SEQ ID NO: 55 depicts the nucleic acid sequence encoding iInCV01R (SARS CoV-2 RBD)

SEQ ID NO: 56 depicts the amino acid sequence of iInCV01R (SARS CoV-2 RBD)

SEQ ID NO: 57 depicts the nucleic acid sequence encoding iInCV02R (SARS CoV-2 RBD)

SEQ ID NO: 58 depicts the amino acid sequence of iInCV02R (SARS CoV-2 RBD)

SEQ ID NO: 59 depicts the nucleic acid sequence encoding pInCV02R (SARS CoV-2 RBD N-1 (332-532)

SEQ ID NO: 60 depicts the amino acid sequence of pInCV02R (SARS CoV-2 RBD N-1 (332-532).

SEQ ID NO: 61 depicts the nucleic acid sequence encoding mInCV05NR (SARS CoV-2 NTD-RBD)

SEQ ID NO: 62 depicts the amino acid sequence of mInCV05NR (SARS CoV-2 NTD-RBD)

SEQ ID NO: 63 depicts the nucleic acid sequence encoding mInCV07N (SARS CoV-2 NTD)

SEQ ID NO: 64 depicts the amino acid sequence of mInCV07N (SARS CoV-2 NTD)

SEQ ID NO: 65 depicts the nucleic acid sequence encoding pInCV04NR (SARS CoV-2 NTD-RBD)

SEQ ID NO: 66 depicts the amino acid sequence of pInCV04NR (SARS CoV-2 NTD-RBD)

SEQ ID NO: 67 depicts the nucleic acid sequence encoding iInCV03NR (SARS CoV-2 NTD-RBD)

SEQ ID NO: 68 depicts the amino acid sequence of iInCV03NR (SARS CoV-2 NTD-RBD)

SEQ ID NO: 69 depicts the amino acid sequence of DM37

SEQ ID NO: 70 depicts the amino acid sequence of DM47

SEQ ID NO: 71 depicts the amino acid sequence of DM48

SEQ ID NO: 72 depicts the amino acid sequence of pDM48R

SEQ ID NO: 73 depicts the amino acid sequence of pDM49R

SEQ ID NO: 74 depicts the amino acid sequence of pDM49+SA Mutation

SEQ ID NO: 75 depicts the nucleic acid sequence encoding DM37-CHO

SEQ ID NO: 76 depicts the amino acid sequence of DM37-CHO

SEQ ID NO: 77 depicts the amino acid sequence of DM-37a

SEQ ID NO: 78 depicts the nucleic acid sequence encoding DM37-SA

SEQ ID NO: 79 depicts the amino acid sequence of DM37-SA

SEQ ID NO: 80 depicts the nucleic acid sequence encoding hCMP-DM37

SEQ ID NO: 81 depicts the amino acid sequence of hCMP-DM37

SEQ ID NO: 82 depicts the nucleic acid sequence encoding hCMP-DM37SA

SEQ ID NO: 83 depicts the amino acid sequence of hCMP-DM37SA

SEQ ID NO: 84 depicts the nucleic acid sequence encoding mDM46

SEQ ID NO: 85 depicts the amino acid sequence of mDM46

SEQ ID NO: 86 depicts the amino acid sequence of full length (327-527)

SEQ ID NO: 87 depicts the amino acid sequence of hCMP

SEQ ID NO: 88 depicts the amino acid sequence of foldon

SEQ ID NO: 89 depicts the amino acid sequence of Chicken cartilage matrix protein (cCMP)

SEQ ID NO: 90 depicts the amino acid sequence of Fish Cartilage matrix protein (F1CMP)

SEQ ID NO: 91 depicts the amino acid sequence of Fish isoform 2 cartilage matrix protein (F2-CMP)

SEQ ID NO: 92 depicts amino acid sequence of Leucine Zipper with double cysteine (CCIZ)

SEQ ID NO: 93 depicts the amino acid sequence of Synthetic trimerization domain (cCMP-IZₘ)

SEQ ID NO: 94 depicts the amino acid sequence of Glycosylated leucine zipper sequence (Gly IZ)

SEQ ID NO: 95 depicts the amino acid sequence of sequence of mInCV01R (SARS CoV-2 RBD) having tpa signal sequence at the N-terminal.

The amino acid sequence as depicted in SEQ ID NO: 95 comprises tpa signal sequence, RBD residues, additional residues incorporated at the N and C termini, residual HRV3C recognition sequence, sequence removed by digestion.

SEQ ID NO: 96 depicts the amino acid sequence of mInCV02R (SARS CoV-2 RBD N-1).

The amino acid sequence as depicted in SEQ ID NO: 96 comprises tpa signal sequence, RBD residues, additional residues incorporated at the N and C termini, residual HRV3C recognition sequence, sequence removed by digestion

SEQ ID NO: 97 depicts the nucleotide sequence of forward primer.

SEQ ID NO: 98 depicts the nucleotide sequence of reverse primer

SEQ ID NO: 99 depicts the nucleotide sequence of 2019-nCoV_N1-Forward primer

SEQ ID NO: 100 depicts the nucleotide sequence of 2019-nCoV_N1-Reverse primer

SEQ ID NO: 101 depicts the nucleotide sequence of 2019-nCoV_N1 Probe (6-FAM/BHQ-1)

Following types of vaccine candidates (polypeptides) are disclosed in the present disclosure.

SARS-CoV-2 RBD (SEQ ID NO: 2)—This polypeptide version is having the amino acid sequences 331-532 of SARS-CoV-2 RBD. This polypeptide version is also referred to as RBD1.

SARS-CoV-2 RBD (SEQ ID NO: 4)—This polypeptide version is having the amino acid sequences 332-532 of SARS-CoV-2 RBD. This polypeptide version is also referred to as RBD2.

SARS-CoV-2 RBD (SEQ ID NO: 6)—This polypeptide version is having the amino acid sequences 332-530 of SARS-CoV-2 RBD. This polypeptide version is also referred to as RBD3.

mInCV01R (SARS CoV-2 RBD) having EIS at the N-terminal (SEQ ID NO: 8)—This polypeptide version comprises the amino acid sequences 331-532 of SARS-CoV-2 RBD (i.e., RBD1) with EIS at the N-terminal. It can be appreciated that this polypeptide version may further comprise additional amino acid residues (GS; Glycine and Serine) incorporated at the C-terminal. Alternatively, it can also be appreciated that this polypeptide version may further comprise residual HRV3C recognition sequence (LEVLFQ) incorporated at the C-terminal.

mInCV02R (SARS CoV-2 RBD N-1) having EIS at the N-terminal (SEQ ID NO: 10)—This polypeptide version comprises the amino acid sequences 332-532 of SARS-CoV-2 RBD (i.e., RBD2) with EIS at the N-terminal. It can be appreciated that this polypeptide version may further comprise additional amino acid residues (GS; Glycine and Serine) incorporated at the C-terminal. Alternatively, it can also be appreciated that this polypeptide version may further comprise residual HRV3C recognition sequence (LEVLFQ) incorporated at the C-terminal.

mInCV22R (SARS CoV-2 RBD N-2) having EIS at the N-terminal (SEQ ID NO: 12)—This polypeptide version comprises the amino acid sequences 332-530 of SARS-CoV-2 RBD (i.e., RBD3) with EIS at the N-terminal. It can be appreciated that this polypeptide version may further comprise additional amino acid residues (GS; Glycine and Serine) incorporated at the C-terminal. Alternatively, it can also be appreciated that this polypeptide version may further comprise residual HRV3C recognition sequence (LEVLFQ) incorporated at the C-terminal.

### Polypeptides Optimised for Mammalian Cells—

(i) Different versions of SARS-CoV-2 RBD—SEQ ID NO: 8 (331-532), SEQ ID NO: 10 (332-532), SEQ ID NO: 12 (332-530), SEQ ID NO: 14, SEQ ID NO: 16, SEQ ID NO: 18, SEQ ID NO: 20, SEQ ID NO: 22.

(ii) Different versions of Fusion polypeptide comprising NTD and RBD of SARS-CoV-2—SEQ ID NO: 62; NTD domain of SARS-CoV-2—SEQ ID NO: 64.

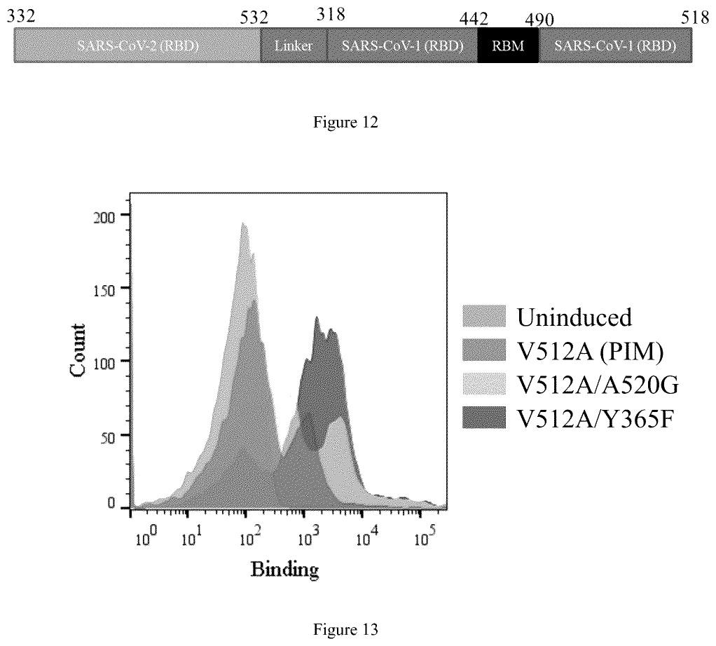

(iv) Different versions of RBD chimera fused with SARS-CoV-2 RBD, the RBD chimera consists of Residues 318-442 and 490-518 from SARS-CoV-1 with an insertion of the Receptor Binding Motif (RBM) of SARS-CoV-2 (residues 454-503 of SARS-CoV-2) inserted between residues 442 and 490 of SARS-CoV-1 (refer to FIG. 12). The idea is to elicit antibodies against the sequence common to RBDs of SARS-CoV-2 and RBD Chimera which is the RBM—SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, and SEQ ID NO: 50.

### Polypeptides Optimised for Insect Cells—

(i) Different versions of SARS-CoV-2 RBD—SEQ ID NO: 58.

(ii) Different versions of Fusion polypeptide comprising NTD and RBD of SARS-CoV-2—SEQ ID NO: 68.

Polypeptides Optimised for Pichia pastoris—

(i) Different versions of SARS-CoV-2 RBD—SEQ ID NO: 60.

(ii) Different versions of Fusion polypeptide comprising NTD and RBD of SARS-CoV-2—SEQ ID NO: 66.

Polypeptide with One or More Mutations: Vaccine Candidates

The present disclosure describes the identification of one more mutation in polypeptide having SEQ ID NO: 2 (331-532; RBD1), or SEQ ID NO: 4 (332-532; RBD2), or SEQ ID NO: 6 (332-530; RBD3). These polypeptides are transiently expressed in different host cells, including, but not limited to mammalian cells, Pichia pastoris, insect cells, and bacterial cells. The present disclosure also describes the identification of one or mutations in polypeptide: SEQ ID NO: 8 (variant of SEQ ID NO: 2), SEQ ID NO: 10 (variant of SEQ ID NO: 4), SEQ ID NO: 12 (variant of SEQ ID NO: 6).

Table 2 and 3 provides the details of various mutant variants of polypeptide having SEQ ID NO:2, SEQ ID NO:4, SEQ ID NO:6, SEQ ID NO:8, SEQ ID NO: 10, and SEQ ID NO: 12.

TABLE 2

| | Amino | | | Positions | | Positions | | Positions |
|---|---|---|---|---|---|---|---|---|
| | acid | | | w.r.t SEQ | | w.r.t SEQ | | w.r.t SEQ |
| | residue | | Positions | ID NO: 8 | Positions | ID NO: 10 | Positions | ID NO: 12 |
| | with | | w.r.t SEQ | (RBD2 with | w.r.t SEQ | (RBD2 with | w.r.t SEQ | (RBD3 with |
| Mutant | respect | Amino acid | ID NO: 2 | EIS at the | ID NO:4 | EIS at the | ID NO: 6 | EIS at the |
| variant | to RBD | Substitution | (RBD1) | N-terminal) | (RBD2) | N-terminal) | (RBD3) | N-terminal) |
| M | 333 | T to H | 3 | 6 | 2 | 5 | 2 | 5 |
| M3 | 348 | A to P | 18 | 21 | 17 | 20 | 17 | 20 |
| M4 | 365 | Y to F | 35 | 38 | 34 | 37 | 34 | 37 |
| M5 | 372 | A to M | 42 | 45 | 41 | 44 | 41 | 44 |
| M6 | 385 | T to S | 55 | 58 | 54 | 57 | 54 | 57 |
| M9 | 430 | T to V | 100 | 103 | 99 | 102 | 99 | 102 |
| M11 | 460 | N to V | 130 | 133 | 129 | 132 | 129 | 132 |
| M12 | 468 | I to V | 138 | 141 | 137 | 140 | 137 | 140 |
| M14 | 520 | A to G | 190 | 193 | 189 | 192 | 189 | 192 |
| M15 | 527 | P to L | 197 | 200 | 196 | 199 | 196 | 199 |
| M17 | 527 | P to T | 197 | 200 | 196 | 199 | 196 | 199 |
| DM 2 | 390 | L to M | 60 | 63 | 59 | 62 | 59 | 62 |
| DM 8 | 527 | P to I | 197 | 200 | 196 | 199 | 196 | 199 |
| DM 9 | 346 | R to T | 16 | 19 | 15 | 18 | 15 | 18 |
| DM 10 | 369 | Y to L | 39 | 42 | 38 | 41 | 38 | 41 |
| DM 11 | 389 | D to E | 59 | 62 | 58 | 61 | 58 | 61 |
| DM 21 | 365 | Y to W | 35 | 38 | 34 | 37 | 34 | 37 |
| DM 24 | 372 | A to T | 42 | 45 | 41 | 44 | 41 | 44 |
| DM 26 | 367 | V to F | 37 | 40 | 36 | 39 | 36 | 39 |
| BLM4 | 477 | S to E | 147 | 150 | 146 | 149 | 146 | 149 |
| BLM5 | 468 | I to T | 138 | 141 | 137 | 140 | 137 | 140 |
| BLM6 | 464 | F to Y | 134 | 137 | 133 | 136 | 133 | 136 |
| BLM7 | 459 | S to Q | 129 | 132 | 128 | 131 | 128 | 131 |
| BLM8 | 434 | I to F | 104 | 107 | 103 | 106 | 103 | 106 |
| BLM9 | 428 | D to N | 98 | 101 | 97 | 100 | 97 | 100 |
| BLM10 | 414 | Q to A | 84 | 87 | 83 | 86 | 83 | 86 |
| BLM11 | 408 | R to D | 78 | 81 | 77 | 80 | 77 | 80 |
| BLM12 | 392 | F to W | 62 | 65 | 61 | 64 | 61 | 64 |
| BLM14 | 385 | T to E | 55 | 58 | 54 | 57 | 54 | 57 |
| BLM15 | 383 | S to D | 53 | 56 | 52 | 55 | 52 | 55 |
| BLM16 | 373 | S to K | 43 | 46 | 42 | 45 | 42 | 45 |
| BLM18 | 358 | I to F | 28 | 31 | 27 | 30 | 27 | 30 |
| BLM19 | 354 | N to E | 24 | 27 | 23 | 26 | 23 | 26 |
| BLM20 | 346 | R to K | 16 | 19 | 15 | 18 | 15 | 18 |
| BLM22 | 337 | P to D | 7 | 10 | 6 | 9 | 6 | 9 |

TABLE 3

| | | | Positions | | Positions | | Positions |
|---|---|---|---|---|---|---|---|
| | Position of | | w.r.t SEQ | | w.r.t SEQ | | w.r.t SEQ |
| | amino acid | Positions | ID NO: 8 | Positions | ID NO: 10 | Positions | ID NO: 12 |
| | residue with | w.r.t SEQ | (RBD1 with | w.r.t SEQ | (RBD2 with | w.r.t SEQ | (RBD3 with |
| Mutant | respect to | ID NO: 2 | EIS at N- | ID NO: 4 | EIS at N- | ID NO: 6 | EIS at N- |
| variant | RBD | (RBD1) | terminal) | (RBD2) | terminal) | (RBD3) | terminal) |
| M18 | 527/528/529/ | 197/198/199/ | 200/201/202/ | 196/197/198/ | 199/200/201/ | 196/197/198/ | 199/200/201/ |
| | 530/532 | 200/202 | 203/205 | 199/201 | 202/204 | 199/201 | 202/204 |
| | P527R/K528R/ | P197R/K198R/ | P200R/K201R/ | P196R/K197R/ | P199R/K200R/ | P196R/K197R/ | P199R/K200R/ |
| | K529V/S530P/ | K199V/S200P/ | K202V/S203P/ | K198V/S199P/ | K201V/S202P/ | K198V/S199P/ | K201V/S202P/ |
| | N532V | N202V | N205V | N201V | N204V | N201V | N204V |
| M20 | 527/365 | 197/35 | 200/38 | 196/34 | 199/37 | 196/34 | 199/37 |
| | P527L/Y365F | P197L/Y35F | P200L/Y38F | P196L/Y34F | P199L/Y37F | P196L/Y34F | P199L/Y37F |
| M21 | 527/520/365 | 197/190/35 | 200/193/38 | 196/189/34 | 199/192/37 | 196/189/34 | 199/192/37 |
| | P527L/A520G/ | P197L/A190G/ | P200L/A193G/ | P196L/A189G/ | P199L/A192G/ | P196L/A189G/ | P199L/A192G/ |
| | Y365F | Y35F | Y38F | Y34F | Y37F | Y34F | Y37F) |
| M22 | 527/520/365/ | 197/190/35/3 | 200/193/38/6 | 196/189/34/2 | 199/192/37/5 | 196/189/34/2 | 199/192/37/5 |
| | 333 | | | | | | |
| | P527L/A520G/ | P197L/A190G/ | P200L/A193G/ | P196L/A189G/ | P199L/A192G/ | P196L/A189G/ | P199L/A192G/ |
| | Y365F/T333H | Y35F/T3H | Y38F/T6H) | Y34F/T2H | Y37F/T5H | Y34F/T2H | Y37F/T5H |
| M24 | 527/520/365/ | 197/190/35/ | 200/193/38/ | 196/189/34/ | 199/192/37/ | 196/189/34/ | 199/192/37/ |
| | 333/385 | 3/55 | 6/58 | 2/54 | 5/57 | 2/54 | 5/57 |
| | P527L/A520G/ | P197L/A190G/ | P200L/A193G/ | P196L/A189G/ | P199L/A192G/ | P196L/A189G/ | P199L/A192G/ |
| | Y365F/T333H/ | Y35F/T3H/ | Y38F/T6H/ | Y34F/T2H/ | Y37F/T5H/T57S | Y34F/T2H/T54S | Y37F/T5H/T57S |
| | T385S | T55S | T58S | T54S | | | |
| M25 | 527/520/365/ | 197/190/35/3/ | 200/193/38/ | 196/189/34/ | 199/192/37/ | 196/189/34/ | 199/192/37/ |
| | 333/385/503 | 55/173 | 6/58/176 | 2/54/172 | 5/57/175 | 2/54/172 | 5/57/175 |
| | P527L/A520G/ | P197L/A190G/ | P200L/A193G/ | P196L/A189G/ | P199L/A192G/ | P196L/A189G/ | P199L/A192G/ |
| | Y365F/T333H/ | Y35F/T3H/ | Y38F/T6H/ | Y34F/T2H/ | Y37F/T5H/ | Y34F/T2H/ | Y37F/T5H/T57S/ |
| | T385S/V503D | T55S/V173D | T58S/V176D | T54S/V172D | T57S/V175D | T54S/V172D | V175D) |
| M26 | 348/527/520/ | 18/197/190/ | 21/200/193/ | 17/196/189/ | 20/199/192/ | 17/196/189/ | 20/199/192/37/5 |
| | 365/333 | 35/3 | 38/6 | 34/2 | 37/5 | 34/2 | |
| | A348P/P527L/ | A18P/P197L/ | A21P/P200L/ | A17P/P196L/ | A20P/P199L/ | A17P/P196L/ | A20P/P199L/A1 |
| | A520G/Y365F/ | A190G/Y35F/ | A193G/Y38F/ | A189G/Y34F/ | A192G/Y37F/ | A189G/Y34F/ | 92G/Y37F/T5H |
| | T333H | T3H | T6H | T2H | T5H | T2H | |
| M27 | 348/372/527/ | 18/42/197/ | 21/45/200/ | 17/41/196/ | 20/44/199/ | 17/41/196/ | 20/44/199/192/3 |
| | 520/365/333 | 190/35/3 | 193/38/6 | 189/34/2 | 192/37/5 | 189/34/2 | 7/5 |
| | A348P/A372M/ | A18P/A42M/ | A21P/A45M/ | A17P/A41M/ | A20P/A44M/ | A17P/A41M/ | A20P/A44M/P1 |
| | P527L/A520G/ | P197L/A190G/ | P200L/A193G/ | P196L/A189G/ | P199L/A192G/ | P196L/A189G/ | 99L/A192G/Y3 |
| | Y365F/T333H | Y35F/T3H | Y38F/T6H | Y34F/T2H | Y37F/T5H | Y34F/T2H | 7F/T5H) |
| M28 | 348/372/430/ | 18/42/100/ | 21/45/103/ | 17/41/99/ | 20/44/102/ | 17/41/99/ | 20/44/102/ |
| | 527/520/365/ | 197/190/35/ | 200/193/38/ | 196/189/34/ | 199/192/37/ | 196/189/34/ | 199/192/37/ |
| | 333 | 3 | 6 | 2 | 5 | 2 | 5 |
| | A348P/A372M/ | A18P/A42M/ | A21P/A45M/ | A17P/A41M/ | A20P/A44M/ | A17P/A41M/ | A20P/A44M/ |
| | T430V/527L/ | T100V/P197L/ | T103V/P200L/ | T99V/P196L/ | T102V/P199L/ | T99V/P196L/ | T102V/P199L/ |
| | 520G/365F/ | A190G/Y35F/ | A193G/Y38F/ | A189G/Y34F/ | A192G/Y37F/ | A189G/Y34F/ | A192G/Y37F/ |
| | 333H | T3H | T6H | T2H | T5H | T2H | T5H |
| DM35 | 365/390/448/ | 35/60/118/ | 38/63/121/ | 34/59/117/ | 37/62/120/ | 34/59/117/ | 37/62/120/165/1 |
| | 493/525 | 163/195 | 166/198 | 162/194 | 165/197 | 162/194 | 97 |
| | Y365W/L390M/ | Y35W/L60M/ | Y38W/L63M/ | Y34W/L59M/ | Y37W/L62M/ | Y34W/L59M/ | Y37W/L62M/N |
| | N448D/Q493S/ | N118D/Q163S/ | N121D/Q166S/ | N117D/Q162S/ | N120D/Q165S/ | N117D/Q162S/ | 120D/Q165S/C1 |
| | C525D | C195D | C198D | C194D | C197D | C194D | 97D |
| DM36 | 348/367/527 | 18/37/197 | 21/40/200 | 17/36/196 | 20/39/199 | 17/36/196 | 20/39/199 |
| | A348P/V367F/ | A18P/V37F/ | A21P/V40F/ | A17P/V36F/ | A20P/V39F/ | A17P/V36F/ | A20P/V39F/ |
| | P527L | P197L | P200L | P196L | P199L | P196L | P199L) |
| DM38 | 348/365/367/ | 18/35/37/ | 21/38/40/ | 17/34/36/ | 20/37/39/ | 17/34/36/ | 20/37/39/ |
| | 527 | 197 | 200 | 196 | 199 | 196 | 199 |
| | A348P/Y365W/ | A18P/Y35W/ | A21P/Y38W/ | A17P/Y34W/ | A20P/Y37W/ | A17P/Y34W/ | A20P/Y37W/V3 |
| | V367F/P527L | V37F/P197L | V40F/P200L | V36F/P196L | V39F/P199L | V36F/P196L | 9F/P199L |
| DM39 | 348/367/527 | 18/37/197 | 21/40/200 | 17/36/196 | 20/39/199 | 17/36/196 | 20/39/199 |
| | A348P/V367F/ | A18P/V37F/ | A21P/V40F/ | A17P/V36F/ | A20P/V39F/ | A17P/V36F/ | A20P/V39F/ |
| | P527I | P197I | P200I | P196I | P199I | P196I | P199I) |
| DM40 | 348/365/367/ | 18/35/37/ | 21/38/40/ | 17/34/36/ | 20/37/39/ | 17/34/36/ | 20/37/39/ |
| | 527 | 197 | 200 | 196 | 199 | 196 | 199 |
| DM42 | A348P/Y365W/ | A18P/Y35W/ | A21P/Y38W/ | A17P/Y34W/ | A20P/Y37W/ | A17P/Y34W/ | A20P/Y37W/ |
| | V367F/P527I | V37F/P197I | V40F/P200I | V36F/P196I | V39F/P199I | V36F/P196I | V39F/P199I |
| | 343/348/367/ | 13/18/37/ | 16/21/40/ | 12/17/36/ | 15/20/39/ | 12/17/36/ | 15/20/39/ |
| | 527 | 197 | 200 | 196 | 199 | 196 | 199 |
| | N343D/A348P/ | N13D/A18P/ | N16D/A21P/ | N12D/A17P/ | N15D/A20P/ | N12D/A17P/ | N15D/A20P/ |
| | V367F/P527L | V37F/P197L | V40F/P200L | V36F/P196L | V39F/P199L | V36F/P196L | V39F/P199L |
| DM43 | 343/348/365/ | 13/18/35/ | 16/21/38/ | 12/17/34/ | 15/20/37/ | 12/17/34/ | 15/20/37/ |
| | 527 | 197 | 200 | 196 | 199 | 196 | 199 |
| | N343D/A348P/ | N13D/A18P/ | N16D/A21P/ | N12D/A17P/ | N15D/A20P/ | N12D/A17P/ | N15D/A20P/ |
| | Y365W/P527L | Y35W/P197L | Y38W/P200L | Y34W/P196L | Y37W/P199L | Y34W/P196L | Y37W/P199L |
| BMM1 | 358/365 | 28/35 | 31/37 | 27/34 | 30/36 | 27/34 | 30/36 |
| | I358F/Y365W | I28F/Y35W | I31F/Y38W | I27F/Y34W | I30F/Y36W | I27F/Y34W | I30F/Y36W |
| BMM2 | 358/392 | 28/62 | 31/65 | 27/61 | 30/64 | 27/61 | 30/64 |
| | I358F/F392W | I28F/F62W | I31F/F65W | I27F/F61W | I30F/F64W | I27F/F61W | I30F/F64W |
| BMM3 | 358/434 | 28/104 | 31/107 | 27/103 | 30/106 | 27/103 | 30/106 |
| | I358F/1434F | I28F/I104F | I31F/I107F | I27F/I103F | I30F/I106F | I27F/I103F | I30F/1106F |
| BMM4 | 365/392 | 35/62 | 38/65 | 34/61 | 37/64 | 34/61 | 37/64 |
| | Y365W/Y392W | Y35W/Y62W | Y38W/Y65W | Y34W/Y61W | Y37W/Y64W | Y34W/Y61W | Y37W/Y64W |
| BMM5 | 365/434 | 35/104 | 38/107 | 34/103 | 37/106 | 34/103 | 37/106 |
| | Y365W/1434F | Y35W/I104F | Y38W/I107F | Y34W/I103F | Y37W/I106F | Y34W/1103F | Y37W/I106F |
| BMM6 | 392/434 | 62/104 | 65/107 | 61/103 | 64/106 | 61/103 | 64/106 |
| | Y392W/I434F | Y62W/I104F | Y65W/1107F | Y61W/I103F | Y64W/I106F | Y61W/I103F | Y64W/I106F |
| BMM7 | 358/365/392 | 28/35/62 | 31/38/65 | 27/34/61 | 30/37/64 | 27/34/61 | 30/37/64 |
| | I358F/Y365W/ | I28F/Y35W/ | I31F/Y38W/ | I27F/Y34W/ | I30F/Y37W/ | I27F/Y34W/ | I30F/Y37W/ |
| | F392W | F62W | F65W | F61W | F64W | F61W) | F64W) |
| BMM8 | 358/365/434 | 28/35/104 | 31/38/107 | 27/34/103 | 30/37/106 | 27/34/103 | 30/37/106 |
| | I358F/Y365W/ | I28F/Y35W/ | I31F/Y38W/ | I27F/Y34W/ | I30F/Y37W/ | I27F/Y34W/ | I30F/Y37W/ |
| | I434F | I104F | I107F | I103F | I106F | I103F | I106F) |
| BMM9 | 358/392/434 | 28/62/104 | 31/65/107 | 27/61/103 | 30/64/106 | 27/61/103 | 30/64/106 |
| | I358F/F392W/ | I28F/F62W/ | I31F/F65W/ | I27F/F61W/ | I30F/F65W/ | I27F/F61W/ | I30F/F65W/ |
| | I434F | I104F | I107F | I102F | I106F | I102F | I106F) |
| BMM10 | 365/392/434 | 35/62/104 | 38/65/107 | 34/61/103 | 37/64/106 | 34/61/103 | 37/64/106 |
| | Y365W/F392W/ | Y35W/F62W/ | Y38W/F65W/ | Y34W/F61W/ | Y37W/F64W/ | Y34W/F61W/ | Y37W/F64W/ |
| | I434F | I104F | I107F | I103F | I106F | I103F | I106F) |
| BMM11 | 358/365/392/ | 28/35/62/ | 31/38/65/ | 27/34/61/ | 30/37/64/ | 27/34/61/ | 30/37/64/ |
| | 434 | 104 | 107 | 103 | 106 | 103 | 106 |
| | I358F/Y365W/ | I28F/Y35W/ | I31F/Y38W/ | I27F/Y34W/ | I30F/Y37W/ | I27F/Y34W/ | I30F/Y37W/ |
| | F392W/I434F | F62W/I104F | F65W/I107F | F62W/1103F | F64W/I106F | F62W/I103F | F64W/I106F |

The other polypeptide versions having mutations have an amino acid sequence as set forth in SEQ ID NO: 69 (DM37), SEQ ID NO: 70 (DM47), SEQ ID NO: 71 (DM48), SEQ ID NO: 72 (pDM48R), SEQ ID NO: 73 (pDM49R), SEQ ID NO: 74 (pDM49+SA MUTATION), SEQ ID NO: 76 (DM37-CHO), SEQ ID NO: 77 (DM-37a), SEQ ID NO: 79 (DM37-SA), SEQ ID NO: 81 (hCMP-DM37), SEQ ID NO: 83 (hCMP-DM37SA), SEQ ID NO: 85 (mDM46).

Table 4 below describes the different features of the recombinant vectors used in the present disclosure.

TABLE 4

| | | Vector | | Promoter, 5′UTR, |
|---|---|---|---|---|
| S. | Name of | Antibiotic | Signal | 3′UTR regions for |
| No. | Plasmid | Resistance* | peptide* | stability |
| 1. | mInCV01R | Kanamycin | tPA | CMV enhancer, CMV |
| | | | | promoter, WPRE |
| | | | | element/bGH poly(A) |
| | | | | signal sequence, intron |
| | | | | A, |
| 2. | mInCV02R | Kanamycin | tPA | CMV enhancer, CMV |
| | | | | promoter, WPRE |
| | | | | element/bGH poly(A) |
| | | | | signal sequence, intron |
| | | | | A, |
| 3. | mInCV05NR | Kanamycin | tPA | CMV enhancer, CMV |
| | | | | promoter, WPRE |
| | | | | element/bGH poly(A) |
| | | | | signal sequence, intron |
| | | | | A, |
| 4. | mInCV07N | Kanamycin | tPA | CMV enhancer, CMV |
| | | | | promoter, WPRE |
| | | | | element/bGH poly(A) |
| | | | | signal sequence, intron |
| | | | | A, |
| 5. | mInCV21R | Kanamycin | tPA | CMV enhancer, CMV |
| | | | | promoter, WPRE |
| | | | | element/bGH poly(A) |
| | | | | signal sequence, intron |
| | | | | A, |
| 6. | iInCV01R | Ampicillin | gp67 | Polyhedron promoter, |
| | | | | gp67 Signal peptide |
| | | | | sequence SV40 poly(A) |
| | | | | signal sequence. |
| 7. | iInCV02R | Ampicillin | gp67 | Polyhedron promoter, |
| | | | | gp67 Signal peptide |
| | | | | sequence, SV40 |
| | | | | poly(A) signal |
| | | | | sequence. |
| 8. | iInCV03NR | Ampicillin | gp67 | Polyhedron promoter, |
| | | | | gp67 Signal peptide |
| | | | | sequence, SV40 |
| | | | | poly(A) signal |
| | | | | sequence. |
| 9. | pInCV02R | Zeocin | AOX1 | AOX1 promoter, |
| | | | | MATalpha signal |
| | | | | sequence, AOX1 |
| | | | | terminator, |
| 10. | pInCV04NR | Zeocin | AOX1 | AOX1 promoter, |
| | | | | MATalpha signal |
| | | | | sequence, AOX1 |
| | | | | terminator, |

### Trimerization Domains

The present disclosure also discloses various trimerization domains that can be fused with the base RBD residues (SEQ ID NO: 2 (331-532), SEQ ID NO 4 (332-532), SEQ ID NO: 6 (331-530), SEQ ID NO: 8 (RBD1 with EIS at the N-terminal), SEQ ID NO: 10 (RBD2 with EIS at the N-terminal), and SEQ ID NO: 12 (RBD3 with EIS at the N-terminal)) to obtain different trimeric derivatives that can be used as suitable vaccine candidates. Different trimerization domains that can be used in the present disclosure are as follows: Human cartilage matrix protein (SEQ ID NO: 87), foldon (SEQ ID NO: 88), chicken cartilage matrix protein (cCMP; SEQ ID NO: 89), fish cartilage matrix protein (F1CMP; SEQ ID NO: 90); fish isoform 2 cartilage matrix protein (F2-CMP; SEQ ID NO: 91), Leucine Zipper with double cysteine (CCIZ; SEQ ID NO: 92), Synthetic trimerization domain (cCMP-IZₘ; SEQ ID NO: 93), Glycosylated leucine zipper sequence (Gly IZ; SEQ ID NO: 94).

Table 5 depicts the position of nucleotide bases of the nucleic acid sequence that encodes various polypeptide versions of the present disclosure.

TABLE 5

| Polypeptide version | Nucleic acid | Nucleotide |
|---|---|---|
| (SEQ ID NO) | (SEQ ID NO) | bases |
| SEQ ID NO: 8 | SEQ ID NO: 7 | 70-684 |
| SEQ ID NO: 10 | SEQ ID NO: 9 | 70-681 |
| SEQ ID NO: 12 | SEQ ID NO: 11 | 70-675 |
| SEQ ID NO: 14 | SEQ ID NO: 13 | 70-852 |
| SEQ ID NO: 16 | SEQ ID NO: 15 | 70-852 |
| SEQ ID NO: 18 | SEQ ID NO: 17 | 70-792 |
| SEQ ID NO: 20 | SEQ ID NO: 19 | 70-831 |
| SEQ ID NO: 22 | SEQ ID NO: 21 | 70-837 |
| SEQ ID NO: 56 | SEQ ID NO: 55 | 115-729 |
| SEQ ID NO: 58 | SEQ ID NO: 57 | 115-726 |
| SEQ ID NO: 60 | SEQ ID NO: 59 | 255-876 |

It is well understood and within the scope of a person skilled in the art to arrive at different variants of the immunogenic composition (vaccine candidate) depending on the host cell in which the recombinant gene is to be expressed for obtaining the vaccine candidate. For clarity, the variants of the vaccine candidate that are optimised for expression in mammalian cells can also be used for preparing the variants for expression in other cells like bacterial, yeast, and insect cells. The present disclosure only discloses a non-specific list of such variants, and many others are possible. Although the present disclosure provides specific examples relating to specific polypeptide fragment used for cloning and expressing it in the host cell by following the methods of cloning a gene of interest, expression of the gene, purification of the protein, and downstream processing. However, it is understood that a person skilled in the art can use any method available in the prior art for obtaining the proteins (vaccine candidate) as described in the present disclosure.

### Example 2

### Selection of the Vaccine Candidate, Cloning, and Purification of the Protein

### Receptor Binding Domain Selection

The receptor binding domain (RBD) residues 331-532 with N-terminal glycosylation site (SEQ ID NO: 2) and 332-532 with N-terminal glycan site deletion of SARS-CoV-2 Spike protein (S) (SEQ ID NO: 4), where the first amino acid is deleted) (accession number YP_009724390.1) were chosen based on SWISS model-based homology-based structure prediction (PDB:2DD8 used as the template). N532 was engineered to be glycosylated by introducing NGS motif at the C-termini of the RBD into both the immunogen sequences. Most of the flexible termini and potential unpaired disulphide residues were eliminated in the receptor engineering strategy. The nucleic acid encoding the entire spike protein of SARS-CoV-2 was accessed from NC045512.2: 21563-25384.

### Cloning

### Mammalian Expression-Based Cloning

The resulting sequence with a HRV-3C precision protease cleavage site linked to 10×Histidine tag by GS linker was mammalian codon optimized and expressed in the pCDNA 3.4 vector under control of a CMV promoter vector containing a tpa signal sequence for efficient secretion in Expi293 cells. The tpa signal sequence is very well known in the art and is coming from the CMV promoter vector.

For the purpose of the present disclosure, two derivatives mInCV01R (having nucleic acid sequence as set forth in SEQ ID NO: 7) (expressing residues 331-532; RBD1) and mInCV02R (having nucleic acid sequence as set forth in SEQ ID NO: 12) (expressing residues 332-532; RBD2) were constructed.

Pichia pastoris (Yeast) Expression-Based Cloning

The resulting sequence with HRV-3C precision protease cleavage site linked to 10×Histidine tag by GS linker was codon optimized for Pichia pastoris expression and cloned into a AOX1 promoter background vector containing a MATalpha signal sequence for efficient secretion. The gene was synthesized and cloned in between EcoRI and NotI in pPICZalphaA by Genscript (USA). The clone were named pInCV01R (331-532) and pInCV02R (332-532) (having nucleic acid sequence as set forth in SEQ ID NO: 59).

### Insect (Baculovirus) Expression-Based Cloning

The resulting sequence with HRV-3C precision protease cleavage site linked to 10×Histidine tag by GS linker was codon optimized for insect cell expression and cloned into a Polyhedron promoter background vector consisting gp67 signal sequence for efficient secretion. The gene was synthesized and cloned in between EcoRI and HindIII in pFASTBac1 by Genscript (USA). The clone was named iInCV01R (331-532) (having nucleic acid sequence as set forth in SEQ ID NO: 55) and iInCV02R (332-532) (having nucleic acid sequence as set forth in SEQ ID NO: 57).

### Purification of Proteins

### Expi293F Protein Purification

Transfections were performed according to the manufacturer's guidelines.

Briefly, one day prior to transfection cells, were passaged at a density of 2×10⁶ cells/ml. On the day of transfection, cells were diluted to 3.0×10⁶ cells/ml. Desired plasmids (1 μg/ml of Expi293F cells) were complexed with ExpiFectamine293 (2.6 μl/ml of Expi293F cells) and transiently transfected into Expi293F cells. Post 16 hr, Enhancer 1 and Enhancer 2 were added according to the manufacturer's protocol. Five days post transfection, culture supernatant was collected, proteins were affinity purified by immobilized metal affinity chromatography (IMAC) using Ni Sepharose 6 Fast flow resin (GE Healthcare). Supernatant was two-fold diluted with 1×PBS (pH 7.4) bound to a column equilibrated with PBS (pH7.4). A ten-column volume wash of PBS (pH7.4), supplemented with 25 mM Immidazole was given. Bound protein was eluted with gradient of 200 mM-500 mM Immidazole in PBS (pH 7.4). The eluted fractions were pooled and dialysed thrice in 3-5 kDa (MWCO) dialysis membrane (40 mm flat width) (Spectrum Labs) against PBS (pH 7.4). Protein concentration was determined by absorbance (A₂₈₀) using the theoretical molar extinction coefficient using the ProtParam tool (ExPASy).

### Pichia Protein Purification

Briefly, 20 μg of pInCV02R vector was linearized with PmeI enzyme by incubating at 37° C. overnight (NEB, R0560). Enzyme was inactivated (65° C., 15 min) prior to PCR purification of the linearized product (Qiagen, Germany). 10 μg of linearized plasmid was transformed into Pichia pastoris X-33 strain by electroporation as per manufactures protocol (Thermo Fisher). Transformants were selected on Zeocin containing YPDS plates (100 μg/ml and 2 mg/ml) (Thermo Fisher Scientific, R25005) up to 3 days at 30° C.

Around 10 random colonies from the YPDS plate (Zeocin 2 mg/ml) were picked and screened for expression by inducing with 1% methanol every 24 hrs. Shake flasks (50 ml) containing 8 ml BMMY media (pH 6.0) each were used for growing the cultures for up to 120 hrs maintained at 30° C., 250 rpm. The expression levels were monitored by dot blot analysis with Anti-his tag antibodies conjugated with a suitable detection signal. The colony showing the highest expression level was then chosen for large scale expression.

The large scale culture was performed in shake flasks by maintaining the same volumetric ratio (flask:media) as the small scale cultures. The expression levels were monitored every 24 hrs using sandwich-ELISA.

The culture was harvested by centrifuging at 4000 g and subsequently filtering through a 0.45μ filter (Sartorius). The supernatant was bound to pre-equilibrated Ni Sepharose 6 Fast flow resin (GE Healthcare). The beads were washed with 1×PBS (pH 7.4) supplemented with 150 mM NaCl and 20 mM Imidazole. Finally, the His tagged RBD protein was eluted in 1×PBS (pH 7.4) supplemented with 150 mM NaCl and 300 mM Imidazole. The eluted fractions were checked for purity on a SDS-PAGE. Following that, appropriate fractions were pooled and dialyzed against 1×PBS (pH7.4) to remove Imidazole.

### ExpiSf9 Protein Purification

Transductions were performed according to the manufacturer's guidelines. Briefly, one day prior to transfection, cells were passaged at a density of 5×10⁶ cells/ml and enhancer was added. On the day of transduction, 1 ml of PO stock virus was used to transduce 50 ml of ExpiSf9 cells. Three days post transfection, culture supernatant was collected, proteins were affinity purified by immobilized metal affinity chromatography (IMAC) using Ni Sepharose 6 Fast flow resin (GE Healthcare). Supernatant was bound to a column equilibrated with PBS (pH7.4). A ten-column volume wash of PBS (pH7.4), supplemented with 25 mM Immidazole was given. Bound protein was eluted with gradient of 200 mM-500 mM Immidazole in PBS (pH 7.4). The eluted fractions were pooled and dialysed thrice in 3-5 kDa (MWCO) dialysis membrane (40 mm flat width) (Spectrum Labs) against PBS (pH 7.4). Protein concentration was determined by absorbance (A₂₈₀) using the theoretical molar extinction coefficient using the ProtParam tool (ExPASy).

### SDS-PAGE and Western Blot Analysis:

SDS-PAGE was performed to estimate the purity and determine the quantity of the proteins (following thermal stability test). SDS-PAGE was performed using an 15% polyacrylamide gel. Protein samples were denatured by boiling with sample buffer containing SDS. Samples were then loaded onto an 15% gel with and without DTT. For western blotting, following SDS-PAGE, proteins were electrophoretically transferred onto an Immobilon-P membrane (Millipore). After transfer, the membrane was blocked with 5% non-fat milk. The membrane was washed with PBST (PBS with 0.05% Tween) and incubated with Mouse anti-His IgG conjugated to HRP (horseradish peroxidase) (Sigma) at 1:5000 dilution. After washing with PBST, an enhanced chemiluminescence (ECL) method was used to develop the blot using HRP substrate and luminol in a 1:1 ratio (Biorad).

### SEC (Size Exclusion Chromatography)

Briefly, a Superdex-200 10/300GL analytical gel filtration column (GE healthcare) equilibrated in PBS (pH 7.4) buffer was utilized for characterizing the changes in the elution volume profile of mInCV01R, mInCV02R. Additionally SEC profiles were obtained for mInCV02R subjected to dialysis, storage at 4° C. overnight, single round freeze thaw, incubated at 37° C. (with and without glycerol) using a ÄKTA Pure chromatography system. The Area under the curve (AUC) was calculated using the peak integrate tool in Evaluation platform for various peaks resultant from the run.

nanoDSF Studies

Equilibrium thermal unfolding experiments of mInCV01R (+/−10×His tag), mInCV02R (+/−10×His tag), iInCV01R (+10×His tag), iInCV02R (+10×His tag), pInCV02R (−10×His tag) were carried out by nanoDSF (Prometheus NT.48) (Chattopadhyay & Varadarajan, 2019). Two independent assays were carried out in duplicate with 10-44 μM of protein in the temperature range of 15-95° C. at 40-80% LED power and initial discovery scan counts (350 nm) ranging between 5000 and 10000.

### SPR—Proteon XPR36 Protein Interaction Array

### SPR-Binding of Immobilized ACE2-hFc/CR3022 to Vaccine Candidates as Analytes

ACE2-hFc and CR3022 neutralizing antibody binding studies with the various vaccine candidates purified from different expression platforms were carried out using the ProteOn XPR36 Protein Interaction Assay V.3.1 from Bio-Rad. Activation of the GLM sensor chip was performed by reaction with EDC (1-Ethyl-3-[3-dimethylaminopropyl] carbodiimide hydrochloride) and sulfo-NHS (N-hydroxysulfosuccinimide) (Sigma). Protein G (Sigma) at 10 μg/ml was coupled in the presence of 10 mM sodium acetate buffer pH 4.5 at 30 μl/min for 300 seconds in various channels. The Response Units for coupling Protein G were monitored till ˜3500-4000RU was immobilized. Finally, the excess sulfo-NHS esters were quenched using 1M ethanolamine. Following this, ACE2 or CR3022 was immobilized on various channels at 5 μg/ml for 100 seconds leaving one channel blank that acts as the reference channel. The Response Units for immobilizing ACE2-hFc and CR3022 were monitored till ˜1000 RU. mInCV01R, mInCV02R (+/−10×His tag), pInCV02R (−10×Histag) were passed at a flow rate of 30 μl/min for 200 seconds over the chip surface, followed by a dissociation step of 600 seconds. A lane without any immobilization was used to monitor non-specific binding. After each kinetic assay, the chip was regenerated in 0.1M Glycine-HCl (pH 2.7) (in the case of ACE2-hFc assay) and 4M MgCl₂ (in case of CR3022 binding assay). The immobilization cycle was repeated prior to each kinetic binding assay in case of ACE2-hFc. Various concentrations of the mInCV01R, mInCV02R (+/−10×His tag), pInCV02R (−10×Histag) (100 nM, 50 nM, 25 nM, 12.5 nM, 6.25 nM) in 1×PBST were used for binding studies. The kinetic parameters were obtained by fitting the data to the simple 1:1 Langmuir interaction model using Proteon Manager.

### SPR-Binding of Immobilized Vaccine Candidates to ACE2-hFc as Analyte

ACE2-hFc binding studies with the various vaccine candidates purified from Expi293F and ExpiSf9 were carried out using the ProteOn XPR36 Protein Interaction Assay V.3.1 from Bio-Rad. Activation of the GLM sensor chip was performed by reaction with EDC (1-Ethyl-3-[3-dimethylaminopropyl] carbodiimide hydrochloride) and sulfo-NHS (N-hydroxysulfosuccinimide) (Sigma).Following this, 10 μg/ml of anti-His monoclonal antibody was coupled in the presence of 10 mM sodium acetate buffer pH 4.0 at 30 μl/min for 100 seconds in various channels, leaving one reference channel blank. The Response Units (RU) for coupling were monitored till ˜3500-4000RU was immobilized. Finally, the excess sulfo-NHS esters were quenched using 1M ethanolamine. C-terminal 10×His tagged vaccine candidates: mInCV01R, mInCV02R (subject to thermal stress, freeze thaw and lyophilization), iInCV01R and iInCV02R were captured onto immobilized anti-His monoclonal antibody at ˜180-320 RU at a flow rate of 30 μl/min. ACE2-hFc was passed as analyte at a flow rate of 30 μl/min for 200 seconds over the chip surface, followed by a dissociation step of 600 seconds. A lane without any immobilization of vaccine candidate was also used to monitor non-specific binding. After each kinetic assay, the chip was regenerated in 4M MgCl₂ and re-immobilized with vaccine candidates. Various concentrations of the ACE2-hFc (100 nM, 50 nM, 25 nM, 12.5 nM, 6.25 nM) in 1×PBST were used for binding studies. The kinetic parameters were obtained by fitting the data to the simple 1:1 Langmuir interaction model using Proteon Manager.

### Limited Proteolysis

Isothermal limited proteolysis assay was carried out for mInCV01R/02R and pInCV02R by at TPCK-Trypsin at 4° C. and 37° C. Briefly, mInCV01R/02R, pInCV02R was dialyzed in autoclaved water (MQ) and reconstituted in the digestion buffer (50 mM Tris, 1 mM CaCl₂) (pH 7.5)). ˜100 μg of mInCV01R/02R and pInCV02R was subject to proteolysis with 2 μg of TPCK-trypsin (TPCK Trypin: Vaccine candidate=1:50) incubated at two different temperatures 4° C. and 37° C. with equal volume of sample drawn at various time points 0, 2, 5, 10, 20, 30 and 60 minutes. The reaction was quenched by instantaneous heat denaturation and analysed by SDS-PAGE.

### Results

### Design of a Recombinant RBD Subunit Vaccine

Receptor binding domain is one of the major targets of neutralizing antibodies on the Spike protein. SARS-CoV-2 is 88% genetically identical to Bat-SARS like coronavirus and the S protein spike of SARS-CoV-2 is 80% identical to its homolog of SARS-CoV-1. The RBD of SARS-CoV-2 shares 74% amino acid sequence identity with RBD of SARS-CoV-1. Therefore, a receptor binding domain subunit vaccine candidate that is least flexible without any unpaired cysteines and retains the major antibody epitopes of neutralizing antibodies would make a suitable vaccine candidate. The RBD residues were designed based on SWISS Model structure-based modelling of SARS-COV-2 sequence prior to availability of any SARS-CoV-2 spike structures and RBD-ACE2 complex structures. The SARS-CoV-2 SWISS modelled RBD has a Cα-Cα RMSD of 0.1 Å compared to SARS-COV-1 RBD used as the template (PDB: 2DD8). The SWISS modelled structure has a Ca-Ca RMSD of 0.7 Å compared following the recent report (PDB: 6M0J). The major structural deviations were localized to the receptor binding motif (RBM) of SARS-CoV-2.

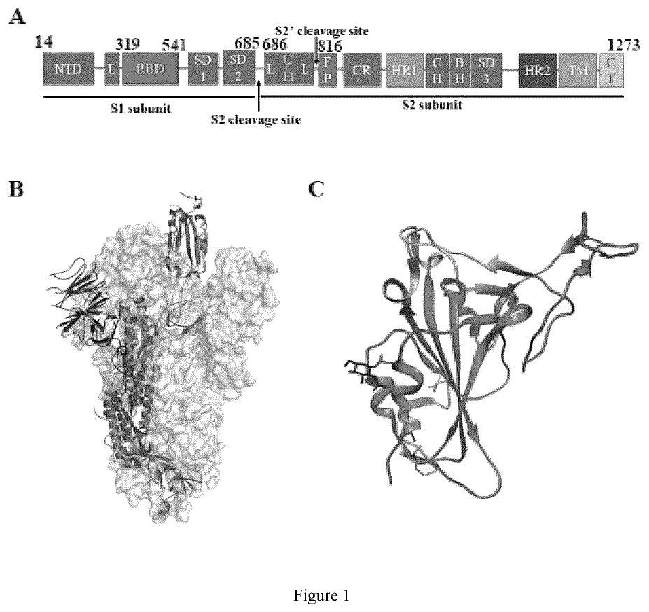

Two RBD sequences were shortlisted consisting of residues 331-532 and 332-532 with addition (nCV01R) and deletion (nCV02R) of native glycan at N331 respectively (FIG. 1C). Both the constructs consist of engineered C-terminal N532 glycan site addition as the addition of glycan will reduce the immune response to the hinge region at the base of RBD construct. The recombinant constructs for mammalian expression are termed mInCV01R (SEQ ID NO: 7), mInCV02R (SEQ ID NO: 9), insect expression constructs are termed iInCV01R (SEQ ID NO: 55), iInCV02R (SEQ ID NO: 57) and Pichia expression construct is termed pInCV02R (SEQ ID NO: 59). In, case of Pichia, the construct was shortlisted based on mammalian expression data screen.

### A High Yielding, Thermo-Functionally Stable and Multiplatform Translatable Recombinant RBD Subunit Vaccine Candidate

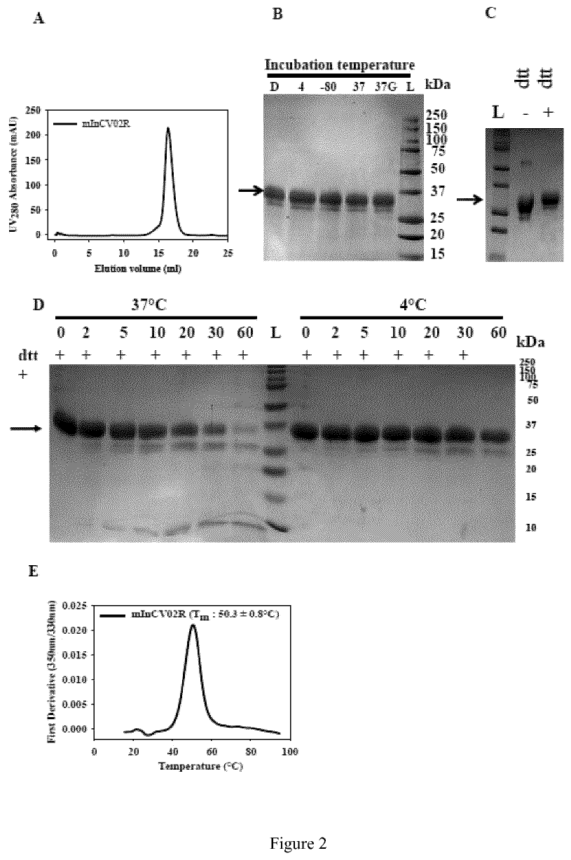

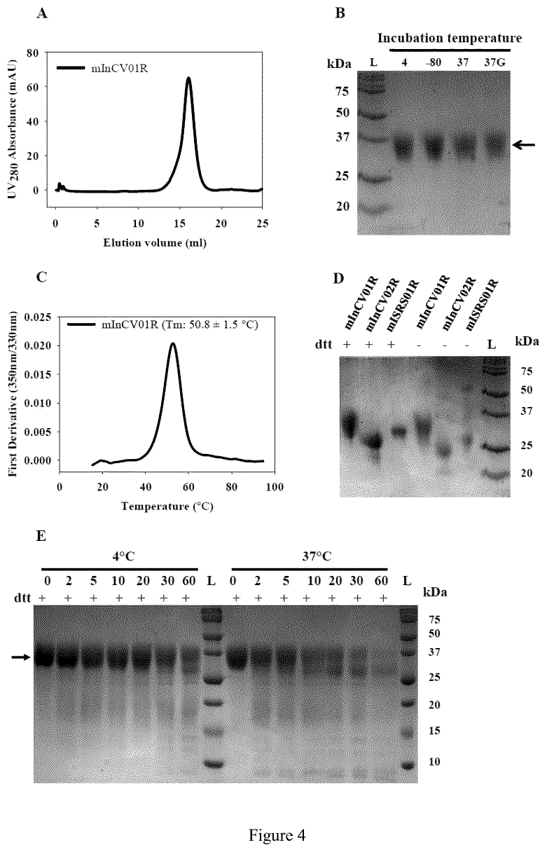

The mammalian expressed mInCV01R (SEQ ID NO: 7) and mInCV02R (SEQ ID NO: 9) were purified by a single step Ni-metal affinity chromatography from transiently transfected Expi293F culture supernatants. Both the constructs were purified to purity as assessed by reducing SDS-PAGE (FIG. 2C). The protein yields were estimated to be ˜32±8.6 mg/L and ˜200±10 mg/L for mInCV01R and mInCV02R respectively. The proteins were confirmed to be predominantly monomeric by SEC and reducing, non-reducing SDS-PAGE (FIG. 2C, and FIG. 4D). The SEC runs highlight the differences in molecular weight of the two constructs owing to the difference in the N terminal glycosylation site. mInCV01R elutes ˜16.0 ml compared to mInCV02R elution volume ˜16.3 ml (FIG. 2A, and FIG. 4A). Thus, it can be concluded that recombinant RBD expressed from mammalian cells mInCV02R was expressed at very high yield and can be purified to homogeneity.

The purified protein expressed from mammalian cells mInCV01R and mInCV02R have an amino acid sequence as set forth in SEQ ID NO: 8, and SEQ ID NO: 10, respectively.

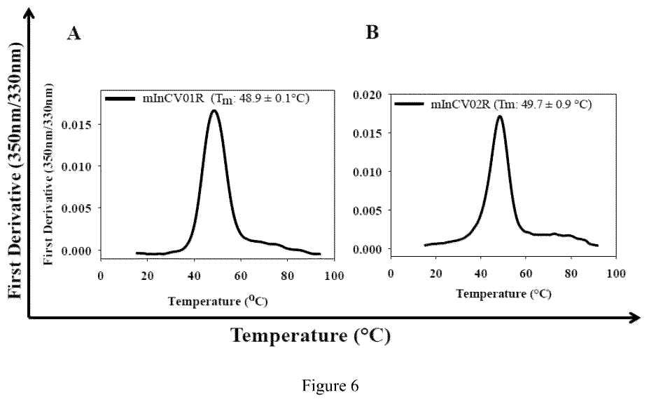

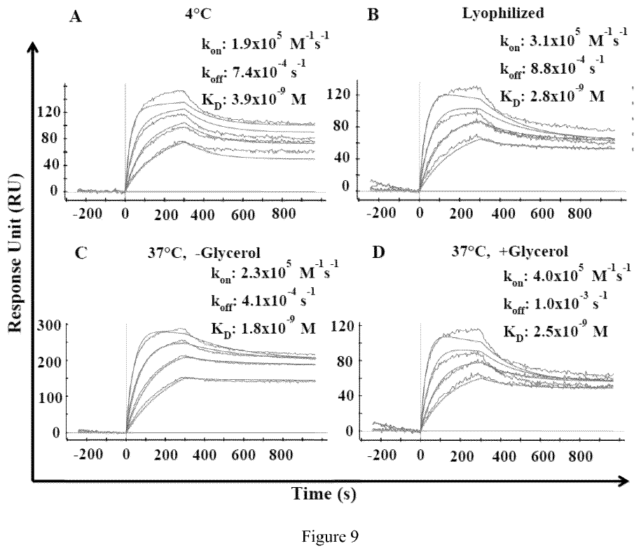

Following the protein purification, thermal stability of the vaccine candidates (SEQ ID NO: 8, SEQ ID NO: 10) was conducted. To this effect, nanoDSF thermal melt studies were conducted and the same revealed nearly similar Tₘ's of Tₘ: 50.8° C. (mInCV01R) and Tₘ: 50.3° C. (mInCV02R) (FIG. 2E, 4C, 5A, 5B). The tagless constructs of mammalian expressed vaccine candidates generated by HRV-3C precision protease digestion also had comparable Tₘ's as the proteins with tag Tₘ: 50.8° C. vs −tag Tₘ: 48.9° C. (mInCV01R) and Tₘ: 50.3° C. vs −tag Tₘ: 49.7° C. (mInCV02R) (FIG. 6A, 6B). In order to confirm the proper folding and dynamic stability of the vaccine candidate limited proteolysis was performed with TPCK-Trypsin. Limited proteolysis revealed that both the vaccine candidate mInCV01R, mInCV02R are stable to trypsin digestion for an hour at 37° C. and greater than an hour at 4° C. It was thus concluded that the vaccine immunogens are well folded and dynamically stable (FIG. 2D, 4E). In order to probe if the immunogens are well folded and display the functional epitopes, SPR binding studies with ACE2 receptor and a known neutralizing antibody CR3022 was performed. It was observed that mInCV01R and mInCV02R bound ACE-2-hFc with similar affinity of ˜3 nM (FIG. 9A, 9B). Additionally, both immunogens bound CR3022 with comparable affinity with mInCV01R bound with K_{D} of 1.3 nM while mInCV02R bound with K_{D} of 16.5 nM (data not shown). Approximately 10-fold change in binding affinity of CR3022 was observed due to distal glycan site N331 in mInCV01R compared to mInCV02R.

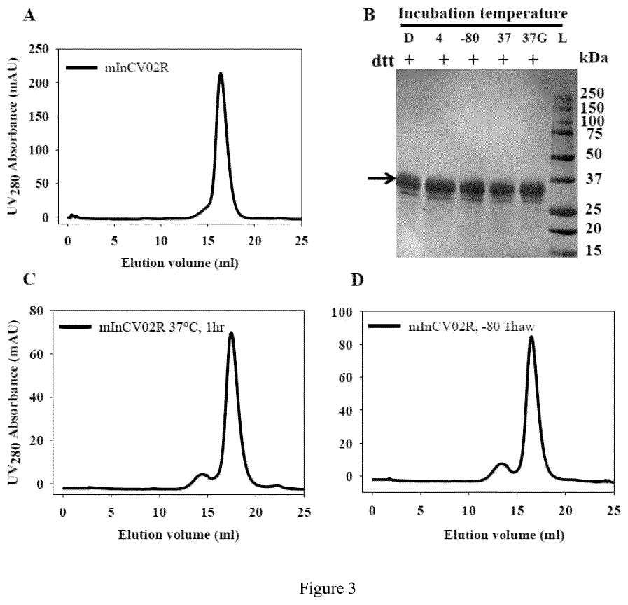

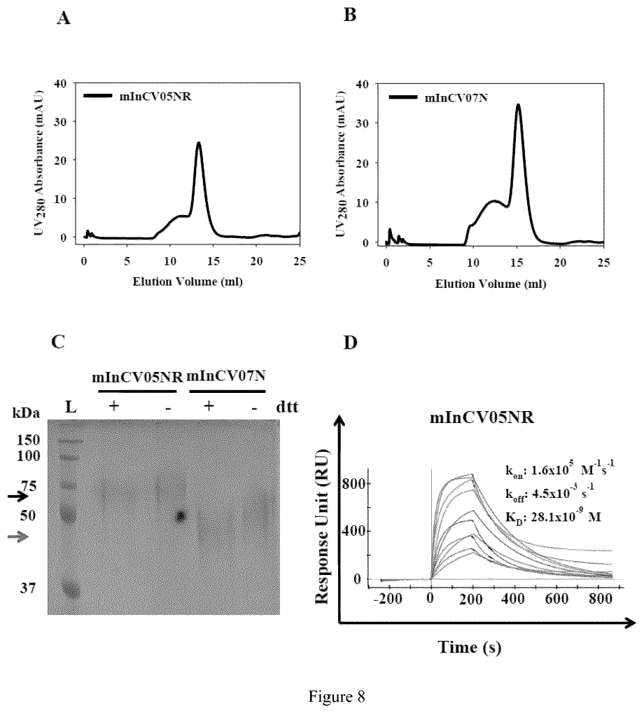

One of the main characteristics of a potential vaccine candidate is the functionality upon storage at 4° C., freeze thaw and subjected to thermal stress due to lack of proper supply chains in low and middle-income countries. In order to test the functionality upon thermal stress SPR binding of mInCV02R to ACE2-hFc was assayed. SPR binding studies reveal that the binding is similar for mInCV02R subjected to storage at 4° C., single freeze thaw, incubation of protein for extended periods of time at 37° C. (overnight storage) and lyophilisation (FIG. 8A-D). Though, SEC runs show a minor peak ˜5 and 8% of the protein tends to aggregate or form higher order complexes upon freeze thaw and storage at 37° C. (without glycerol) for one hour (FIG. 3A, 3C, 3D). Thus, it was concluded that the mInCV02R (SEQ ID NO: 10) is a high expressing, properly folded, dynamically stable, thermo-functionally stable vaccine candidate. It can be contemplated that a person skilled in the art can prepare the recombinant construct mInCV22R (332-530; SEQ ID NO: 13) and can purify the protein (SEQ ID NO: 14) expressed from the mammalian cells mInCV22R in a similar manner as described above for mInCV01R and mInCV02R.

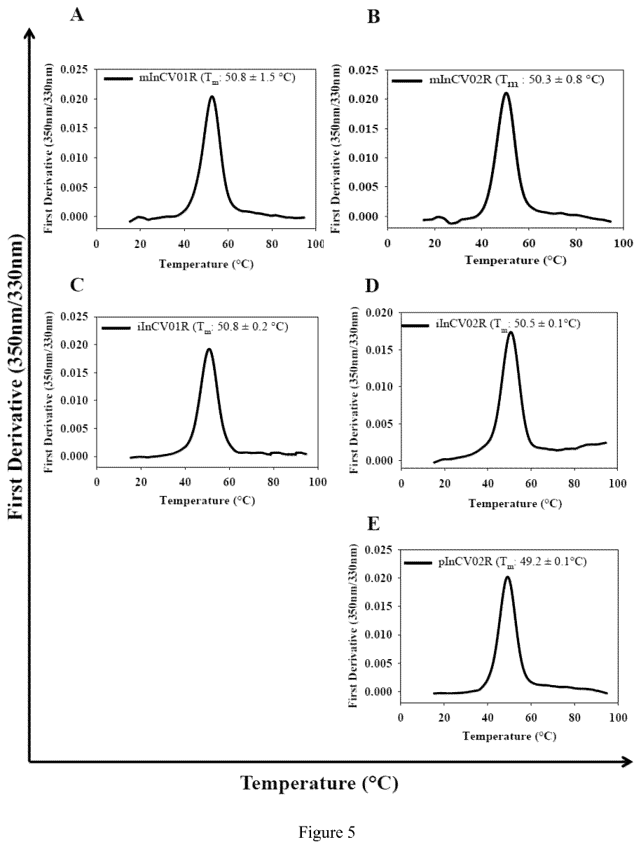

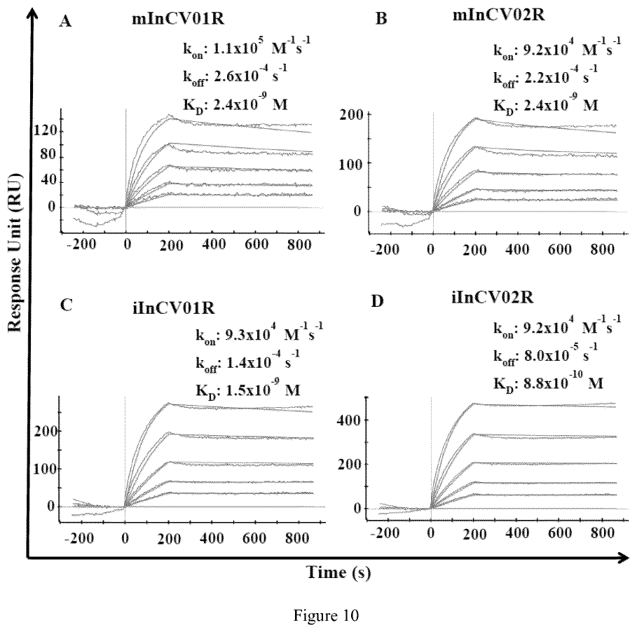

Subsequently, insect cell expression of iInCV01R (SEQ ID NO: 55) and iInCV02R (SEQ ID NO: 57) were attempted. iInCV01R and iInCV02R were purified from transiently transduced ExpiSf9 culture supernatants to purity and homogeneity as assessed by SDS-PAGE. The protein yields were estimated to be ˜15 mg/L and ˜20 mg/L for iInCV01R and iInCV02R respectively. The proteins were confirmed to be predominantly monomeric by reducing and non-reducing SDS-PAGE (data not shown). nanoDSF thermal melt studies of insect expressed vaccine candidates had Tₘ's of 50.8° C. (iInCV01R), 50.5° C. (mInCV02R) similar to mammalian expressed versions 50.8° C. (mInCV01R) and 50.3° C. (mInCV02R) respectively (FIG. 5C, 5D). To test if insect produced immunogens are well folded and functional. SPR binding studies to ACE2-Fc were performed. Both the insect expressed constructs bound with similar affinity to ACE2-hFc as mammalian versions (FIGS. 10 A and B) with an affinity ˜1.5 nM (iInCV01R) (FIG. 10 C) and ˜0.9 nM (iInCV02R) (FIG. 10 D).

The purified protein expressed from insect cell iInCV01R and iInCV02R have an amino acid sequence as set forth in SEQ ID NO: 56, and SEQ ID NO: 58, respectively.

It was concluded that recombinant RBD nCV02R from both mammalian and insect expression platforms (mInCV02R and iInCV02R) expressed at high yield compared to nCV01R (mInCV01R and iInCV01R) and can be purified to homogeneity by a single step affinity chromatography and bound similarly to ACE-hFc.

After down selecting the high expression construct from mammalian and insect expression platforms. The nCV02R Pichia construct (pInCV02R; SEQ ID NO: 59) was expressed and purified from PichiaX-33 from stably integrated gene cassette. An initial screening of selected colonies revealed a highly expressing colony by dot blot and western blot analysis (data not shown). The highest expression colony was further upgraded to large scale culture. The recombinant protein expression of pInCV02R was monitored by an anti-His monoclonal antibody capture and ACE2-hFc probe-based sandwich ELISA. The Pichia protein was purified from culture supernatant to purity as assessed by SDS-PAGE and western blot analysis (data not shown). The purified protein expressed from Pichia construct (pInCV02R) have an amino acid sequence as set forth in SEQ ID NO: 60.

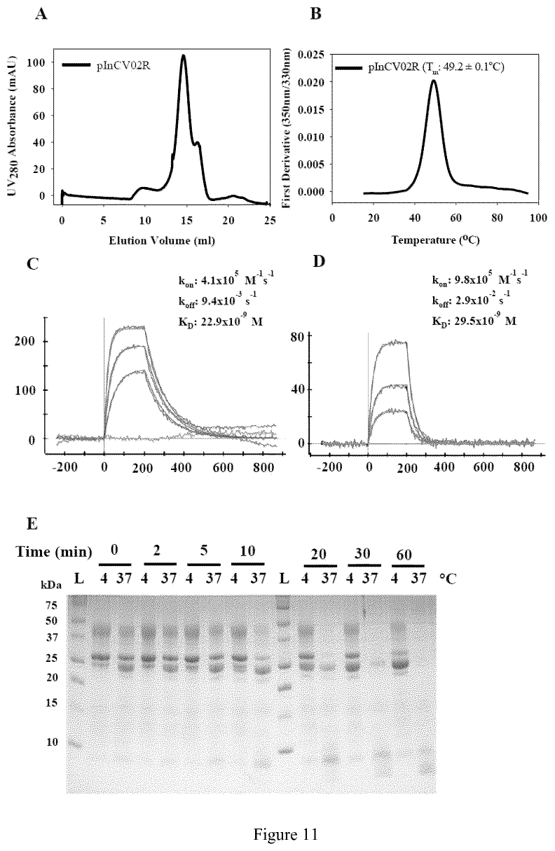

The Pichia protein was observed to be highly glycosylated compared to mammalian or insect-based expression systems. The Pichia protein elutes at ˜14.5 ml (FIG. 11A) before the mammalian equivalent mInCV02R which elutes at ˜16.3 ml. The presence of minor dimeric and lower glycosylated peak fraction at the left peak and right shoulder respectively, was also observed. The thermal stability of the Pichia purified immunogen pInCV02R (Tₘ: 49.2° C.) (FIG. 11B) is similar to mammalian and insect versions expressed versions. This indicates that hyper-glycosylation does not alter the thermal stability of the pInCV02R. Finally, the HRV-3C protease digested tagless pInCV02R was tested for binding to ACE2-hFc and CR3022. The tagless pInCV02R bound with comparable affinity with ACE2-hFc (of ˜23 nM and ˜30 nM) (FIGS. 11C and 11D), said affinity was two-fold lower as compared to the mammalian mInCV02R (˜15 nM).

It was concluded that the vaccine candidate nCV02R is a highly expressing, functional to thermal stress and translatable across different systems for expression and purified to homogeneity in a single affinity purification step. Based on the consistency in expression and stability across multiple platforms, immunization studies with small animals (guinea pigs) was performed with mInCV02R tagless protein.

### Example 3

### Immunization Studies

### Guinea Pig Immunizations

Group of 5, female, Hartley strain guinea pigs, (6-8 weeks old, approximately weighing 300 g) were immunized with 20 μg purified recombinant receptor binding domain of SARS-CoV-2 (mInCV02R; SEQ ID NO: 10) protein diluted in 50 μl phosphate-buffered saline (PBS, pH 7.4), and mixed with 50 μl of AddaVax™ adjuvant (vac-adx-10) (1:1 v/v Antigen: AddaVax™ ratio per animal/dose) (InvivoGen, USA). Immunizations were given by intramuscular injection on Day 0 (prime) and 21 (boost). Blood was collected, and serum isolated on day −2 (pre-bleed), 14 and 35, following the prime and boost immunization, respectively.

### ELISA-Serum Binding Antibody End Point Titers

Briefly, to determine the ELISA end point titers, micro-well plates were coated with immunized vaccine antigen and incubated for two hours at 25° C. (mInCV02R, 4 μg/ml, in 1×PBS, 50 μl/well) under constant shaking (300 rpm) on a MixMate thermomixer (Eppendorf, USA). ACE2-hFc protein coating was used as control for RBD immobilization. Following that, four washes with PBST were given (200 μl/well) and blocked with blocking solution (100 μl, 5% skimmed milk in 1×PBST) and incubated for one hour at 25° C., 300 rpm. Next, Anti-sera (60 μl) starting at 1:100 dilution and four-fold serial dilutions were added and incubated for 1 hour at 25° C., 300 rpm. Three washes with PBST were given (200 μl of PBST/well). Following that, Rabbit raised ALP enzyme conjugated anti-Guinea Pig IgG secondary antibody (diluted 1:5000 in blocking buffer) (50 μl/well) was added and incubated for 1 hour at 25° C., 300 rpm (Sigma-Aldrich, #SAB3700359). Subsequently, four washes were given (200 μl of PBST/well). pNPP liquid substrate (50 μl/well) (pNPP, Sigma-Aldrich, Cat #P7998) was added and plate was incubated for 30 minutes at 25° C., 300 rpm. Finally, the chromogenic signal was measured at 405 nm. The last sera dilution which has a signal above the cut off value (0.02 O.D. at 405 nm) is considered as endpoint titer for ELISA.

### ACE2-hFc Competition ELISA

Briefly, to determine the percent competition of sera targeting the receptor binding motif, micro-well plates were coated with immunized vaccine antigen and incubated overnight at 25° C. (mInCV02R, 4 μg/ml, in 1×PBS, 50 μl/well) under constant shaking (300 rpm) on a MixMate thermomixer (Eppendorf, USA). Ovalbumin (4 μg/ml, in 1×PBS, 50 μl/well) coating was used as negative control for RBD immobilization. Following that, four washes with PBST were given (200 μl/well) and blocked with blocking solution (100 μl, 5% skimmed milk in 1×PBST) and incubated for one hour at 25° C., 300 rpm. Next, Anti-sera (60 μl) starting at 1:10 to 1:1000 dilution were added to sera competition wells and blocking reagent were added to positive control wells and incubated for 1 hour at 25° C., 300 rpm. Three washes with PBST were given (200 μl of PBST/well). An additional blocking was performed for one hour with blocking solution (100 μl) incubated at 25° C., 300 rpm. Following that, ACE2-hFc was added (60 μl at 20 μg/ml) and incubated one hour at 25° C., 300 rpm. Three washes were given (200 μl of PBST/well). Following that, Rabbit raised ALP enzyme conjugated anti-Human IgG secondary antibody (diluted 1:5000 in blocking buffer) (50 μl/well) was added and incubated for 1 hour at 25° C., 300 rpm (Sigma-Aldrich, #SAB3701276). Four washes were given (200 μl of PBST/well). pNPP liquid substrate (50 μl/well) (pNPP, Sigma-Aldrich, Cat #P7998) was added and plate was incubated for 30 minutes at 25° C., 300 rpm. Finally, the chromogenic signal was measured at 405 nm. The percent competition was calculated using the following equation % competition=[Absorbance (Control)−Absorbance (Sera Dilution)]*100/[Absorbance (Control)]. Where, Absorbance (Control) is 405 nm absorbance of ACE2-hFc protein binding to mInCV02R in absence of sera, Absorbance (Sera dilutions) is 405 nm absorbance from wells where sera dilution is incubated with ACE2-hFc protein and mInCV02R.

### Live Virus Neutralization

The guinea pig terminal bleed serum and pre-bleed (negative control) samples were heat inactivated prior to Live virus neutralization assay by incubating at 56° C. for half an hour. SARS-CoV-2 (Isolate: USA-WA1/2020) live virus, Passage 2 was premixed with various dilutions of the serum and incubated at 37° C. for one hour. The incubated premix of virus-serum was added into 96 well plate containing VeroE6 cells and cultured for 48 hours. After completion of incubation, the culture supernatant was collected and analysed for viral RNA by qRT-PCR. The Viral RNA from culture supernatant was extracted according to manufacturer's guidelines.

qRT-PCR was performed using SYBR Green chemistry utilizing the primers targeting SARS-CoV-2 gene on a ThermalCycler. It is understood that a person skilled in the art can arrive at a primer combination based on the genome sequences of SARS-CoV-2 available in the public domain.

### Results

### AddaVax™ Adjuvated RBD Elicits Neutralizing Antibodies in Guinea Pig, Functionally Blocking the Receptor Binding Motif

Animal immunizations in guinea pig was done with mInCV02R tagless protein (SEQ ID NO: 10) adjuvated with AddaVax™. mInCV02R protein prime at day 0 and boost at day 21 regimen was followed with bleed drawn at day −1 (Pre Bleed), day 14 and day 35.

The serum was assayed for binding antibodies by ELISA following prime and boost. The end point titers to self-antigen were 1:100 for pooled sera after the prime and ranged between 1:6400 to 1:102400 after the boost for individual animals (Table 6). It was further tested for competition with ACE2-hFc. Pooled serum samples produced 30% competition at 1:1000 while there is minor variability at higher dilutions in individual animals produced serum competing with ACE2-hFc. G1 and G2 competed 42-46% at 1:1000 serum dilution and two other animals G4 and G5 competed 11% and 5% at 1:1000. However, 60% competing antibodies at serum dilution of 1:500 and 1:100 in G4 and G5 respectively, was observed (Table 7).

TABLE 6
Female Hartley guinea pig (Adda VaxTM adjuvant)

| Animal | | ELISA endpoint titer | | |
|---|---|---|---|---|
| group | | Pool | | |
| with | Animals | Pre- | Primed | Boost |
| adjuvant | identifiers | immune | Sera | Sera |
| Group-1- | G1 | 0^{#} | 100^{#} | 102400 |
| Adda Vax | G2 | | | 25600 |
| | G3 | | | 6400 |
| | G4 | | | 25600 |
| | G5 | | | 6400 |

^{#}Sera pool has been used

TABLE 7
Female Hartley Guinea pig (Adda Vax ™ adjuvant)

| | Competition (%) with ACE2-IgHu | | | | | | |
|---|---|---|---|---|---|---|---|
| | Pre- | | | | | | |
| | immune | Boost | Boost sera of | | | | |
| | Sera | Sera | individual animal numbers | | | | |
| Dilutions | pool | pool | GP-1 | GP-2 | GP-3 | GP-4 | GP-5 |
| 1:10 | 2.7 | 85.3 | 85.9 | 86.7 | 69.3 | 85.8 | 82.8 |
| 1:25 | −0.7 | 85.7 | 86.4 | 86.8 | 56.4 | 84.3 | 78.3 |
| 1:50 | −11.6 | 78.1 | 83.1 | 83.3 | 13.0 | 80.5 | 66.7 |
| 1:75 | 8.6 | 82.7 | 83.7 | 85.3 | 22.9 | 77.8 | 63.2 |
| 1:100 | 9.2 | 81.1 | 83.5 | 84.1 | 8.3 | 73.9 | 55.5 |
| 1:200 | 7.3 | 73.6 | 78.6 | 80.8 | 4.8 | 57.1 | 35.9 |
| 1:500 | 6.0 | 54.0 | 65.6 | 64.1 | −2.0 | 59.8 | 15.0 |
| 1:1000 | 3.0 | 30.3 | 42.6 | 46.2 | −20.6 | 11.5 | 5.5 |

Further, it was tested if the serum neutralizes the live SARS-CoV-2 virus. It was observed that the sera neutralized SARS-CoV-2 with a titer ranging from 1:320-1:1280 (Table 8). This serum neutralization is equivalent to that observed in the mRNA clinical trial in humans by Moderna and better than the ChAdOx1 clinical trial in humans by Oxford trial.

TABLE 8

| Tube | | Endpoint neutralization | |
|---|---|---|---|
| number | Sera Name | titres using CPE | Status |
| 1 | GP-X1-Pre-immune- | Not detected | Negative |
| | sera pool | | |
| 2 | GP-X1-Boost -G1 | 1:320 | Positive |
| 3 | GP-X1-Boost-G2 | 1:1280 | Positive |
| 4 | GP-X1-Boost -G3 | 1:160 | Positive |
| 5 | GP-X1-Boost-G4 | 1:640 | Positive |
| 6 | GP-X1-Boost-G5 | 1:320 | Positive |

### Example 4

### Trimeric Receptor Binding Domain (RBD) of SARS-CoV-2 as a Vaccine Candidate, Cloning, and Purification of the Protein.

Trimeric mRBD Recombinant Construct

The monomeric glycan engineered derivative of the receptor binding domain termed mRBD (residues 332-532 possessing an additional glycosylation site at N532) having an amino acid sequence as set forth in SEQ ID NO: 4 as described in Example 2 was used for preparing the trimeric mRBD recombinant construct.

(a) hCMP-mRBD construct: For the construction of hCMP-mRBD, N-terminal trimerization domain of human cartilage matrix protein (hCMP) (hCMP residues 298-340) (accession number AAA63904) linked by a 14-residue flexible linker (ASSEGTMMRGELKN) derived from the V1 loop of HIV-1 JR-FL gp120, having complete amino acid sequence as set forth in SEQ ID NO: 87, was fused to RBD residues 332-532 (accession number YP_009724390.1; SEQ ID NO: 4) with an engineered glycosylation site (NGS) at N532 followed by an HRV-3C precision protease cleavage site linked to a 10× Histidine tag by a GS linker. The hCMP-mRBD construct reincorporated a glycosylation motif “NIT” at the N-terminal of the mRBD recapitulating the native glycosylation site at N331 in SARS-CoV-2 RBD. This construct is termed as hCMP-mRBD.

(b) mRBD-hCMP construct: The C-terminal fusion of hCMP trimerization domain was obtained by fusing mRBD (residues 332-532; SEQ ID NO: 4) to hCMP (residues 298-340) by a five-residue linker (GSAGS). This construct is defined as mRBD-hCMP.

(c) mRBD-GlyIZ construct: Additionally, the C-terminal fusion of Glycosylated IZ trimerization domain was obtained by fusing mRBD (residues 332-532; SEQ ID NO: 4) to Glycosylated IZ (residues “NGTGRMKQIEDKIENITSKIYNITNEIARIKKLIGNRTAS”; SEQ ID NO: 94) followed by a five-residue linker (GSAGS). This construct is defined as mRBD-GlyIZ.

(d) mRBD-SpyCatcher: For preparing the mRBD-SpyCatcher construct, mRBD (residues 332-532; SEQ ID NO: 4) was fused to SpyCatcher (residues 440-549).

All the four constructs, hCMP-mRBD construct, mRBD-hCMP construct, mRBD-GlyIZ construct, and mRBD-SpyCatcher were fused to a precision protease (HRV-3C) cleavage site linked to a 10× Histidine tag by a GS linker.

It can also be contemplated that a person skilled in the art can fuse mRBD residues (RBD1 (residues 332-532); RBD2 (residues 332-532); or RBD3 (residues 332-530) to other trimerization domains also, such as foldon (SEQ ID NO: 88), chicken cartilage matrix protein (cCMP; SEQ ID NO: 89), fish cartilage matrix protein (F1CMP; SEQ ID NO: 90); fish isoform 2 cartilage matrix protein (F2-CMP; SEQ ID NO: 91), Leucine Zipper with double cysteine (CCIZ; SEQ ID NO: 92), Synthetic trimerization domain (cCMP-IZₘ; SEQ ID NO: 93), in a similar manner like hCMP trimerization domain or Glycosylated IZ trimerization domain is used, in order to arrive at the trimeric mRBD recombinant constructs.

### Cloning

### Mammalian Expression-Based Cloning

These four constructs, hCMP-mRBD construct, mRBD-hCMP construct, mRBD-GlyIZ construct, and mRBD-SpyCatcher were further cloned into the mammalian expression vector pcDNA3.4 under control of a CMV promoter and efficient protein secretion was enabled by the tPA secretion signal peptide sequence. CR3022 antibody heavy and light chain genes were synthesized and subcloned into pcDNA3.4 vector by Genscript (USA). The resulting clones were named hCMP-mRBD (mInCV21R; having a nucleic acid sequence as set forth in SEQ ID NO: 13), mRBD-hCMP (mInCV26R; having a nucleic acid sequence as set forth in SEQ ID NO: 15), mRBD-GlyIZ (mInCV29R; having a nucleic acid sequence as set forth in SEQ ID NO: 21), and mRBD-SpyCatcher, respectively.

Pichia pastoris (Yeast) Expression-Based Cloning

The sequence of the construct hCMP-mRBD construct was codon-optimized for expression in Pichia Pastoris and cloned into the vector pPICZaA containing a MATalpha signal sequence for efficient secretion. The resulting clone was named hCMP-pRBD.

### Purification of Proteins

### Purification of Recombinant Proteins Expressed in Expi293F Cells

mRBD, hCMP-mRBD, mRBD-hCMP, mRBD-GlyIZ, mRBD-SpyCatcher, mSpyCatcher protein was purified from transiently transfected Expi293F cells following manufacturer's guidelines (Gibco, Thermofisher). Briefly, 24 hours prior to transfection, cells were passaged at a density of 2×10⁶ cells/mL into prewarmed Expi293F expression media. On the day of transfection, cells were freshly diluted at a density of 4×10⁶ cells/mL and transiently transfected with the desired plasmids. Plasmid DNA (1 μg per 1 mL of Expi293F cells) was complexed with ExpiFectamine293 and transiently transfected into Expi293F cells. Post 18-20 hr, Enhancer 1 and 2 addition was performed following the manufacturer's protocol. At three days following transfection, spent media was utilized for purification of secreted protein by Ni Sepharose 6 Fast flow affinity chromatography resin (GE Healthcare). PBS (pH 7.4) equilibrated column was bound with two-fold diluted supernatant. Protein bound resin was washed with ten-column volumes of 1×PBS (pH7.4) supplemented with 25 mM imidazole. Bound protein was eluted in a gradient of 200-500 mM imidazole supplemented PBS (pH 7.4). The eluted proteins were dialysed against PBS (pH 7.4) using a dialysis membrane of 3-5 kDa (MWCO) (40 mm flat width) (Spectrum Labs). Protein concentration was determined by absorbance (A280) using NanoDropTM2000c with the theoretical molar extinction coefficient calculated using the ProtParam tool (ExPASy).

Expression and Purification of hCMP-pRBD

The hCMP-pRBD plasmid was linearized with PmeI enzyme (NEB, R0560) prior to transformation. 10 μg of linearized plasmid was used for transformation into Pichia pastoris X-33 strain by electroporation as described in the user manual for Pichia expression by Thermo Fisher Scientific. The transformants were selected by plating on YPDS (YPD Sorbitol) plates with 100 μg/ml and 1 mg/ml Zeocin (Thermo Fisher Scientific, R25005) and incubating the plates at 30° C. for up to 3 days.

Further, 25 colonies from the YPDS plate with 1 mg/ml Zeocin were picked and screened for expression by inducing with 1% methanol every 24 hrs. Culture tubes (15 ml) with 1 ml BMMY media (pH 6.0) each were used for inducing the cultures for up to 120 hrs at 30° C. and 250 rpm. The expression levels were checked using a dot blot analysis with Anti-his tag antibodies conjugated with HRP enzyme. The colony showing the highest expression level was then chosen for large scale expression. The large-scale culture was grown in 2-liter baffled shake flasks with 350 ml volume of culture. The expression levels were monitored every 24 hrs using sandwich-ELISA.

The culture was harvested by centrifugation at 12000 g, and the supernatant was filtered through a 0.45-micron filter. The supernatant was then incubated with Ni Sepharose 6 Fast flow resin (GE Healthcare) for 2 hrs. The beads were washed with 50 column volumes of 1×PBS pH 7.4 supplemented with 20 mM Imidazole. The His tagged protein was then eluted using 1×PBS pH 7.4 supplemented with 300 mM Imidazole. The eluted fractions were assessed for purity on a 12% SDS-PAGE. The appropriate fractions were then pooled and dialyzed against 1×PBS to remove Imidazole.

### Tag Removal

HRV-3C precision protease digestion was performed to remove the C-terminal 10×His tag (Protein: HRV-3C=50:1). HRV-3C digestion was performed for 16 hrs at 4° C. in PBS (pH 7.4). Ni Sepharose 6 Fast flow resin (GE Healthcare) affinity exclusion chromatography was performed to obtain the tagless protein (containing the tag C-terminal sequence: LEVLFQ). The unbound tagless proteins concentration was determined by absorbance (A₂₈₀) using NanoDropTM2000c with the theoretical molar extinction coefficient calculated using the ProtParam tool (ExPASy).

The purified protein expressed from hCMP-mRBD, mRBD-hCMP, mRBD-GlyIZ, having an amino acid sequence as set forth in SEQ ID NO: 14, SEQ ID NO: 16, and SEQ ID NO: 22.

### Generation of Polyclonal Stable Cell Lines

Cell Lines, Media and Growth Conditions for Generation of Polycloncal Stable Lines (COVID-19 Antigen hCMP-mRBD-HRV-Tg)

Flp-In™-293 (Thermo Fisher Scientific, Cat #R75007, Lot #2220695) as well as Flp-In™-CHO (Thermo Fisher Scientific, Cat #R75807, Lot #2127131) adherent cells were used for making COVID-19 antigen hCMP-mRBD-HRV-Tg (a stop codon after ‘Q’ of HRV3C site LEVLFQGP) polyclonal stable cell line. The cell line encoded hCMP-mRBD sequence was thus identical to that obtained after tag removal following HRV3C protease cleavage of protein produced by transient transfection. These engineered cells harbored a single Flp-In™ target site from vector ‘pFRT/lacZeo’ which confers Zeocin resistance. Overall, COVID-19 antigen expressing recombinant cells were engineered using these adherent cells (Flp-In™-293 and, Flp-In™-CHO) which were then allowed to the suspension conditions for the protein production.

### Adherent Cell Culture

Flp-In™-293 and Flp-In™-CHO were cultured either in T25 or T75 EasYFlask, with a TC surface, filter cap (Thermofisher Scientific Cat #156367 and 156499) in a moist 8% CO₂ incubator at 37° C.

The adherent Flp-In™-293 cells were grown in DMEM, high glucose media (Thermo Fisher Scientific Catalog #: 11965118) supplemented with 10% Fetal Bovine Serum (FBS), qualified Brazil (Thermo Fisher Scientific Cat #10270106), 100 U/ml Penicillin Streptomycin (Thermo Scientific Cat #15140122), and 100 μg/ml Zeocin™ Selection Reagent (Thermofisher Scientific Cat #R25001).

The adherent Flp-In™-CHO cells were grown in Ham's F-12 Nutrient Mix media (Thermo Fisher Scientific Catalog #: Cat #11765054) supplemented with 10% FBS, 100 U/ml Penicillin-Streptomycin and 100 μg/ml Zeocin™ Selection Reagent.

### Plasmid and Vector

The Flp-In™ T-REx™ core kit containing pOG44 (Flp recombinase expressing plasmid) and pcDNA5/FRT/TO (donor plasmid for gene of interest) was purchased from Invitrogen USA (Cat #K650001).

The gene of interest ‘hCMP-mRBD-HRV-Tg’ was PCR amplified from hCMP-mRBD pCMV1 vector using HindIII site containing forward primer (5′-TATATAAGCTTCTGCAGTCACCGTCCTTAGATC-3′; SEQ ID NO: 97) and XhoI site-containing reverse primer (5′ TATATCTCGAGTCACTGGAACAGCACCTCCAGGGAGCC-3′; SEQ ID NO: 98).

The amplified PCR product was digested with HindIII and XhoI and subcloned into pcDNA5/FRT/TO restricted with the above two enzymes. The clone was confirmed by sequencing.

### Generation of Adherent Polyclonal Flp-In Stable Lines

T25 flasks (5 ml media) having either adherent Flp-In™-293 or Flp-In™-CHO cells (˜80% confluent) were co-transfected with pOG44 (10 μg) and hCMP-mRBD-HRV-Tg-pcDNA5/FRT/TO (5 μg) plasmid DNA using 35 μg of Lipofectamine™ 2000 Transfection Reagent (Thermo Fisher Scientific, Cat #11668030) in serum free media as per the manufacturer instruction for 4 hrs. After 4 hrs, the media was replaced with serum containing media. The cells were incubated for 16 hrs and then trypsinized using 1 ml of 1×-Tryple express enzyme (Thermofisher Scientific, Cat #12604021) and seeded to a T75 flask containing 25 ml of desired media and incubated for further 24 hrs for FLP recombination. After 24h the media was replaced with fresh media having Hygromycin 100 μg/ml (Thermofisher Scientific Cat #10687010) for Flp-In™-293 and 750 μg/ml for Flp-In™-CHO cells. Hygromycin resistant foci were observed after 3 days of selection. Media containing the desired amount of Hygromycin was changed after every 5 days mentioned above. After 18 days in case of Flp-In™-293 and 14 days in case of Flp-In™-CHO, the recombinant hygromycin resistant cells reached to 100% confluency. The secretion of the protein of interest (hCMP-mRBD-HRV-Tg) was confirmed from cell free media using western blotting with polyclonal Guinea pig sera against the same antigen. The confirmed polyclonal cells were frozen in liquid N2 for long term storage. The T75-flask grown polyclonal cells were adapted for shake flask suspension culture and used for protein production.

### Shake Flask Suspension Cell Culture and Protein Production

The suspension cells were grown in 125 or 250-ml Nalgene™ single-use PETG Erlenmeyer flasks with plain bottom and vented closure (Thermofisher Scientific Cat #4115-0125 or 41150250) at 125 rpm with moist 8% CO₂ incubator at 37° C. or as specifically mentioned.

The stable adherent recombinant Flp-In™-293 cells were first trypsinized from the T75 flask and then grown in a suspension flask after adapting them to FreeStyle™ 293 Expression Medium (Thermofisher Scientific Cat #12338018) supplemented with 2% FBS and 50 μg/ml Hygromycin B for ˜6 generations (two passages, doubling time=24h). Approximately 300 million cells were then seeded to 100 ml serum free FreeStyle™ 293 Expression medium for protein production for 3 days. After 3 days, the media was used for protein purification. Approximately 300 million cells were grown further in 100 ml media for 6 days under identical conditions and used again for protein purification with >95% cell viability.

The stable adherent recombinant Flp-In™-CHO cells were first trypsinized from a T75 flask and then grown in a suspension flask for direct adaptation to PowerCHO™ 2 Serum-free Chemically Defined Medium (Lonza, Cat #12-771Q) supplemented with 8 mM L-Glutamine (Thermo Fisher Scientific, Cat #25-030-081) with 50 μg/ml Hygromycin B. First cells were grown for approximately 8 generations (two passages, doubling time=24h) at 37° C. till ˜3 million per ml density. Approximately 300 million cells were then seeded in 100 ml medium for protein production for 3 days at 32° C. After 3 days the media was harvested for protein purification. The approximately 300 million cells were grown further in 100 ml media for 6 days under identical condition and media used for protein purification with >95% cell viability.

### Tagless Protein Purification

The spent media from stable hCMP-mRBD-HRV-Tg-Flp-In™-293 or Flp-In™-CHO grown cells contained the expressed protein. Protein was purified using anion exchange chromatography. 100 ml cell free media was first dialyzed against 30 mM Tris-HCl buffer pH 8.4 overnight at 4° C. using cellulose membrane dialysis tubing (10 kDa molecular weight cutoff, Sigma, Cat #D9527-100FT). 2 mL Q Sepharose™ Fast Flow beads (GE Healthcare, Cat #17-0510-01) were equilibrated with 30 mM Tris-HCl pH 8.4 and incubated for 1 hr at 4° C. with the dialyzed sample. Protein elution was performed with a step gradient of 30 mM Tris-HCl pH 8.4. containing 20-500 mM NaCl. The fractions were analyzed on a 10% SDS-PAGE gel and the pure fractions were pooled and further dialyzed against 1×-PBS buffer pH 7.4, overnight. The pure protein was analyzed on 10% oxidizing as well as reducing SDS PAGE for homogeneity and purity. Size exclusion chromatography utilizing Superose 6 10/300 Increase GL column with 1×PBS as running buffer at a flow rate of 0.5 mL/min on an ÄktaPure (GE) was performed to determine protein aggregation state.

### SDS-PAGE Analysis

Protein purity was estimated by denaturing PAGE. Samples were denatured in SDS containing sample buffer by boiling in reducing (with 3-mercaptoethanol) or non-reducing (without 3-mercaptoethanol) conditions.

### Size Exclusion Chromatography (SEC) and SEC-MALS

SEC profiles were obtained in 1×PBS buffer equilibrated analytical gel filtration Superdex-200 10/300GL column (GE healthcare) on an Äkta pure chromatography system. The peak area under the curve (AUC) was determined in the Evaluation platform using the peak integrate tool.

For SEC-MALS (multi angle light scattering), a PBS (pH 7.4) buffer equilibrated analytical Superdex-200 10/300GL gel filtration column (GE healthcare) on a SHIMADZU HPLC was utilized to resolve hCMP-mRBD purified protein. hCMP purified protein has an amino acid sequence as set forth in SEQ ID NO: 14. Gel filtration resolved protein peaks were subjected to in-line refractive index (WATERS corp.) and MALS (mini DAWN TREOS, Wyatt Technology corp.) detection for molar mass determination. The acquired data from UV, MALS and RI were analysed using ASTRA™ software (Wyatt Technology).

nanoDSF Thermal Melt Studies

Equilibrium thermal unfolding of hCMP-mRBD (−10×His tag) protein, before or after thermal stress was carried out using a nanoDSF (Prometheus NT.48) (Chattopadhyay & Varadarajan, 2019). Two independent measurements were carried out in duplicate with 2-4 μM of protein in the temperature range of 15-95° C. at 100% LED power and initial discovery scan counts (350 nm) ranging between 5000 and 10000. In all cases, when lyophilized protein was used, it was reconstituted in water, prior to DSF.

### Negative Staining Sample Preparation and Visualization by Transmission Electron Microscope

For visualization by a Transmission Electron Microscope, the sample was prepared by a conventional negative staining method. Briefly, the carbon-coated copper grid was glow discharged for 20 seconds at 20 mA using Quorum GlowQube. Around 3.5 μl of hCMP-mRBD sample (0.1 mg/ml) was added to the freshly glow discharged carbon-coated copper grid for 1 minute. The extra sample was blotted out. Negative staining was performed using freshly prepared 1% Uranyl Acetate solution for 20 seconds and the grid was air-dried before TEM imaging. The negatively stained sample was visualized at room temperature using a Tecnai T12 electron microscope equipped with a Tungsten filament operated at 120 kV. Images were recorded using a side-mounted Olympus VELITA (2K and 2K) CCD camera at a calibrated 3.54 Å/pixel.

### Reference-Free 2D Classification Using Single-Particle Analysis

The evaluation of micrographs was done with EMAN 2.1. Around 6600 particles were picked manually and extracted using e2boxer.py in EMAN2.1 software. Reference free 2D classification of different projections of particle were calculated using simple_prime2D of SIMPLE 2.1 software (Reboul, Cyril F., et al. “Single-particle cryo-EM—Improved ab initio 3D reconstruction with SIMPLE/PRIME.” Protein Science 27.1 (2018): 51-61).

SPR-Binding of hCMP-mRBD (Vaccine Candidate) Analyte to Immobilized ACE2-hFc/CR3022

hCMP-mRBD protein kinetic binding studies to ACE2-hFc and CR3022 antibody were performed on a ProteOn XPR36 Protein Interaction Array V.3.1 (Bio-Rad). The GLM sensor chip was activated with sulfo-NHS and EDC (Sigma) reaction. Protein G (Sigma) was covalently coupled following activation. Approximately 3500-4000 RU of Protein G (10 μg/mL) was coupled in 10 mM sodium acetate buffer pH 4.5 at a flow rate of 30 l/min for 300 seconds in desired channels. Finally, 1M ethanolamine was used to quench the excess sulfo-NHS esters. Following quenching, ligand immobilization was carried out at a flow rate of 30 l/min for 100 seconds. ACE2-hFc or CR3022 were immobilized at ˜800 RU on desired channels excluding a single blank channel that acts as the reference channel. hCMP-mRBD analyte interaction with ligands was monitored by passing over the chip at a flow rate of 30 l/min for 200 seconds, and the subsequent dissociation phase was monitored for 600 seconds. An empty lane without ligand immobilization was utilized for measuring non-specific binding. Following each kinetic assay, regeneration was carried out with 0.1 M Glycine-HCl (pH 2.7). The ligand immobilization cycle was repeated prior to each kinetic assay. Various concentrations of the hCMP-mRBD (−10×His tag) (100 nM, 50 nM, 25 nM, 12.5 nM, 6.25 nM) in 1×PBST were used for binding studies. The kinetic parameters were obtained by fitting the data to a simple 1:1 Langmuir interaction model using Proteon Manager.

SPR-Binding of Thermal Stress Subjected hCMP-mRBD Analyte to Immobilized ACE2-hFc

Lyophilized protein or protein in 1×PBS (0.2 mg/mL) was subjected to transient thermal incubation at the desired temperature in a thermal cycler for ninety or sixty minutes, respectively. Post thermal incubation, binding response was assessed at 100 nM analyte concentration by SPR as mentioned above.

### Results

### Design of a Recombinant Trimeric RBDs (Vaccine Candidate) of SARS-CoV-2

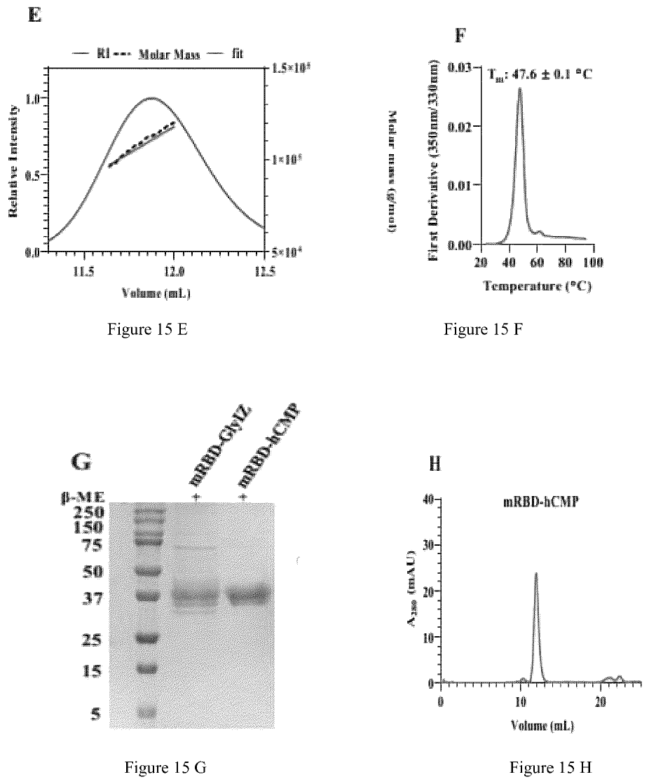

Since the oligomerization of native antigens can induce higher titers of binding and neutralizing antibodies, therefore, mRBD protein (SEQ ID NO: 4) was fused to the disulfide linked trimerization domain derived from human cartilage matrix protein (hCMP) (residues 298-340). RBD fused to the hCMP trimerization domain (residues 298-340), would elicit higher neutralizing antibody titers relative to the corresponding monomer. For designing trimeric mRBDs, the RBD (residues 332-532) from the closed state of the Spike-2P (PDB 6VXX) aligned coaxially with the hCMP trimerization domain were utilized. Referring to FIG. 15A, The N termini of mRBD are labelled as 1332 and C-termini of the hCMP trimerization domain are labelled as V340. The N, C termini Cα's form vertices of equilateral triangles. The N-terminal plane of RBD (I332) was separated from the C-terminal plane (V340) of the hCMP trimerization domain by ˜22.1 Å to avoid steric clashes. The distance between the hCMP C-terminus residue 340 and RBD N-terminus residue 332 was approximately 39.0 Ain the modeled structure and are connected by a 14-residue long linker.

Thus, the trimeric hCMP-mRBD design consisted of the N-terminal hCMP trimeric coiled coil domain (residues 298-340) fused to the 1332 residue of mRBD by the 14-residue long linker, followed by the cleavable His tag sequence as depicted in FIG. 15B. The hCMP trimerization domain leads to formation of covalently stabilized trimers crosslinked by interchain disulfides in the hCMP domain. The construct design is termed as hCMP-mRBD (having nucleic acid sequence as set forth in SEQ ID NO: 13) and hCMP-pRBD, where the “m” and “p” signifies expression in mammalian or Pichia pastoris cells, respectively.

Further, trimeric RBD constructs (residues 332-532) were designed by fusing hCMP and glycosylated IZ synthetic trimerization domains at the C-terminus of RBD, to obtain mRBD-hCMP construct (having nucleic acid sequence as set forth in SEQ ID NO: 15) and mRBD-GlyIZ construct (having nucleic acid sequence as set forth in SEQ ID NO: 21), respectively (FIG. 15 B). GlyIZ is a glycosylated version of the synthetic trimerization domain IZ. The glycosylation results in immunosilencing of the otherwise highly immunogenic IZ sequence. Additionally, mRBD-SpyCatcher construct was constructed by fusion of SpyCatcher to the C-terminus of the mRBD. These fusion constructs were expressed from transiently transfected mammalian cell culture.

Moreover, a dodecameric self-assembling nanoparticle (MsDPS2) from Mycobacterium smegmatis was fused to SpyTag by a 15 residue linker to aid in the complexation of nanoparticle with mRBD-SpyCatcher (FIG. 15 B).

hCMP-mRBD (Vaccine Candidate) Forms Homogenous, Thermotolerant Trimers.

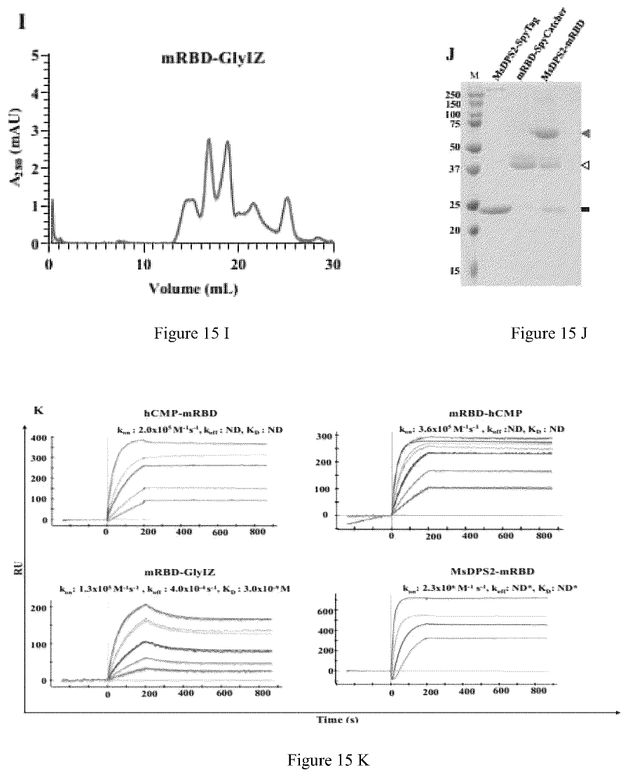

hCMP-mRBD was first expressed by transient transfection in Expi293F suspension cells, followed by single step metal affinity chromatography (Ni-NTA) and tag cleavage. The purified protein was observed to be pure and trimeric by reducing and non-reducing SDS-PAGE, as depicted in FIG. 15C, and FIG. 15D. The protein exists as a homogenous trimer in solution and the molar mass determined by SEC-MALS was 110±10 kDa, which is consistent with the presence of nine glycosylation sites in the trimer (FIG. 15C, FIG. 15E). Negative stain EM analysis confirmed the trilobed arrangement of RBD structure (FIG. 16). It can be inferred from FIG. 16 that the purified hCMP-mRBD protein (SEQ ID NO: 14) is monodisperse and forms a stable trimer.

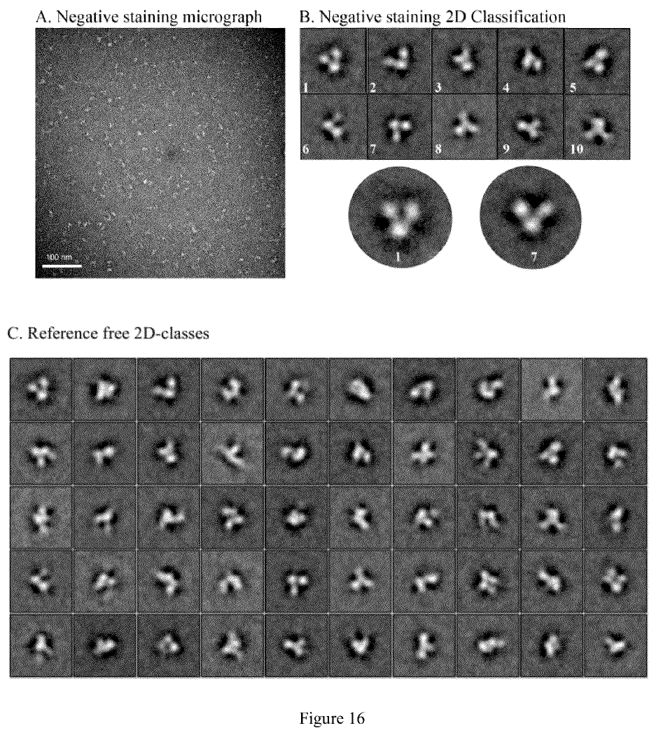

Further, referring to FIG. 15F, it can be observed that the Trimeric hCMP-mRBD was observed to have comparable thermal stability (Tₘ: 47.6° C.) as monomeric mRBD (Tₘ: 50.3° C.). Moreover, from the SPR binding studies conducted with hCMP-mRBD, mRBD-hCMP, mRBD-GlyIZ and SEC purified complex MsDPS2-mRBD to CR3022, it was observed that both Trimeric hCMP-mRBD and monomeric mRBD bound its cognate receptor ACE2 and a SARS-CoV-1 neutralizing antibody CR3022 with very high affinity (K_{D}<1 nM) and negligible dissociation, as depicted in FIG. 15K and FIG. 17.

Similar to hCMP-mRBD construct, the fusion constructs mRBD-hCMP and mRBD-GlyIZ were purified from transiently transfected Expi293F cells. mRBD-GlyIZ was observed to be more heterogeneous compared to hCMP-mRBD and mRBD-hCMP (FIG. 15 C, FIG. 15G, FIG. 15H, FIG. 15I). Referring to FIG. 15K and FIG. 17, mRBD-hCMP showed negligible dissociation and bound its cognate receptor ACE2 and a SARS-CoV-1 neutralizing antibody CR3022 similar to hCMP-mRBD. It can also be observed that mRBD-GlyIZ bound ACE2 and CR3022 with a K_{D} of 3-5 nM.

mRBD-SpyCatcher and MsDPS2-SpyTag were complexed in the ratio 1:3, and the formation of MsDPS2-mRBD nanoparticle conjugate was confirmed by SDS-PAG. Further, the nanoparticulate conjugate was purified by SEC (FIG. 15J). The SEC purified nanoparticulate mRBD bound its cognate receptor ACE2 and a SARS-CoV-1 neutralizing antibody CR3022 with high kₒₙ (>10⁶ M⁻¹s⁻¹) and negligible k_{off}, indicating the formation of a functional MsDPS2-mRBD nanoparticle (FIG. 15K, and FIG. 17).

### Thermal Stress

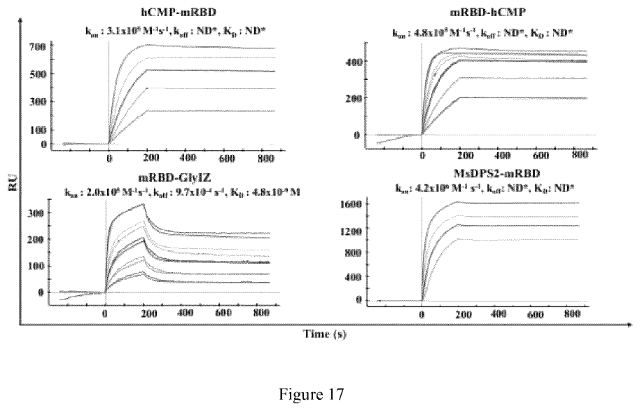

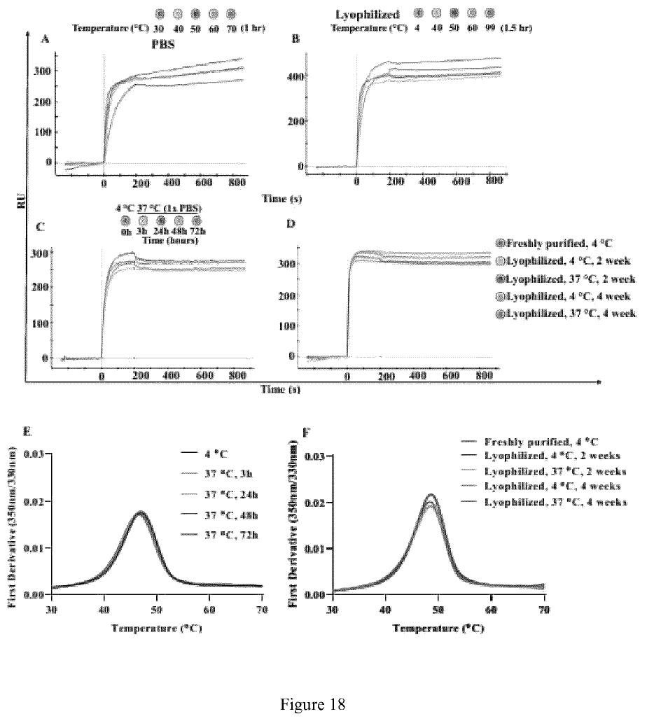

It is pertinent note that the thermal tolerance to transient and extended thermal stress is a desirable characteristic for deployment of vaccines in low resource settings in the absence of a cold-chain. Therefore, for this purpose, hCMP-mRBD protein was subjected to transient thermal stress for one hour and lyophilized hCMP-mRBD protein was subjected to transient thermal stress for ninety-minutes. Referring to FIG. 18, the hCMP-mRBD protein (SEQ ID NO: 14; vaccine candidate) in solution was observed to retain functionality after 1 hour post exposing the said protein exposure temperatures as high as 70° C. (FIG. 18A). It can also be observed from FIG. 18B, the lyophilized hCMP-mRBD also retained functionality when subjected to transient ninety-minute thermal stress upto 99° C. Further, the protein remained natively folded and at 37° C. retained functionality in solution upto three days, and for at least four weeks in the lyophilized state (FIG. 18C, FIG. 18D, FIG. 18E, FIG. 18F). In contrast, mRBD-GlyIZ protein (SEQ ID NO: 22) showed substantially decreased ACE2 binding after one-hour incubation at temperatures above 40° C. and lost ACE2 binding after lyophilization and resolubilization. (FIG. 19A, FIG. 19B).

Therefore, it can be inferred from above examples and FIGS. 15-19, that the best trimeric mRBD involved fusion with the hCMP trimerization domain at the N-terminus of mRBD. hCMP-mRBD forms a trimer that is stabilized by intermolecular disulfides and does not dissociate, even at high dilutions hCMP-mRBD shows remarkable thermotolerance. Lyophilized hCMP-mRBD was stable to extended storage at 37° C. for over four weeks and to transient 90-minute thermal stress of upto 100° C. Moreover, in contrast, to mRBD-GlyIZ (SEQ ID NO: 22), the disulfide linked hCMP-mRBD was more homogeneous and thermotolerant, thereby hCMP-mRBD (SEQ ID NO: 14) was taken forward for immunization studies in mice, guinea pigs, and hamster in the forthcoming examples.

### Example 5

### Immunization Studies

### Mice and Guinea Pig Immunizations

Group of 5, female, BALBc mice (6-8 weeks old, approximately weighing 16-18 g) and group of 5 female Hartley strain guinea pigs (6-8 weeks old, approximately weighing 300 g) were immunized with (i) 20 μg of recombinant receptor binding domain of SARS-CoV-2 (hCMP-mRBD; SEQ ID NO: 14) in 50 μl phosphate-buffered saline (pH 7.4) and mixed with AddaVax™ adjuvant (vac-adx-10)) (1:1 v/v Antigen: AddaVax™ ratio per animal/dose; (ii) 50 μl of AddaVax™) (InvivoGen, USA) adjuvant alone. Animals were immunized via the intramuscular route with two doses constituting prime and boost on Day 0 and Day 21, respectively. Sera were isolated from bleeds drawn prior to prime (day −2), post prime (day 14) and post boost (day 35).

Similar to immunization study conducted with hCMP-mRBD, immunization studies were conducted with hCMP-pRBD (Pichia expressed protein), however, AddaVax equivalent adjuvant SWE was used.

### Hamster Immunization

### Ethics and Animals' Husbandry

The animal experimental work plans were reviewed and approved by the Indian Institute of Science, Institute Animals Ethical Committee (IAEC). The experiment was performed according to CPCSEA (The Committee for the Purpose of Control and Supervision of Experiments on Animals) guidelines. The required number of Syrian golden hamsters (Mesorectums auratus) of both sex (weight 50-60 gm) were procured from the Biogen Laboratory Animal Facility (Bangalore, India). The hamsters were housed and maintained at the Central Animal Facility at IISC, Bangalore, with feed and water ad libitum. and 12 hr light and dark cycle.

### Hamster Immunization Protocol

After two-week acclimatization of animals, hamsters were randomly grouped, and the immunization protocol initiated with the pre-bleed of animals. Hamsters were immunized with 20 μg of hCMP-mRBD (SEQ ID NO: 14; subunit vaccine candidate) in 50 μl injection volume intramuscularly, with the primary on day 0 and boosts on day 21 and day 42. Bleeds were performed two weeks after each immunization.

### Virus Challenge

After completing the immunization schedule, the hamsters were transferred to the virus BSL-3 laboratory at the Centre for Infectious Disease Research, Indian Institute of Science-Bangalore (India) and were kept in individually ventilated cages (IVC), maintained at 23±1° C. and 50±5% temperature, and relative humidity, respectively. After acclimatization of seven days in IVC cages at the virus BSL-3 laboratory, the hamsters were challenged with 10⁶ PFU of SARS-Cov-2 US strain (USA-WA1/2020 obtained from BEI resources) intranasally in 100 μl of DMEM, by sedating/anaesthetizing the hamsters with a xylazine (10 mg/kg/body wt.) and ketamine (150 g/kg/body wt.) cocktail intraperitoneally. The health of hamsters, body temperatures, body weights, and clinical signs were monitored daily by an expert veterinarian. Further, based on fourteen clinical signs that were manifested in hamsters, average clinical scores were measured, which are as follows: lethargy (1 point), rough coat (1 point), sneezing (1 point), mucus discharge from nose or eyes (1 point), half closed eyes or watery eyes (1 point), huddling in the corner (1 point), ear laid back (1 point), hunched back (1 point), head tilt (1 point), moderate dyspnoea (2 points), body weight loss: 2-5% (1 point), 5-10% (2-point), 10-20% (3 point), shaking or shivering (1 point).

On the fourth day, post challenge, all the hamsters were humanely euthanized by an overdose of xylazine through intraperitoneal injection. The left lobe of the lung was harvested and fixed in 4% paraformaldehyde (PFA) for histopathological examination of lungs. The right lobes were frozen at −80° C. for determining the virus copy number by qRT-PCR.

### Histopathological Examination

Left lobes of lung, fixed in 4% of paraformaldehyde were processed, embedded in paraffin, and cut into 4 μm correct symbol, and sectioned by microtome for haematoxylin and eosin staining. The lung sections were microscopically examined and evaluated for different pathological scores by a veterinary immunologist. Four different histopathological scores were assigned as follows: Score 1: Percent of infected part of lung tissues considering the consolidation of lung; Score 2: Lung inflammation scores, considering the severity of alveolar and bronchial inflammation; Score 3: Immune cell influx score, considering the infiltration of lung tissue with the numbers of neutrophils, macrophages and lymphocytes; Score 4: edema score, considering the alveolar and perivascular edema. The scores and parameters were graded as absent (Score 0), minimal (Score 1), mild (Score 2), moderate (Score 3), or severe (Score 4).

RNA Extractions and q-RT-PCR to Quantitate Sub Genomic Viral RNA in Lungs

Three-time freeze-thawed right lower lobe from the lung of each hamster was homogenized in 1 ml of RNAiso Plus Reagent (Takara) and total RNA was isolated as per the manufacturer's protocol using chloroform and isopropanol reagents. The quantity and quality (260/280 ratios) of RNA extracted was measured by Nanodrop. The extracted RNA was further diluted to 27 ng/μl in nuclease free water. The viral sub genomic RNA copy number was quantified by using 100 ng of RNA/well for 10 μl of reaction mixture using AgPath-ID™ One-Step RT-PCR kit (AM1005, Applied Biosystems). The following primers and probes were used 2019-nCoV_N1-Fwd-5′GACCCCAAAATCAGCGAAAT3′ (SEQ ID NO: 99); 2019-nCoV_N1-Rev 5′TCTGGTTACTGCCAGTTGAATCTG3′ (SEQ ID NO: 100); 2019-nCoV_N1 Probe (6-FAM/BHQ-1) ACCCCGCATTACGTTTGGTGGACC (Sigma Aldrich) (SEQ ID NO: 101) for amplifying RNA from the SARS CoV-2 N-1 gene. The sub genomic virus copy number per 100 ng of RNA was estimated by generating a standard curve from a known number of pfu of the virus.

### ELISA-Serum Binding Antibody End Point Titers

Desired vaccine antigens (hCMP-mRBD; SEQ ID NO: 14) 4 μg/mL, in 1×PBS, 50 μL/well) were coated on 96 well plates for two hours and incubated on a MixMate thermomixer (Eppendorf, USA) at 25° C. under constant shaking (300 rpm). Antigen immobilization was assessed by coating ACE2-hFc protein, as a control. Subsequently, coated wells were washed with PBST (200 μl/well) four times, and blocked using blocking solution (100 μL, 3% skimmed milk in 1×PBST) and then incubated at 25° C. for one hour, 300 rpm. Post blocking, antisera were diluted four-folds serially, starting 1:100 and incubated at 25° C. for 1 hour, 300 rpm. Post sera binding, three washes were performed (200 μL of 1×PBST/well). Following this, anti-Guinea Pig IgG secondary antibody (ALP conjugated, Rabbit origin) (diluted 1:5000 in blocking buffer) (50 μL/well) was added and incubated at 25° C. for 1 hour, 300 rpm (Sigma-Aldrich). Post incubation, four washes were performed (200 μL of 1×PBST/well) and incubated with pNPP liquid substrate (50 μL/well) (pNPP, Sigma-Aldrich) at 37° C. for 30 minutes, 300 rpm. Finally, the chromogenic signal was measured at 405 nm. The highest serum dilution possessing signal above cutoff (0.2 O.D. at 405 nm) was considered as the endpoint titer for ELISA.

### Convalescent Patient Sera Samples

Convalescent patient sera were drawn (n=40) and assayed for pseudoviral neutralization as described in the following pseudovirus neutralization section. The ethics approval of human clinical samples were approved by Institute Human Ethical Committee.

### Production of Pseudo-Type SARS-CoV-2 and Pseudovirus Neutralization Assay

Pseudovirus neutralization assays were performed with SARS-CoV-2 pseudo virus harbouring reporter NanoLuc luciferase gene. Briefly, HEK293T cells were transiently transfected with plasmid DNA pHIV-1 NL4.3Aenv-Luc and Spike-A19-D614G by using ProFection™ mammalian transfection kit (Promega Inc) following the instructions in the kit manual. Post 48 hours, the pseudovirus containing culture supernatant was centrifuged for 10 mins at 600×g followed by filtration via 0.22 μm filters, and stored at −80° C. until further use. 293T-hACE-2 (BEI resources, NIH, Catalog No. NR-52511) or Vero/TMPRSS2 (JCRB cell bank, JCRB #1818) cells expressing the ACE2 or ACE and TMPRSS2 receptors respectively were cultured in DMEM (Gibco) supplemented with 5% FBS (Fetal Bovine Serum), penicillin-streptomycin (100 U/mL). Patient derived convalescent sera (n=40) were tested for neutralization in both 293T-ACE-2 and Vero/TMPRSS2 cells, whereas animal sera were tested only in Vero/TMPRSS2 cells. Neutralization assays were done in two replicates by using heat-inactivated animal serum or human COVID-19 convalescent serum (HCS). The pseudovirus (PV) was incubated with serially diluted sera in a total volume of 100 μL for 1 hour at 37° C. The cells (Vero/TMPRSS2 or 293T-hACE2) were then trypsinised and 1×10⁴ cells/well were added to make up the final volume of 200 uL/well. The plates were further incubated for 48 hours in humidified incubator at 37° C. with 5% CO₂. After 48 hours of incubation, 140 μL supernatant was removed and 50 μL Bright-Glo luciferase substrate (Promega Inc.) was added. After 2-3 minutes incubation, 80 μL lysate was transferred to white plates and luminescence was measured by using Cytation-5 multi-mode reader (BioTech Inc.) The luciferase activity measured as Relative luminescence units (RLU) from SARS-CoV-2 pseudovirus in the absence of sera was used as reference for normalizing the RLUs of wells containing sera. Pseudovirus neutralization titers (ID₅₀) were determined as the serum dilution at which infectivity was blocked by 50%. The three RBD mutations were introduced into the parental clone using overlap PCR and Gibson recombination.

### Statistical Analysis

The P values for ELISA binding titers, neutralization titers, were analysed with a two-tailed Mann-Whitney test using the GraphPad Prism software. The P values for pairwise Wt and SA pseudovirus neutralization titers were analysed utilizing the Wilcoxon Rank-Sum test. The P value for weight change between virus control and unchallenged groups was analysed by two-tailed student-t test. The correlation coefficients for pseudovirus neutralization 293T-ACE2/VeroE6-TMPRSS2 cell line pseudovirus neutralizations were analysed by Spearman correlation using the GraphPad Prism software.

### Results

Trimeric mRBD Elicits High Titers of Neutralizing Antibodies in Mice and Guinea Pigs and Protects Hamsters from Viral Challenge

The monomeric mRBD derivatives and trimeric mRBD derivatives having trimerization domain (like hCMP) were adjuvanted with SWE, an AddaVax™ and MF59 equivalent adjuvant, in BALB/c mice. Animals were immunized intramuscularly at day 0 regimen, followed by a boost at day 21. Two weeks post boost, sera were assayed for binding and neutralizing antibodies.

### (a) ELISA Assay and Neutralization Assay: Mice

Table 9 summarizes the results of ELISA assay showing binding titer values against the antigens RBD2 and Spike-2P protein in the sera of mice immunized with various vaccine agent (candidates) adjuvanted with AddaVax™. The sera of mice was further tested for competition with ACE-2-Fc to check the whether the antibodies generated in mice immunized with various vaccine agents of the present disclosure compete in the presence of ACE2 for binding to spike antigen on the ELISA plate. Further, it was tested if the serum neutralizes the live SARS-CoV-2 virus. The results of ACE2-Fc competition serology assay, and the neutralization assay are also provided in Table 9

TABLE 9
Mice immunized with Adda Vax ™

| | | | | | GM- |
|---|---|---|---|---|---|
| | | GM- | GM- | | Neutral- |
| | | ELISA | ELISA | GM-ACE-2 | ization |
| S. | Vaccine | Titer on | Titer on | Competition | Titer |
| NO. | Agent | RBD2 | Spike-2P | Titer (IC50) | ID50 |
| 1. | mInCV01R | 409599 | 289630 | 163.78 | 2469.43 |
| | (SARS CoV- | | | | |
| | 2 RBD); SEQ | | | | |
| | ID NO: 8 | | | | |
| 2. | mInCV02R | 1212.57 | 1600 | 2 | 100 |
| | (SARS CoV- | | | | |
| | 2 RBD N-1); | | | | |
| | SEQ ID | | | | |
| | NO:10 | | | | |
| 3. | iInCV01R | 102399.9 | 33779.4 | 4.79 | 138 |
| | (SARS CoV- | | | | |
| | 2 RBD); SEQ | | | | |
| | ID NO: 56 | | | | |
| 4. | iInCV02R | 135117.6 | 44572.18 | 7.7 | 509.24 |
| | (SARS CoV- | | | | |
| | 2 RBD); SEQ | | | | |
| | ID NO: 58 | | | | |
| 5. | mInCV21R | 178288.7 | 540470.4 | 1151.3 | 17393.5 |
| | (SARS CoV- | | | | |
| | 2 hCMP- | | | | |
| | RBD); SEQ | | | | |
| | ID NO: 14 | | | | |
| 6. | Mutant | 58813.3 | 44572.18 | 510.18 | 6248.2 |
| | variant M15 | | | | |
| 7. | Mutant | 102399.9 | 135117.6 | 346.34 | 13950.71 |
| | variant M21 | | | | |
| 8. | Protein | 310418.75 | 178288.75 | 1141.17 | 48572.96 |
| | DM37; SEQ | | | | |
| | ID NO: 69 | | | | |

Referring to Table 9, it can be inferred that the endpoint titers to RBD2 antigen, and spike-2P antigen measured in the mice sera after the boost, ranged between 1:1212.57 to 1:409599 and 1:1600 to 540470.4, respectively.

High ELISA titers are correlated with high neutralization titers. For reference, the GMT neutralization ID50 in human convalescent sera (HCS) is about 125, when measured in same neutralization assay. Hence, the fold increase over the HCS ID50 is a measure of the immunogenicity of the formulation. For comparison the Astra Zeneca and Bharat Biotech vaccines have a ratio close to 1.

Table 10 summarizes the results of ELISA assay showing binding titer values against the antigens RBD2 and Spike-2P protein in the sera of mice immunized with various vaccine agents (candidates) adjuvanted with SWE.

TABLE 10
Mice immunized with SWE

| | | | | GM- | GM- |
|---|---|---|---|---|---|
| | | GM- | GM-ELISA | ACE-2 | Neutral- |
| | | ELISA | Titer | Compt | ization |
| S. | | Titer on | on | Titer | Titer |
| NO. | Vaccine Agent | RBD2 | Spike-2P | (IC50) | ID50 |
| 1. | mInCV01R | 19401.17 | 4850.29 | 7.9 | 94.86 |
| | (SARS CoV-2 | | | | |
| | RBD); SEQ ID | | | | |
| | NO: 8 | | | | |
| 2. | mInCV02R | 6400 | 11143.04 | 26.87 | 707.64 |
| | (SARS CoV-2 | | | | |
| | RBD N-1); SEQ | | | | |
| | ID NO:10 | | | | |
| 3. | mInCV21R | 102399.9 | 135117.61 | 1899.73 | 31706.39 |
| | (SARS CoV-2 | | | | |
| | hCMP-RBD); | | | | |
| | SEQ ID NO:14 | | | | |
| 4. | mInCV26R | 135117 | 102400 | 575.44 | 7471.78 |
| | (SARS CoV-2 | | | | |
| | RBD with | | | | |
| | hCMP at C- | | | | |
| | terminal); SEQ | | | | |
| | ID NO: 16 | | | | |
| 5. | mInCV29R | 178288.75 | 58813.3 | 1119.93 | 12505 |
| | (SARS CoV-2 | | | | |
| | RBD with | | | | |
| | GlyIZ at C- | | | | |
| | terminal); SEQ | | | | |
| | ID NO: 22 | | | | |
| 6. | Mutant variant | 135117.61 | 178288.75 | 1259.54 | 14518.48 |
| | M15 | | | | |
| 7. | Mutant variant | 713155.02 | 25599.99 | 96.99 | 7365.63 |
| | M21 | | | | |
| 8. | pDM48R | 8444.85 | 19401.17 | 10.64 | 1527.4 |
| | mutant variant; | | | | |
| | SEQ ID NO: 72 | | | | |
| 9. | pDM49R | 77604.68 | 77604.68 | 1118.33 | 11366.77 |
| | mutant variant; | | | | |
| | SEQ ID NO: 73 | | | | |
| 10. | DM37-SA | 102400 | 33779.4 | ND | ND |
| | (South African) | | | | |
| | mutant variant; | | | | |
| | SEQ ID NO: 79 | | | | |
| 11. | hCMP-DM37 | 33779.4 | 102400 | ND | ND |
| | mutant variant; | | | | |
| | SEQ ID NO: 81 | | | | |
| 12. | hCMP- | 36203.86 | 51200 | ND | ND |
| | DM37SA | | | | |
| | mutant variant; | | | | |
| | SEQ ID NO: 83 | | | | |
| 13 | DM-37a | 8444.8 | 33779.4 | ND | ND |
| | mutant variant; | | | | |
| | SEQ ID NO: 77 | | | | |
| 14. | DM37 mutant | 102400 | 44572.18 | ND | ND |
| | variant + | | | | |
| | DM37-SA | | | | |
| | mutant variant; | | | | |
| | SEQ ID NO: 69 + | | | | |
| | SEQ ID NO: | | | | |
| | 79 | | | | |
| 15. | hCMP-DM37 | 178288.75 | 77604.68 | ND | ND |
| | mutant variant + | | | | |
| | hCMP- | | | | |
| | DM37SA; SEQ | | | | |
| | ID NO: 81 + | | | | |
| | SEQ ID NO: 83 | | | | |

ND: Not determined

The ELISA titer and neutralization titer values of the vaccine candidate as shown in Table 10 shows that the vaccine candidates have high immunogenicity to elicit an enhanced immune response.

High ELISA titers are correlated with high neutralization titers. For reference, the GMT neutralization ID50 in human convalescent sera (HCS) is about 125, when measured in same neutralization assay. Hence, the fold increase over the HCS ID50 is a measure of the immunogenicity of the immunogenic composition (vaccine formulation).

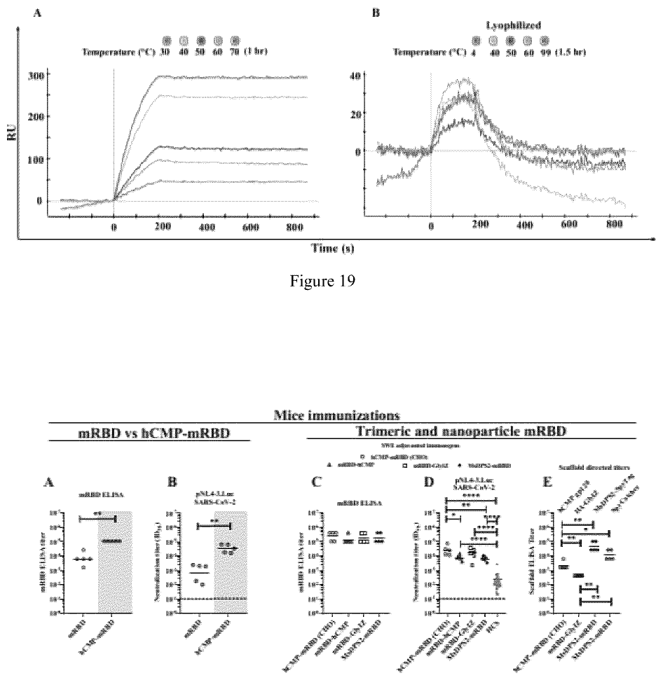

Referring to FIG. 20A, trimeric hCMP-mRBD (SEQ ID NO: 14) adjuvanted with SWE, elicited 16-fold higher mRBD binding titers as compared to monomeric mRBD. It can be observed from FIG. 20B that pseudoviral neutralization titers elicited by trimeric hCMP-mRBD were 45-fold higher (hCMP-mRBD GMT: 31706, mRBD GMT: 707, P=0.008) as compared to monomeric mRBD.

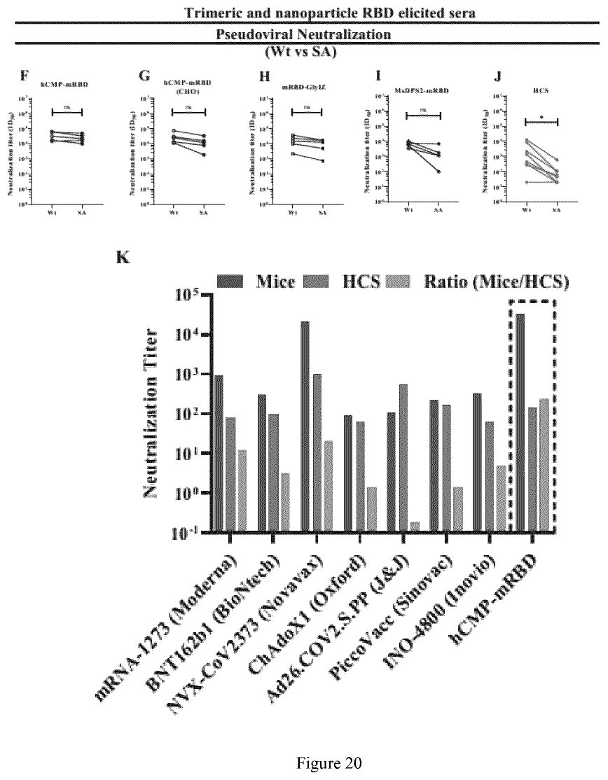

Further, the immunogenicity of hCMP-mRBD adjuvanted with AddaVax™ and SWE respectively, was compared. The mRBD binding titers and pseudoviral neutralization titers were similar in both adjuvants, confirming their functional equivalence (FIG. 21A, FIG. 21B).

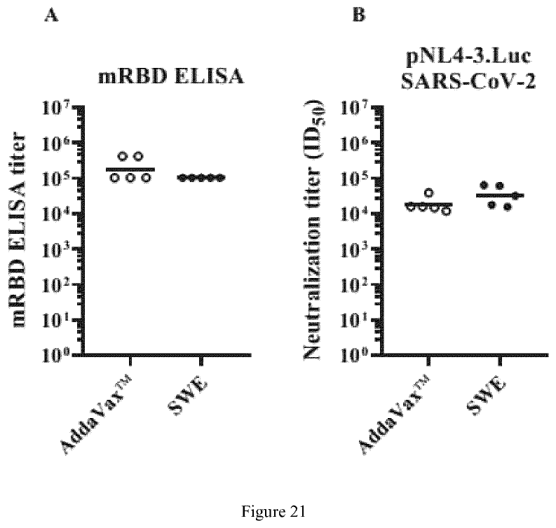

Next, the immunogenicity of trimeric, SWE adjuvanted hCMP-RBD derived from different expression platforms, namely CHO and Pichia stable cell lines was assessed. The binding titers were 12-fold higher in CHO-derived hCMP-mRBD compared to hCMP-pRBD (p=0.008) (FIG. 21C, FIG. 22). CHO-derived hCMP-mRBD (GMT: 24086) elicited high pseudoviral neutralizing titers as compared to sera elicited by Pichia expressed hCMP-pRBD which showed negligible neutralization (P=0.008) (FIG. 21D, FIG. 22).

Referring to FIG. 21C, it can be observed that N-terminal trimerized mRBD derived from CHO cells (hCMP-mRBD GMT: 235253) elicited similar mRBD binding titers compared to C-terminal trimerized mRBD-hCMP (SEQ ID NO: 16) in SWE adjuvanted formulations. Moreover, it can also be observed from FIG. 21D that the pseudoviral neutralization titer elicited by N-terminal trimerized hCMP-mRBD (SEQ ID NO: 14) (GMT: 24086) was 3-fold (P=0.0317) and 2-fold (P=0.42) higher compared to C-terminal trimerized mRBD-hCMP (SEQ ID NO: 16) (GMT: 7472) and mRBD-GlyIZ (SEQ ID NO: 22) (GMT: 12505), respectively. MsDPS2-mRBD nanoparticle adjuvanted with SWE, elicited similar mRBD binding antibody titers compared to hCMP-mRBD (GMT: 235253) (FIG. 21C) but approximately 4-fold (P=0.008) lower pseudoviral neutralization titers (GMT: 24086) as compared to MsDPS2-mRBD (GMT: 6181) (FIG. 21D).

The hCMP trimerization domain and nanoparticle scaffolds also elicited binding antibodies. The binding titers directed towards the glycosylated IZ were measured by ELISA utilizing influenza HA stem fused to GlyIZ as the immobilized antigen and it can be observed from FIG. 21E that binding titers of mRBD-GlyIZ were the lowest (GMT: 400), and 5-fold lower compared to the binding titers of hCMP-mRBD (GMT: 2111). The binding titers of hCMP-mRBD were estimated using hCMP V1cyc JRFL gp120 containing the same trimerization domain. It can also be observed from FIG. 21E that the MsDPS2-SpyTag and SpyCatcher titers were 111-fold (GMT: 44572, P=0.0079), and 28-fold higher (GMT: 11143, P=0.0079) as compared to GlyIZ directed titers, respectively.

Pseudoviral Neutralization Titers Against Wildtype and Pseudovirus with South African (B.1.351, SA) RBD Mutations

The ability of the anti-sera to neutralize pseudovirus containing the RBD mutations present in the South African isolate (B.1.351, SA) (K417N, E484K and N501Y) was measured. Referring to FIG. 21F, FIG. 21G, FIG. 21H, it can be observed that sera elicited by hCMP-mRBD, mRBD-hCMP and mRBD-GlyIZ neutralized South African (SA) pseudovirus with 1.4-2.4 fold lower titers as compared to wild type pseudovirus (P=0.05-0.06) Further, nanoparticle MsDPS2-mRBD elicited sera neutralized the SA virus with 5.6-fold lower titers compared to wild type pseudovirus (FIG. 21 I). Referring to FIG. 21J, it can be observed that high titer Human convalescent sera (HCS) neutralized the SA virus with 14-fold lower titer (SA GMT: 59) compared to wild type (GMT: 845), and hence it can be concluded the RBD formulations of the present disclosure (hCMP-mRBD, hCMP-mRBD (CHO), mRBD-hCMP, and mRBD-GlyIZ showed only a small (approximately 2-3 fold) decrease in neutralization titers with the pseudovirus containing the three South African RBD mutations (K417, E484K, N501Y) in contrast to human convalescent sera (HCS) that failed to neutralize the SA virus.

### Guinea Pig Immunizations

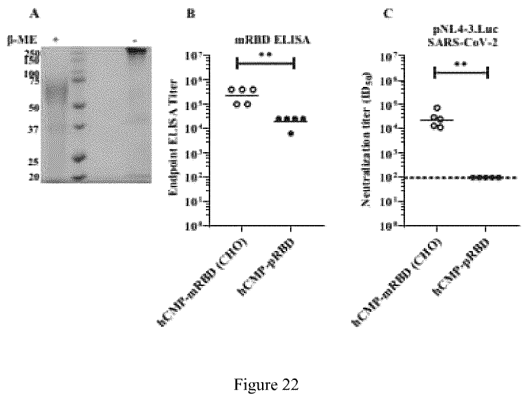

The immunogenicity of hCMP-mRBD (SEQ ID NO: 14) adjuvanted with AddaVax™ in guinea pig immunizations following prime (Week 0) and, two boosts (week 3 and week 6), was assessed. Referring to FIG. 23A and FIG. 23B, both binding and neutralization titers were significantly enhanced following the second boost. Trimerization scaffold directed titers in guinea pigs showed only a marginal increase after the second boost (FIG. 23C). Sera collected after the second boost neutralized SA pseudovirus (GMT: 8252) with 4.3-fold lower titer compared to wildtype (GMT: 35693), while corresponding sera from spike-2P immunized animals showed a 15-fold drop (FIG. 23D, and FIG. 23E). However, sera after the first boost were not available to assay against the SA pseudovirus. Therefore, it can be inferred from FIG. 23 that both mice and guinea pigs did not elicit any binding antibodies to the L14 linker present in the immunogens.

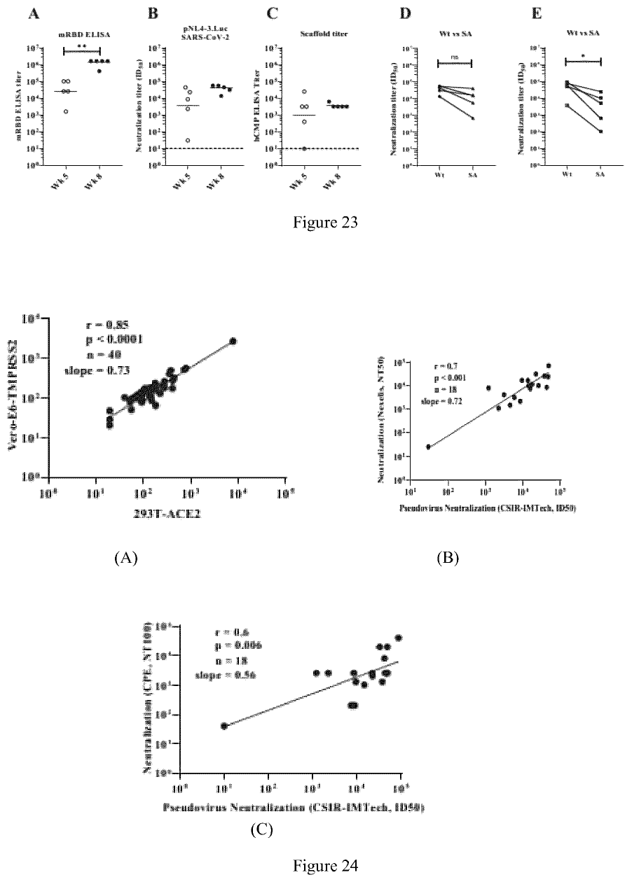

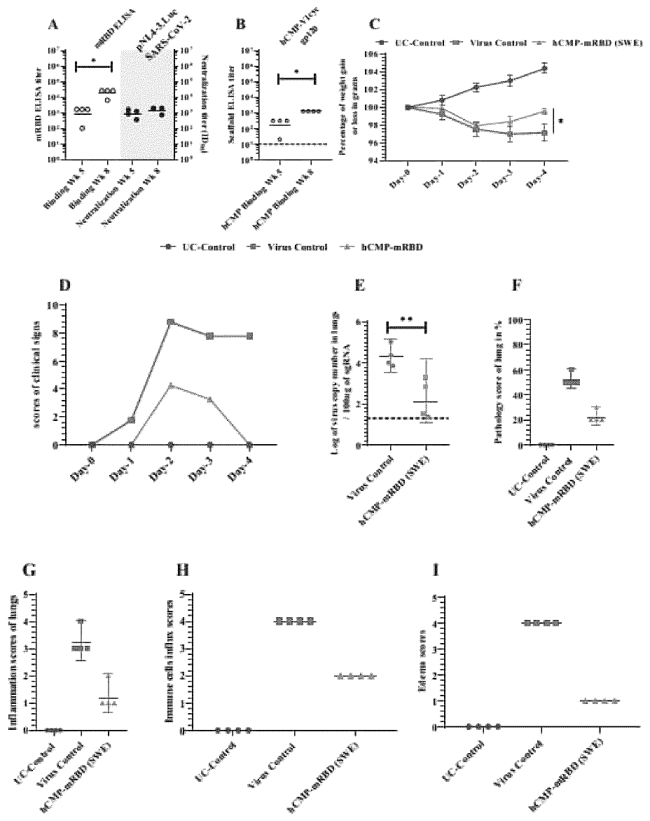

Further, from the pseudoviral neutralization titer correlation as depicted in FIG. 24, it can be observed that pseudoviral neutralization titers correlated well in two independent assay platforms performed with an identical set of sera and with live virus neutralization titers from a CPE based assay. Additionally, from a dose sparing study involving 5 μg of hCMP-mRBD adjuvanted with SWE, it can be concluded that the mRBD binding titers were observed to be marginally higher as compared to the 20 μg dose (FIG. 25A), and similar results were observed with pseudoviral neutralization titer were similar. (FIG. 25B).

### Immunogenicity Comparisons

The immunogenicity of trimeric hCMP-mRBD (SEQ ID NO: 14) was compared with many approved vaccine formulations as shown in FIG. 21K. It can be observed from FIG. 21K that trimeric hCMP-mRBD elicited exceedingly high neutralizing antibodies in mice, as compared to human Convalescent Sera (HCS) titers assayed in the identical assay platform. Additionally, both the mice neutralizing antibody titers and their ratio relative to HCS neutralizing titers, compared favourably with corresponding values for vaccine candidates being tested in the clinic or provided with emergency use authorizations. It is known from the previous studies that for COVID-19 vaccines, mice titers are predictive of those in humans. Therefore, trimeric hCMP-mRBD (SEQ ID NO: 14) is a potential vaccine candidate for COVID-19.

### Hamster Immunizations

Further, to examine the efficacy of hCMP-mRBD, hamster immunization and challenge study was conducted. Hamsters were immunized with hCMP-mRBD at week 0, 3 and 6. Two weeks post boost, sera were assayed for binding and neutralizing antibodies.

### (a) ELISA Assay and Neutralization Assay: Mice

Table 11 summarizes the results of ELISA assay showing binding titer values against the antigens RBD2 and Spike-2P protein in the sera of mice immunized with various vaccine agent (candidates) adjuvanted with AddaVax™. The sera of mice was further tested for competition with ACE-2-Fc to check the whether the antibodies generated in mice immunized with various vaccine agents of the present disclosure compete in the presence of ACE2 for binding to spike antigen on the ELISA plate. Further, it was tested if the serum neutralizes the live SARS-CoV-2 virus. The results of ACE2-Fc competition serology assay, and the neutralization assay are also provided in Table 11

TABLE 11
Hamster immunized with AddaVaxTM

| | | | | GM- | GM- |
|---|---|---|---|---|---|
| | | GM- | | ACE-2 | Neutral- |
| | | ELISA | GM-ELISA | Compt | ization |
| S. | | Titer on | Titer on | Titer | Titer |
| NO. | Vaccine Agent | RBD2 | Spike-2P | (IC50) | ID50 |
| 1. | iInCV01R | 36203.8 | 1600 | 100 | 741.18 |
| | (SARS CoV-2 | | | | |
| | RBD); SEQ ID | | | | |
| | NO: 56 | | | | |
| 2. | mInCV21R | 1007.93 | 800 | 23.74 | ND |
| | (SARS CoV-2 | | | | |
| | hCMP-RBD); | | | | |
| | SEQ ID NO: 14 | | | | |

ND: Not determined

As shown in Table 11, the ELISA and titer values of vaccine candidate (SEQ ID NO: 56) indicates that it can act as suitable candidate for eliciting an enhanced immune response in a subject.

Table 12 summarizes the results of ELISA assay showing binding titer values against the antigens RBD2 and Spike-2P protein in the sera of mice immunized with various vaccine agents (candidates) adjuvanted with SWE.

TABLE 12
Hamster immunized with SWE

| | | | | GM- | GM- |
|---|---|---|---|---|---|
| | | GM- | GM-ELISA | ACE-2 | Neutral- |
| | | ELISA | Titer | Compt | ization |
| S. | | Titer on | on | Titer | Titer |
| NO. | Vaccine Agent | RBD2 | Spike-2P | (IC50) | ID50 |
| 1. | mInCV21R | 800 | 800 | 21.73 | 534.53 |
| | (SARS CoV-2 | | | | |
| | hCMP-RBD); | | | | |
| | SEQ ID NO: 14 | | | | |

Referring to Table 12, it can be inferred that High ELISA titers are correlated with high neutralization titers of vaccine candidate (SEQ ID NO: 14). The fold increase over the HCS ID50 is a measure of the immunogenicity of the immunogenic composition (vaccine formulation). Therefore, the high ELISA titers and high neutralization titers indicates that vaccine candidate (SEQ ID NO: 14) elicits an enhanced immune response.

As shown in FIG. 25A, the mRBD binding titers (GMT:18101) and neutralization titers (GMT: 1423) were lower than those observed in guinea pigs and mice, wherein neutralization titers remained unchanged between the first and second boost. Further, the scaffold directed titers were approximately 103, consistent with the low sequence identity of hCMP (51%) with the hamster ortholog, as depicted in FIG. 25B. Following immunization, animals were challenged with replicative Wildtype virus. Two additional groups, namely unimmunized-unchallenged (UC) and unimmunized-virus challenged (VC) animals, acted as controls. Post infection, the immunized animals regained weight and showed markedly lower clinical signs (FIG. 25C, and FIG. 25D). The lung viral titers and histopathology scores were relative to the VC control group (FIG. 25F to FIG. 25J). The tissue sections as depicted in FIG. 25J showed clear lung epithelial interstitial spaces and minimal immune cell infiltration in the immunized group as compared to virus challenged group.

Hence, all animals remained healthy after the immunizations with hCMP-mRBD (SEQ ID NO: 14). Therefore, it can be concluded that hCMP mediated trimerization of mRBD led to elicitation of robust binding and neutralizing antibodies considerably in excess of those seen in human convalescent sera, that protected hamsters from high dose, replicative viral challenge.

Characterization of hCMP-mRBD Expressed from Permanent Cell Lines

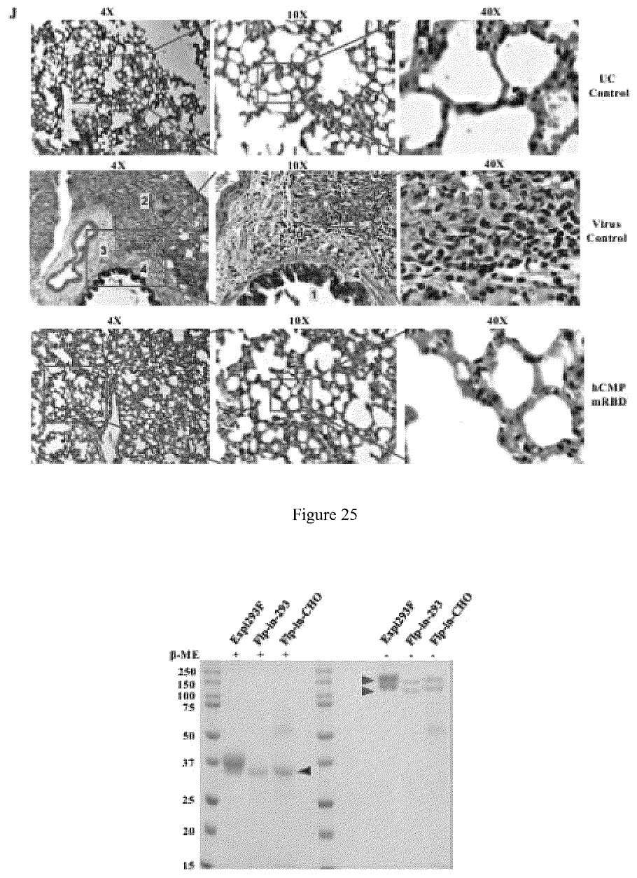

Stable Chinese hamster ovary (CHO) and HEK293 suspension cell lines expressing the protein (hCMP-mRBD) were constructed. Purified protein yields were 80-100 mg/liter, similar to those expressed in Expi293 cells, and SDS-PAGE revealed the presence of disulfide linked trimers (FIG. 26). CHO expressed protein (hCMP-mRBD) adjuvanted with SWE adjuvant has comparable immunogenicity in mice to transiently expressed protein, as depicted in FIG. 21A-FIG. 21D.

hCMP-pRBD protein was also expressed in the methylotrophic yeast Pichia. pastoris at a purified yield of approximately 7 mg/liter. As observed from FIG. 22, FIG. 15D, and FIG. 26, the hCMP-pRBD protein was more heterogeneous and formed high molecular weight aggregates unlike mammalian cell expressed proteins. In mice, formulations with the AddaVax equivalent adjuvant SWE elicited low mRBD and negligible neutralization titers after two immunizations (FIG. 22B, FIG. 22C).

Overall, it can be inferred that the oligomeric RBD formulations (hCMP-pRBD, hCMP-mRBD (CHO) (SEQ ID NO: 81), mRBD-hCMP (SEQ ID NO: 16), and mRBD-GlyIZ; (SEQ ID NO: 22) are highly immunogenic and thermotolerant. Neutralization titers in small animals were 20-300 folds higher than in convalescent sera, showing much better immunogenicity then virtually all currently licensed vaccines when compared in the same animal model (mouse). Mouse sera showed potent neutralization against pseudovirus containing the B.1.351 SA RBD mutations with only a small, i.e., approximately three-fold drop in neutralization titer, in contrast to virtually complete loss of neutralization seen in most convalescent sera.

### Example 6

### Stability Profile of Mutant Variants

The present example describes the thermal stability of vaccine candidates (for instance, mutant variants). For the purpose of measuring the thermal stability of the vaccine candidates as described herein, wild type RBD: RBD1 (SEQ ID NO: 2); RBD 2 (SEQ ID NO: 4); RBD3 (SEQ ID NO: 6) and its mutants expressed in mammalian cells and dialyzed in 1×PBS, were subjected to thermal denaturation on nano-DSF (Prometheus NT.48). The wild type (WT) RBD as described herein, was always kept as a control during thermal denaturation and the protein concentration was kept between 0.1 mg/ml to 0.3 mg/ml.

Table 13 shows the delta-Tₘ values indicating the stability profile of the mutant variants as potential vaccine candidates.

TABLE 13

| S. NO. | Vaccine Agent | Delta-Tm value |
|---|---|---|
| 1. | M1 | 0.3 |
| 2. | M3 | 2.2 |
| 3. | M4 | 1.4 |
| 4. | M5 | 1.3 |
| 5. | M6 | 0.5 |
| 6. | M9 | 1.8 |
| 7. | M11 | 0.9 |
| 8. | M12 | 0.4 |
| 9. | M14 | 0.7 |
| 10. | M15 | 3.3 |
| 11. | M17 | 1.3 |
| 12. | M18 | 3.4 |
| 13. | M20 | 3.8 |
| 14. | M21 | 4.4 |
| 15. | M22 | 4 |
| 16. | M24 | 5 |
| 17. | M25 | 3.5 |
| 18. | M26 | 7 |
| 19. | M27 | 6.3 |
| 20. | M28 | 6 |
| 21 | DM2 | 1.6 |
| 22. | DM8 | 4.4 |
| 23. | DM9 | 1.3 |
| 24. | DM10 | 1.6 |
| 25. | DM11 | 1.6 |
| 26 | DM21 | 2.7 |
| 27. | DM24 | 0.2 |
| 28. | DM26 | 4.3 |
| 29. | DM35 | 2.6 |
| 30. | DM36 | 8 |
| 31. | DM37; SEQ ID NO: 69 | 7 |
| 32. | DM38 | 7.6 |
| 33. | DM39 | 7.6 |
| 34. | DM40 | 7.7 |
| 35. | DM42 | 4.6 |
| 36. | DM43 | 1.3 |
| 37. | DM46; SEQ ID NO: 85 | 4.4 |
| 38. | DM47 | 3.6 |
| 39. | DM48 | 1.4 |
| 37. | BLM4 | 0.9 |
| 38. | BLM5 | 0.3 |
| 39. | BLM6 | 1.9 |
| 40. | BLM7 | 1.4 |
| 41. | BLM8 | 1 |
| 42. | BLM9 | 1 |
| 43. | BLM10 | 2.2 |
| 44. | BLM11 | 1.5 |
| 45. | BLM12 | 2.3 |
| 46. | BLM14 | 0.9 |
| 45. | BLM15 | 0.8 |
| 46. | BLM16 | 2.1 |
| 47. | BLM18 | 3.1 |
| 48. | BLM19 | 2.7 |
| 49. | BLM20 | 0.4 |
| 50. | BLM22 | 0.6 |
| 51. | pDM49R; SEQ ID NO: 73 | 4.8 |
| 52. | pDM49 + SA MUTATION; | 1.4 |
| | SEQ ID NO: 74 | |
| 53. | BMM1 | 6.5 |
| 54. | BMM2 | 7 |
| 55. | BMM5 | 4.8 |
| 56. | BMM6 | 7.7 |
| 57. | BMM7 | 7.1 |
| 58. | BMM8 | 7.8 |
| 59. | BMM9 | 6.1 |
| 60. | BMM11 | 7.7 |

As shown in Table 13, the mutants or vaccine candidate having delta Tm (mutant(tm)-WT(tm)) values higher than zero were considered as stabilized mutants.

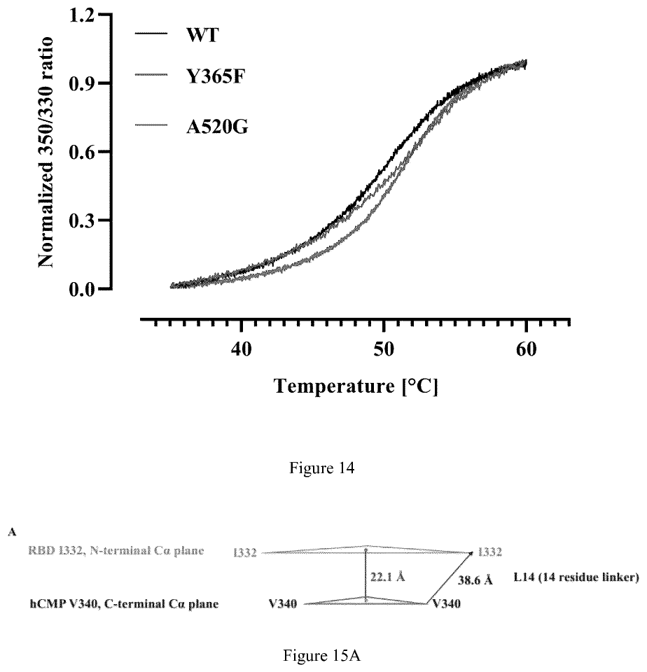

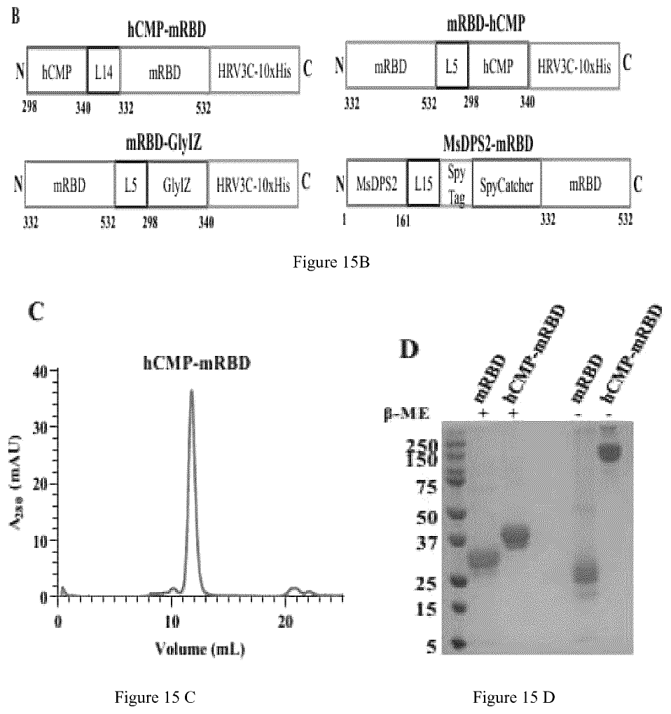

The present disclosure also identified two mutations that are possible in the vaccine candidates as described herein. The variants are Y365F and A520G which were identified by yeast two hybrid system in SARS-CoV-2 RBD (331-532) (SEQ ID NO: 2) (FIG. 13). The mutants were also found to be thermally stable (FIG. 14). It is contemplated that such variants can provide the desired results when performed with other proteins (vaccine candidates) as disclosed herein. The vaccine candidates as disclosed in the present disclosure are referred to as immunogenic composition which further comprises pharmaceutically acceptable carriers, wherein the pharmaceutically acceptable carriers are selected from the group consisting of adjuvants and excipients. The adjuvants and excipients that are known to a person skilled in the art can be added to the immunogenic composition (vaccine). In an example, the pharmaceutically acceptable carriers are selected from the group consisting of Alhydrogel (aluminium hydroxide adjuvant), Alhydrogel CpG, Addavax (oil-in-water adjuvant), SWE (squalene-in-water emulsion adjuvant), and MF59.

The present disclosure also discloses an immunogenic composition (vaccine) comprising a combination of two polypeptide. The presence of the combination of two polypeptides makes the immunogenic composition more thermostable. Also, when such an immunogenic composition is administered in a subject elicits an enhanced immune response in a subject. This can be inferred from Table 10, wherein the vaccine candidates: (i) DM37 mutant variant+DM37-SA mutant variant; SEQ ID NO: 69+SEQ ID NO: 79; and (ii) hCMP-DM37 mutant variant+hCMP-DM37SA; SEQ ID NO: 81+SEQ ID NO: 83 elicits high ELISA titer of neutralizing antibodies.

Their increased thermostability confers advantages for vaccine production, formulation, and storage without requiring continuous refrigeration (cold-chain), that would help in combating COVID-19.

### Example 7

### Comparative Example

In this example, the immunogenicity of the subunit vaccines candidates of the present disclosure were compared with that of the mammalian expressed full length RBD region (mFLR) (SEQ ID NO: 86; 327-537) to evaluate the effectivity of the vaccine formulation of the present disclosure. Table 14 shows the immunization ELISA titer values of full length RBD region (SEQ ID NO: 86; 327-537) and subunit vaccine candidate of the present disclosure, in mice.

TABLE 14
Mice immunized with SWE

| | | GM-ELISA Titer | GM-ELISA Titer |
|---|---|---|---|
| S. NO. | Vaccine Agent | on RBD2 | on Spike-2P |
| 1. | Full length RBD region | 2111.21 | 2785.76 |
| | (mFLR); SEQ ID NO: 86 | | |
| | (Control) | | |
| 2. | mRBD2-E484K | 11143 | 6400 |
| 3. | DM21 | 19401.17 | 25600 |
| 4. | DM26 | 25600 | 25600 |

The immunization ELISA titers in mice (as shown in Table 14) shows that mFLR has significantly lower titers than subunit vaccines candidates of the present disclosure, all with a single amino acid substitution: mRBD2-E484K, DM21 and DM26.

Further, it was also observed that the expression levels of mFLR was lower (80-100 mg/L), as compared to RBD2 (SEQ ID NO: 4), which is 200 mg/L. Overall, it was observed that RBD1 (SEQ ID NO: 2) and RBD2 (SEQ ID NO: 4), and its variants, showed higher immunization titers as compared to mFLR. Therefore, it can be inferred that the immunogenic composition (subunit vaccine candidates) of the present disclosure are more stable and elicits an enhanced immune response when immunized in a subject, as compared to that of mFLR, wherein mFLR exhibits showed poor characteristics in stability, immunogenicity, etc.

### Advantages of the Present Disclosure

The present disclosure discloses the first generational subunit-based vaccine candidate for SARS-CoV-2 that can be mass-produced across the globe to cater to the need of the hour. The present disclosure discloses three different constructs with addition or deletion of N-terminal glycosylation site leading to nCV01R (RBD1; 331-532) and nCV02R (RBD2; 332-532) versions, and third version with deletion of N and C-terminal glycosylation sites leading to nCV22R (RBD3; 332-530). The construct with RBD1 (SEQ ID NO: 2; 331-532) has the advantage of high yielding vaccine candidate protein, whereas the construct with RBD2 (SEQ ID NO: 4; 332-532) has the advantage of conferring properties like high immunogenicity in a subject. The present disclosure is the first of its study to describe the glycan engineered version of the RBD and has the advantages of high yielding candidate protein, thermo-functionally stable, multiplatform expression competent and that elicits neutralizing antibodies. The engineered first generational RBD has an additional N-linked glycosylation site at N532. It was screened and cultured in suitable medium for expression, and further purified from multiple expression platforms. The different platforms were namely, mammalian cells—Expi293F, insect cells—ExpiSf9, and finally the down-selected version pInCV02R in Pichia X-33. It was observed that the vaccine candidates produced from various expression platforms were properly folded, have similar melting temperatures (Tₘ's), bind similarly to ACE2 receptor and to a known characterized SARS-CoV-1 cross neutralizing antibody CR3022. Particularly, mammalian expressed mInCV02R retained functionality to thermal stress by binding to ACE2. It can be contemplated that vaccine candidates purified from Pichia and insect cells retain functionality upon thermal stress. Guinea pig animal immunizations had produced neutralizing antibodies that compete with ACE2 receptor and functionally block the receptor biding motif of RBD to prevent the productive infection of the virus. The present disclosure is the first of its study to describe the glycan engineered version of the RBD and has the advantages of high yielding candidate protein, thermo-functionally stable, multiplatform expression competent and that elicits neutralizing antibodies.

The present disclosure also discloses intermolecular disulfide-linked, trimeric RBD derivative immunogenic composition. In guinea pigs and mice, this immunogenic composition elicits 25-250 fold higher serum neutralizing antibody titers relative to human convalescent sera, with only a three-fold reduction in neutralization against virus containing the B.1.351 RBD mutations. The immunogenic composition protects hamsters from high-dose viral challenge, suggesting it is a good vaccine candidate for future clinical development and deployment, without requiring a cold-chain.

The present disclosure also discloses polypeptide having one or more mutations that elicits high titers of neutralizing antibodies and are highly thermostable with positive Delta-Tₘ. Moreover, the present disclosure also discloses that the presence of two polypeptide in an immunogenic composition, makes the immunogenic composition more thermostable. The immunogenic composition is used in form of a vaccine. Overall, the present disclosure provides cheap, efficacious, COVID-19 vaccines that do not require a cold chain and elicit antibodies capable of neutralizing emerging variants of concern (VOC).

1.-32. (canceled)

33. A polypeptide fragment having an amino acid sequence with at least 95% identity to the amino acid sequence selected from the group consisting of SEQ ID NO: 2, and SEQ ID NO: 4.

34. The polypeptide fragment as claimed in claim 33, wherein the polypeptide having an amino acid sequence selected from the group consisting of SEQ ID NO: 2, SEQ ID NO: 4, SEQ ID NO: 8, or SEQ ID NO: 10.

35. The polypeptide fragment as claimed in claim 33, comprising:
(a) a polypeptide having an amino acid sequence selected from the group consisting of SEQ ID NO: 2; or SEQ ID NO: 8;
(b) a polypeptide having a substitution at an amino acid position in SEQ ID NO: 2, wherein the substitution at the amino acid position is selected from the group consisting of positions at 3, 7, 16, 18, 24, 28, 35, 37, 39, 42, 43, 53, 55, 59, 60, 62, 78, 84, 98, 100, 104, 129, 130, 134, 138, 147, 190, and 197;
(c) a polypeptide having a substitution at an amino acid position in SEQ ID NO: 8, wherein the substitution at the amino acid position is selected from the group consisting of positions at 6, 10, 19, 21, 27, 31, 38, 40, 42, 45, 46, 56, 58, 62, 63, 65, 81, 87, 101, 103, 107, 132, 133, 137, 141, 150, 193, and 200;
(d) a polypeptide having at least one variation in the amino acid sequence as set forth in SEQ ID NO: 2, wherein the at least one variation is selected from the group consisting of P197R/K198R/K199V/S200P/N202V, P197L/Y35F, P197L/A190G/Y35F, P197L/A190G/Y35F/T3H, P197L/A190G/Y35F/T 3H/T55S, P197L/A190G/Y35F, P197L/A190G/Y35F/T3H/T55S/V173D, A18P/P197L/A190G/Y35F/T3H, A18P/A42M/P197L/A190G/Y35F/T3H, A18P/A42M/T100V/P197L/A190G/Y35F/T3H, Y35W/L60M/N118D/Q163S/C195D, A18P/Y35W/P197L, A18P/V37F/P197L, A18P/Y35W/V37F/P197L, A18P/V37F/P197I, A18P/Y35W/V37F/P197I, N13D/A18P/V37F/P197L, N13D/A18P/Y35W/P197L, I28F/Y35W, I28F/F62W, I28F/I104F, Y35W/Y62W, Y35W/I104F, Y62W/I104F, I28F/Y35W/F62W, I28F/Y35W/I104F, I28F/F62W/I104F, Y35W/F62W/I104F, or I28F/Y35W/F62W/I104F;
(e) a polypeptide having at least one variation in the amino acid sequence as set forth in SEQ ID NO: 8, wherein the at least one variation is selected from the group consisting of P200R/K201R/K202V/S203P/N205V, P200L/Y38F, P200L/A193G/Y38F, P200L/A193G/Y38F/T6H, P200L/A193G/Y38F/T6H/T58S, P200L/A193G/Y38F/T6H/T58S/V176D, A21P/P200L/A193G/Y38F/T6H, A21P/A45M/P200L/A193G/Y38F/T6H, A21P/A45M/T103V/P200L/A193G/Y38F/T6H, Y38W/L63M/N121D/Q166S/C198D, A21P/Y38W/P200L, A21P/V40F/P200L, A21P/Y38W/V40F/P200L, A21P/V40F/P200I, A21P/Y38W/V40F/P200I, N16D/A21P/V40F/P200L, N16D/A21P/Y38W/P200L, I31F/Y37W, I31F/F65W, I31F/I107F, Y38W/Y65W, Y38W/I107F, Y65W/I107F, I31F/Y38W/F65W, I31F/Y38W/I107F, I31F/F65W/I107F, Y38W/F65W/I107F, or I31F/Y38W/F65W/I107F; or
(f) a polypeptide having an amino acid selected from the group having the amino acid sequence as set forth in SEQ ID NO: 76, and SEQ ID NO: 79.

36. The polypeptide fragment as claimed in claim 33 comprising:
(a) a polypeptide having an amino acid sequence selected from the group consisting of SEQ ID NO: 4, or SEQ ID NO: 10;
(b) a polypeptide having a substitution at an amino acid position in SEQ ID NO: 4, wherein the substitution at the amino acid position is selected from the group consisting of positions at 2, 6, 15, 17, 23, 27, 34, 36, 38, 41, 42, 52, 54, 58, 59, 61, 77, 83, 97, 99, 103, 128, 129, 133, 137, 146, 189, and 196;
(c) a polypeptide having a substitution at an amino acid position in SEQ ID NO: 10, wherein the substitution at the amino acid position is selected from the group consisting of positions at 5, 9, 18, 20, 26, 30, 37, 39, 41, 44, 45, 55, 57, 61, 62, 64, 80, 86, 100, 102, 106, 131, 132, 136, 140, 149, 192, and 199;
(d) a polypeptide having at least one variation in the amino acid sequence as set forth in SEQ ID NO: 4, wherein the at least one variation is selected from the group consisting of P196R/K197R/K198V/S199P/N201V, P196L/Y34F, P196L/A189G/Y34F, P196L/A189G/Y34F/T2H, P196L/A189G/Y34F/T2H/T54S, P196L/A189G/Y34F/T2H/T54S/V172D, A17P/P196L/A189G/Y34F/T2H, A17P/A41M/P196L/A189G/Y34F/T2H, A17P/A41M/T99V/P196L/A189G/Y34F/T2H, Y34W/L59M/N117D/Q162S/C194D, A17P/Y34W/P196L, A17P/V36F/P196L, A17P/Y34W/V36F/P196L, A17P/V36F/P196I, A17P/Y34W/V36F/P196I, N12D/A17P/V36F/P196L, N12D/A17P/Y34W/P196L, I27F/Y34W, I27F/F61W, I27F/I103F, Y34W/Y61W, Y34W/I103F, Y61W/I103F, I27F/Y34W/F61W, I27F/Y34W/I103F, I27F/F61W/I102F, Y34W/F61W/I103F, I27F/Y34W/F62W/I103F;
(e) a polypeptide having at least one variation in the amino acid sequence as set forth in SEQ ID NO: 10, wherein the at least one variation is selected from the group consisting of P199R/K200R/K201V/S202P/N204V, P199L/Y37F, P199L/A192G/Y37F, P199L/A192G/Y37F/T5H, P199L/A192G/Y 37F/T5H/T57S, P199L/A192G/Y37F/T5H/T57S/V175D, A20P/P199L/A192G/Y37F/T5H, A20P/A44M/P199L/A192G/Y37F/T5H, A20P/A44M/T102V/P199L/A192G/Y37F/T5H, Y37W/L62M/N120D/Q165S/C197D, A20P/Y37W/P199L, A20P/V39F/P199L, A20P/Y37W/V39F/P199L, A20P/V39F/P199I, A20P/Y37W/V39F/P199I, N15D/A20P/V39F/P199L, N15D/A20P/Y37W/P199L, I30F/Y36W, I30F/F64W, I30F/I106F, Y37W/Y64W, Y37W/I106F, Y64W/I106F, I30F/Y37W/F64W, I30F/Y37W/I106F, I30F/F65W/I106F, Y37W/F64W/I106F, I30F/Y37W/F64W/I106F; or
(f) a polypeptide having an amino acid as set forth in SEQ ID NO: 77.

37. The polypeptide fragment as claimed in claim 33 comprising:
(a) a polypeptide having an amino acid sequence selected from the group consisting of SEQ ID NO:14, SEQ ID NO: 16, SEQ ID NO: 18, SEQ ID NO: 20, and SEQ ID NO: 22; or
(b) a polypeptide having an amino acid sequence selected from the group consisting of SEQ ID NO: 81, and SEQ ID NO: 83.

38. The polypeptide fragment as claimed in claim 33 comprising:
(a) a polypeptide having an amino acid sequence selected from the group consisting of SEQ ID NO: 56, SEQ ID NO: 58, and SEQ ID NO: 60;
(b) a polypeptide having an amino acid sequence selected from the group consisting of SEQ ID NO: 52, SEQ ID NO: 54, SEQ ID NO: 62, SEQ ID NO: 64, SEQ ID NO: 66, and SEQ ID NO: 68; or
(c) a polypeptide having an amino acid sequence selected from the group consisting of SEQ ID NO: 24, SEQ ID NO: 26, SEQ ID NO: 28, SEQ ID NO: 30, SEQ ID NO: 32, SEQ ID NO: 34, SEQ ID NO: 38, SEQ ID NO: 40, SEQ ID NO: 42, SEQ ID NO: 44, SEQ ID NO: 46, SEQ ID NO: 48, and SEQ ID NO: 50.

39. The polypeptide fragment as claimed in claim 33, comprising a polypeptide having an amino acid sequence selected from the group consisting of SEQ ID NO: 8, SEQ ID NO: 10, SEQ ID NO: 14, SEQ ID NO: 69, SEQ ID NO: 70, SEQ ID NO: 71, SEQ ID NO: 73, SEQ ID NO: 74, SEQ ID NO: 76, SEQ ID NO: 77, SEQ ID NO: 79, SEQ ID NO: 81, and SEQ ID NO: 83.

40. A recombinant construct comprising a nucleic acid fragment encoding a polypeptide fragment as claimed in claim 33 operably linked to a promoter.

41. The recombinant construct as claimed in claim 40, wherein the recombinant construct further comprises:
(a) a tpa signal sequence;
(b) histidine tag sequence;
(c) a linker;
(d) HRV3C recognition sequence, and
(e) optionally comprising at least one trimerization domain selected the group consisting of human cartilage matrix protein (hCMP), chicken CMP (cCMP), fish cartilage matrix protein (F1CMP), fish isoform 2 cartilage matrix protein (F2-CMP), leucine Zipper with double cysteine (CCIZ), Synthetic trimerization domain (cCMP-IZₘ), foldon, or glycosylated leucine zipper sequence (Gly IZ).

42. The recombinant construct as claimed in claim 41, wherein human cartilage matrix protein (hCMP) having an amino acid sequence as set forth in SEQ ID NO: 87, foldon having an amino acid sequence as set forth in SEQ ID NO: 88, chicken CMP (cCMP) having an amino acid sequence as set forth in SEQ ID NO: 89, fish cartilage matrix protein (F1CMP) having an amino acid sequence as set forth in SEQ ID NO: 90, fish isoform 2 cartilage matrix protein (F2-CMP) having an amino acid sequence as set forth in SEQ ID NO: 91, leucine Zipper with double cysteine (CCIZ) having an amino acid sequence as set forth in SEQ ID NO: 92, synthetic trimerization domain (cCMP-IZₘ) having an amino acid sequence as set forth in SEQ ID NO: 93, or glycosylated leucine zipper sequence (Gly IZ) having an amino acid sequence as set forth in SEQ ID NO: 94.

43. The recombinant construct as claimed in claim 40, wherein the nucleic acid fragment has a nucleotide sequence selected from the group consisting of SEQ ID NO: 1, SEQ ID NO: 3, SEQ ID NO: 7, SEQ ID NO: 9, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 75, SEQ ID NO: 78, SEQ ID NO: 80, SEQ ID NO: 82.

44. An immunogenic composition comprising the polypeptide fragment as claimed in claim 33, and a pharmaceutically acceptable carrier.

45. The immunogenic composition as claimed in claim 44, comprising:
(a) a combination of at least two polypeptide fragments having an amino acid sequence selected from the group consisting of SEQ ID NO: 69, and SEQ ID No: 78, and a pharmaceutical acceptable carrier;
(b) a combination of at least two polypeptide fragments having an amino acid sequence selected from the group consisting of SEQ ID NO: 81, and SEQ ID NO: 83, and a pharmaceutically acceptable carrier.

46. The immunogenic composition as claimed in claim 44, wherein the pharmaceutically acceptable carrier is selected from the group consisting of at least one adjuvant, and excipients.

47. The immunogenic composition as claimed in claim 46, wherein the adjuvant is selected from the group consisting of an oil-in-water adjuvant, a polymer and water adjuvant, a water-in-oil adjuvant, an aluminum hydroxide adjuvant, and combinations thereof.

48. The immunogenic composition as claimed in claim 44, wherein the immunogenic composition is administered by a method selected from the group consisting of intranasal, subcutaneous, intravenous, intra-arterial, intra-peritoneal, intramuscular, intradermal, oral, dermal, and buccal.

49. The immunogenic composition as claimed in claim 44, wherein immunogenic composition is in form of a vaccine.


SEQUENCE LISTING (101 sequences)

SEQ ID NO: 1 - DNA, 606 bp, Artificial Sequence
  depicts the nucleic acid sequence encoding SARS CoV-2 RBD (331-532).
aatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaat

SEQ ID NO: 2 - protein, 202 aa, Artificial Sequence
  depicts the amino acid sequence of SARS CoV-2 RBD (331-532).
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 3 - DNA, 603 bp, Artificial Sequence
  depicts the nucleic acid sequence of encoding SARS-CoV-2 RBD N-1 (332-532).
attacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaat

SEQ ID NO: 4 - protein, 201 aa, Artificial Sequence
  depicts the amino acid sequence of SARS-CoV-2 RBD N-1 (332-532).
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 5 - DNA, 598 bp, Artificial Sequence
  depicts the nucleic acid sequence encoding SARS CoV-2 RBD (332-530).
attactaatttgtgtccttttggtgaagttttcaacgctactagatttgcttctgtttatgcttggaacagaaagagaatctctaactgtgttgctgattactctgttttgtacaactctgcttctttttctactttcaagtgttatggtgtttctccaactaagttgaacgatttgtgtttcactaacgtttacgctgattctttcgttattagaggagatgaggttagacaaattgctcctggtcaaactggtaaaatcgctgattacaactacaagttgccagatgatttcactggttgtgttattgcttggaactctaacaatttggattctaaggttggtggtaactacaactatttgtacagattgtttagaaagtctaacttgaagcctttcgaaagagatatttctactgagatctaccaagctggttctactccatgtaacggtgttgaaggttttaattgttacttcccattgcaatcttacggttttcaacctactaacggtgttggttatcaaccatacagagttgttgttttgtctttcgaattgttgcatgctccagctactgtttgtggtcctaagaaatctg

SEQ ID NO: 6 - protein, 199 aa, Artificial Sequence
  depicts the amino acid sequence of SARS-CoV-2 RBD 3 (332-530).
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 7 - DNA, 687 bp, Artificial Sequence
  depicts the nucleic acid sequence encoding mInCV01R (SARS CoV-2 RBD).
atggatgcaatgaagagagggctctgctgtgtgctgctgctgtgtggagcagtcttcgtttcgcccagcgagatctccaacatcaccaatctgtgccccttcggcgaggtgtttaacgccacacggttcgccagcgtgtacgcctggaaccggaagagaatctccaattgcgtggccgactattctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggacctaagaagagcacaaattga

SEQ ID NO: 8 - protein, 205 aa, Artificial Sequence
  depicts the amino acid sequence of mInCV01R (SARS CoV-2 RBD) having EIS at the N-terminal
EISNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 9 - DNA, 684 bp, Artificial Sequence
  depicts the nucleic acid sequence encoding mInCV02R (SARS CoV-2 RBD N-1).
atggatgcaatgaagagagggctctgctgtgtgctgctgctgtgtggagcagtcttcgtttcgcccagcgagatctccatcaccaatctgtgccccttcggcgaggtgtttaacgccacacggttcgccagcgtgtacgcctggaaccggaagagaatctccaattgcgtggccgactattctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggacctaagaagagcacaaattga

SEQ ID NO: 10 - protein, 204 aa, Artificial Sequence
  depicts the amino acid sequence of the variant of mInCV02R (SARS CoV-2 RBD N-1) having EIS at the N-terminal.
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 11 - DNA, 678 bp, Artificial Sequence
  depicts the nucleic acid sequence encoding the mInCV22R SARS-CoV-2 RBD 3 (332-530).
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtcctag

SEQ ID NO: 12 - protein, 202 aa, Artificial Sequence
  depicts the amino acid sequence of SARS-CoV-2 RBD 3 (332-530).
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 13 - DNA, 855 bp, Artificial Sequence
  depicts the nucleic acid sequence encoding mInCV21R (SARS CoV-2 hCMP-RBD).
atggatgcaatgaagagagggctctgctgtgtgctgctgctgtgtggagcagtcttcgtttcgcccagcgagatctccgaagaagatccttgcgcctgcgagagcctggtcaagtttcaggccaaagtggaaggcctgctgcaggctctgacaagaaagctggaagccgtgtccaagcggctggccatcctggaaaacacagtggtggctagcagcgagggcaccatgatgagaggcgagctgaagaacatcaccaatctgtgccccttcggcgaggtgtttaacgccacacggttcgccagcgtgtacgcctggaaccggaagagaatctccaattgcgtggccgactattctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggacctaagaagagcacaaattga

SEQ ID NO: 14 - protein, 261 aa, Artificial Sequence
  depicts the amino acid sequence of mInCV21R (SARS CoV-2 hCMP-RBD) having EIS + hCMP at the N-terminal.
EISEEDPCACESLVKFQAKVEGLLQALTRKLEAVSKRLAILENTVVASSEGTMMRGELKNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 15 - DNA, 854 bp, Artificial Sequence
  depicts the nucleic acid sequence encoding mInCV26R (SARS CoV-2 RBD with hCMP at C-term)
tggatgcaatgaagagagggctctgctgtgtgctgctgctgtgtggagcagtcttcgtttcgcccagcgagatctccatcaccaatctgtgccccttcggcgaggtgtttaacgccacacggttcgccagcgtgtacgcctggaaccggaagagaatctccaattgcgtggccgactattctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggacctaagaagagcacaaatgccggatctggatctggatctggatccgccggatctgaagaagatccttgcgcctgcgagagcctggtcaagtttcaggccaaagtggaaggcctgctgcaggctctgacaagaaagctggaagccgtgtccaagcggctggccatcctggaaaacacagtggtggcctcttag

SEQ ID NO: 16 - protein, 261 aa, Artificial Sequence
  depicts the amino acid sequence of mInCV26R (SARS CoV-2 RBD with hCMP + EIS at C-terminal
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNAGSGSGSGSAGSEEDPCACESLVKFQAKVEGLLQALTRKLEAVSKRLAILENTVVAS

SEQ ID NO: 17 - DNA, 795 bp, Artificial Sequence
  depicts the nucleic acid sequence encoding mInCV27R (SARS CoV-2 RBD with Foldon at N-terminal)
atggatgcaatgaagagagggctctgctgtgtgctgctgctgtgtggagcagtcttcgtttcgcccagcgagatctccggctatatccccgaggctcctagagatggacaggcctatgtgcggaaggatggcgaatgggtgctgctgagcacatttctcggctctggcggctctggcggcggcgctagcatcaccaatctgtgccccttcggcgaggtgtttaacgccacacggttcgccagcgtgtacgcctggaaccggaagagaatctccaattgcgtggccgactattctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggacctaagaagagcacaaattga

SEQ ID NO: 18 - protein, 241 aa, Artificial Sequence
  depicts the amino acid sequence of mInCV27R (SARS CoV-2 RBD with Foldon + EIS at N terminal)
EISGYIPEAPRDGQAYVRKDGEWVLLSTFLGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 19 - DNA, 834 bp, Artificial Sequence
  depicts the nucleic acid sequence encoding mInCV28R (SARS CoV-2 RBD with GlyIZ at N terminal)
atggatgcaatgaagagagggctctgctgtgtgctgctgctgtgtggagcagtcttcgtttcgcccagcgagatctccaatggcacaggcagaatgaagcagatcgaggacaagatcgagaacatcaccagcaaaatctacaatatcaccaacgagatcgcccggatcaagaagctgatcggcaatagaacagccagcggctctggcggctctggcggcggcgctagcatcaccaatctgtgccccttcggcgaggtgtttaacgccacacggttcgccagcgtgtacgcctggaaccggaagagaatctccaattgcgtggccgactattctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggacctaagaagagcacaaattga

SEQ ID NO: 20 - protein, 254 aa, Artificial Sequence
  depicts the amino acid sequence of mInCV28R (SARS CoV-2 RBD with GlyIZ + EIS at N terminal)
EISNGTGRMKQIEDKIENITSKIYNITNEIARIKKLIGNRTASGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 21 - DNA, 840 bp, Artificial Sequence
  depicts the nucleic acid sequence encoding mInCV29R (SARS CoV-2 RBD with GlyIZ at C-terminal)
atggatgcaatgaagagagggctctgctgtgtgctgctgctgtgtggagcagtcttcgtttcgcccagcgagatctccatcaccaatctgtgccccttcggcgaggtgtttaacgccacacggttcgccagcgtgtacgcctggaaccggaagagaatctccaattgcgtggccgactattctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggacctaagaagagcacaaatgccggatctggatctggatctggatccgccggctctaatggcacaggcagaatgaagcagatcgaggacaagatcgagaacatcaccagcaaaatctacaatatcaccaacgagatcgcccggatcaagaagctgatcggcaatagaacagccagctag

SEQ ID NO: 22 - protein, 256 aa, Artificial Sequence
  depicts the amino acid sequence of mInCV29R (SARS CoV-2 RBD with GlyIZ at C-terminal)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNAGSGSGSGSAGSNGTGRMKQIEDKIENITSKIYNITNEIARIKKLIGNRTAS

SEQ ID NO: 23 - DNA, 1302 bp, Artificial Sequence
  depicts the nucleic acid sequence encoding mInCV42R (SARS CoV-2 RBD Chimera Dimer)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccatcacaaatctgtgtcccttcggcgaggtgttcaatgccacaaaattccctagcgtgtacgcctgggagcggaagaaaatcagcaattgcgtggccgattatagcgtgctgtacaactccacattcttcagcacctttaagtgctacggagtttctgccacaaagctgaatgacctgtgcttcagcaacgtgtacgccgacagcttcgttgtgaagggcgacgacgtgcggcagatcgctcctggccagacaggcgtgatcgccgactacaactacaaactgcccgacgacttcatgggctgtgtgctggcctggaatacccggaacatcgacgccaccagcaccggaaattacaactacaagtaccgcctgttcagaaaaagcaacctgaagcccttcgaacgggacatcagcaccgaaatctaccaagccggcagcaccccttgtaacggtgtggaaggctttaactgctatttccccctgcaatcttacggcttccagcctaccaacggcgtgggctaccagccttacagagtggtcgtcctgtctttcgagctgctgaacgccccagctaccgtgtgtggacctaagctgagcaccgattag

SEQ ID NO: 24 - protein, 410 aa, Artificial Sequence
  depicts the amino acid sequence of mInCV42R (SARS CoV-2 RBD Chimera Dimer)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRLFRKSNLKPFERDISTEIYQAGSTPCNGVEGFNCYFPLQSYGFQPTNGVGYQPYRVVVLSFELLNAPATVCGPKLSTD

SEQ ID NO: 25 - DNA, 1464 bp, Artificial Sequence
  depicts the nucleic acid sequence encoding mInCV30R (SARS CoV-2 RBD Chimera Dimer with GlyIZ at C-terminal)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccatcacaaatctgtgtcccttcggcgaggtgttcaatgccacaaaattccctagcgtgtacgcctgggagcggaagaaaatcagcaattgcgtggccgattatagcgtgctgtacaactccacattcttcagcacctttaagtgctacggagtttctgccacaaagctgaatgacctgtgcttcagcaacgtgtacgccgacagcttcgttgtgaagggcgacgacgtgcggcagatcgctcctggccagacaggcgtgatcgccgactacaactacaaactgcccgacgacttcatgggctgtgtgctggcctggaatacccggaacatcgacgccaccagcaccggaaattacaactacaagtaccgcctgttcagaaaaagcaacctgaagcccttcgaacgggacatcagcaccgaaatctaccaagccggcagcaccccttgtaacggtgtggaaggctttaactgctatttccccctgcaatcttacggcttccagcctaccaacggcgtgggctaccagccttacagagtggtcgtcctgtctttcgagctgctgaacgccccagctaccgtgtgtggacctaagctgagcaccgatggcagcgccggatctggatctggatctggatccgccggctctaatggcacaggcagaatgaagcagatcgaggacaagatcgagaacatcaccagcaaaatctacaatatcaccaacgagatcgcccggatcaagaagctgatcggcaatagaacagccagctag

SEQ ID NO: 26 - protein, 464 aa, Artificial Sequence
  depicts the amino acid sequence of mInCV30R (SARS CoV-2 RBD Chimera Dimer with GlyIZ at the C-terminal)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRLFRKSNLKPFERDISTEIYQAGSTPCNGVEGFNCYFPLQSYGFQPTNGVGYQPYRVVVLSFELLNAPATVCGPKLSTDGSAGSGSGSGSAGSNGTGRMKQIEDKIENITSKIYNITNEIARIKKLIGNRTAS

SEQ ID NO: 27 - DNA, 1452 bp, Artificial Sequence
  depicts the nucleic acid sequence encoding mInCV31R (SARS CoV-2 RBD chimera dimer with GlyIZ at N-terminal)
atggatgcaatgaagagagggctctgctgtgtgctgctgctgtgtggagcagtcttcgtttcgcccagcgagatctccaatggcacaggcagaatgaagcagatcgaggacaagatcgagaacatcaccagcaaaatctacaatatcaccaacgagatcgcccggatcaagaagctgatcggcaatagaacagccagcggctctggcggctctggcggcggcgctagcattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccatcacaaatctgtgtcccttcggcgaggtgttcaatgccacaaaattccctagcgtgtacgcctgggagcggaagaaaatcagcaattgcgtggccgattatagcgtgctgtacaactccacattcttcagcacctttaagtgctacggagtttctgccacaaagctgaatgacctgtgcttcagcaacgtgtacgccgacagcttcgttgtgaagggcgacgacgtgcggcagatcgctcctggccagacaggcgtgatcgccgactacaactacaaactgcccgacgacttcatgggctgtgtgctggcctggaatacccggaacatcgacgccaccagcaccggaaattacaactacaagtaccgcctgttcagaaaaagcaacctgaagcccttcgaacgggacatcagcaccgaaatctaccaagccggcagcaccccttgtaacggtgtggaaggctttaactgctatttccccctgcaatcttacggcttccagcctaccaacggcgtgggctaccagccttacagagtggtcgtcctgtctttcgagctgctgaacgccccagctaccgtgtgtggacctaagctgagcaccgattga

SEQ ID NO: 28 - protein, 460 aa, Artificial Sequence
  depicts the amino acid sequence of mInCV31R (SARS CoV-2 RBD chimera dimer with Gly IZ at N-terminal)
EISNGTGRMKQIEDKIENITSKIYNITNEIARIKKLIGNRTASGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRLFRKSNLKPFERDISTEIYQAGSTPCNGVEGFNCYFPLQSYGFQPTNGVGYQPYRVVVLSFELLNAPATVCGPKLSTD

SEQ ID NO: 29 - DNA, 1425 bp, Artificial Sequence
  depicts the nucleic acid sequence encoding mInCV32R (SARS CoV-2 RBD chimera dimer with Foldon at C-terminal)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccatcacaaatctgtgtcccttcggcgaggtgttcaatgccacaaaattccctagcgtgtacgcctgggagcggaagaaaatcagcaattgcgtggccgattatagcgtgctgtacaactccacattcttcagcacctttaagtgctacggagtttctgccacaaagctgaatgacctgtgcttcagcaacgtgtacgccgacagcttcgttgtgaagggcgacgacgtgcggcagatcgctcctggccagacaggcgtgatcgccgactacaactacaaactgcccgacgacttcatgggctgtgtgctggcctggaatacccggaacatcgacgccaccagcaccggaaattacaactacaagtaccgcctgttcagaaaaagcaacctgaagcccttcgaacgggacatcagcaccgaaatctaccaagccggcagcaccccttgtaacggtgtggaaggctttaactgctatttccccctgcaatcttacggcttccagcctaccaacggcgtgggctaccagccttacagagtggtcgtcctgtctttcgagctgctgaacgccccagctaccgtgtgtggacctaagctgagcaccgatggcagcgccggatctggatctggatctggatccgccggctctggctatatccccgaggctcctagagatggacaggcctatgtgcggaaggatggcgaatgggtgctgctgagcacatttctctag

SEQ ID NO: 30 - protein, 451 aa, Artificial Sequence
  depicts the amino acid sequence of mInCV32R (SARS CoV-2 RBD chimera dimer with Foldon at C-terminal)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRLFRKSNLKPFERDISTEIYQAGSTPCNGVEGFNCYFPLQSYGFQPTNGVGYQPYRVVVLSFELLNAPATVCGPKLSTDGSAGSGSGSGSAGSGYIPEAPRDGQAYVRKDGEWVLLSTFL

SEQ ID NO: 31 - DNA, 1413 bp, Artificial Sequence
  depicts the nucleic acid sequence encoding mInCV33R (SARS CoV-2 RBD chimera dimer with Foldon at N-terminal)
atggatgcaatgaagagagggctctgctgtgtgctgctgctgtgtggagcagtcttcgtttcgcccagcgagatctccggctatatccccgaggctcctagagatggacaggcctatgtgcggaaggatggcgaatgggtgctgctgagcacatttctcggctctggcggctctggcggcggcgctagcattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccatcacaaatctgtgtcccttcggcgaggtgttcaatgccacaaaattccctagcgtgtacgcctgggagcggaagaaaatcagcaattgcgtggccgattatagcgtgctgtacaactccacattcttcagcacctttaagtgctacggagtttctgccacaaagctgaatgacctgtgcttcagcaacgtgtacgccgacagcttcgttgtgaagggcgacgacgtgcggcagatcgctcctggccagacaggcgtgatcgccgactacaactacaaactgcccgacgacttcatgggctgtgtgctggcctggaatacccggaacatcgacgccaccagcaccggaaattacaactacaagtaccgcctgttcagaaaaagcaacctgaagcccttcgaacgggacatcagcaccgaaatctaccaagccggcagcaccccttgtaacggtgtggaaggctttaactgctatttccccctgcaatcttacggcttccagcctaccaacggcgtgggctaccagccttacagagtggtcgtcctgtctttcgagctgctgaacgccccagctaccgtgtgtggacctaagctgagcaccgattga

SEQ ID NO: 32 - protein, 447 aa, Artificial Sequence
  depicts the amino acid sequence of mInCV33R (SARS CoV-2 RBD chimera dimer with Foldon at N-terminal)
EISGYIPEAPRDGQAYVRKDGEWVLLSTFLGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRLFRKSNLKPFERDISTEIYQAGSTPCNGVEGFNCYFPLQSYGFQPTNGVGYQPYRVVVLSFELLNAPATVCGPKLSTD

SEQ ID NO: 33 - DNA, 1479 bp, Artificial Sequence
  depicts the nucleic acid sequence encoding mInCV34R (SARS CoV-2 RBD chimera dimer with hCMP at C terminal)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccatcacaaatctgtgtcccttcggcgaggtgttcaatgccacaaaattccctagcgtgtacgcctgggagcggaagaaaatcagcaattgcgtggccgattatagcgtgctgtacaactccacattcttcagcacctttaagtgctacggagtttctgccacaaagctgaatgacctgtgcttcagcaacgtgtacgccgacagcttcgttgtgaagggcgacgacgtgcggcagatcgctcctggccagacaggcgtgatcgccgactacaactacaaactgcccgacgacttcatgggctgtgtgctggcctggaatacccggaacatcgacgccaccagcaccggaaattacaactacaagtaccgcctgttcagaaaaagcaacctgaagcccttcgaacgggacatcagcaccgaaatctaccaagccggcagcaccccttgtaacggtgtggaaggctttaactgctatttccccctgcaatcttacggcttccagcctaccaacggcgtgggctaccagccttacagagtggtcgtcctgtctttcgagctgctgaacgccccagctaccgtgtgtggacctaagctgagcaccgatggcagcgccggatctggatctggatctggatccgccggctctgaagaagatccttgcgcctgcgagagcctggtcaagtttcaggccaaagtggaaggcctgctgcaggctctgacaagaaagctggaagccgtgtccaagcggctggccatcctggaaaacacagtggtggcctcttag

SEQ ID NO: 34 - protein, 469 aa, Artificial Sequence
  depicts the amino acid sequence of mInCV34R (SARS CoV-2 RBD chimera dimer with hCMP at C-terminal)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRLFRKSNLKPFERDISTEIYQAGSTPCNGVEGFNCYFPLQSYGFQPTNGVGYQPYRVVVLSFELLNAPATVCGPKLSTDGSAGSGSGSGSAGSEEDPCACESLVKFQAKVEGLLQALTRKLEAVSKRLAILENTVVAS

SEQ ID NO: 35 - DNA, 1467 bp, Artificial Sequence
  depicts the nucleic acid sequence encoding mInCV35R (SARS CoV-2 RBD chimera dimer with hCMP at N-terminal)
atggatgcaatgaagagagggctctgctgtgtgctgctgctgtgtggagcagtcttcgtttcgcccagcgagatctccgaagaagatccttgcgcctgcgagagcctggtcaagtttcaggccaaagtggaaggcctgctgcaggctctgacaagaaagctggaagccgtgtccaagcggctggccatcctggaaaacacagtggtggcctctggctctggcggctctggcggcggcgctagcattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccatcacaaatctgtgtcccttcggcgaggtgttcaatgccacaaaattccctagcgtgtacgcctgggagcggaagaaaatcagcaattgcgtggccgattatagcgtgctgtacaactccacattcttcagcacctttaagtgctacggagtttctgccacaaagctgaatgacctgtgcttcagcaacgtgtacgccgacagcttcgttgtgaagggcgacgacgtgcggcagatcgctcctggccagacaggcgtgatcgccgactacaactacaaactgcccgacgacttcatgggctgtgtgctggcctggaatacccggaacatcgacgccaccagcaccggaaattacaactacaagtaccgcctgttcagaaaaagcaacctgaagcccttcgaacgggacatcagcaccgaaatctaccaagccggcagcaccccttgtaacggtgtggaaggctttaactgctatttccccctgcaatcttacggcttccagcctaccaacggcgtgggctaccagccttacagagtggtcgtcctgtctttcgagctgctgaacgccccagctaccgtgtgtggacctaagctgagcaccgattga

SEQ ID NO: 36 - protein, 465 aa, Artificial Sequence
  depicts the amino acid sequence of mInCV35R (SARS CoV-2 RBD chimera dimer with hCMP at N-terminal)
EISEEDPCACESLVKFQAKVEGLLQALTRKLEAVSKRLAILENTVVASGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRLFRKSNLKPFERDISTEIYQAGSTPCNGVEGFNCYFPLQSYGFQPTNGVGYQPYRVVVLSFELLNAPATVCGPKLSTD

SEQ ID NO: 37 - DNA, 1458 bp, Artificial Sequence
  depicts the nucleic acid sequence encoding mInCV36R (SARS CoV-2 RBD dimer with GlyIZ at C-terminal)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaacgccggatctggatctggatctggatccgccggctctaatggcacaggcagaatgaagcagatcgaggacaagatcgagaacatcaccagcaaaatctacaatatcaccaacgagatcgcccggatcaagaagctgatcggcaatagaacagccagctag

SEQ ID NO: 38 - protein, 462 aa, Artificial Sequence
  depicts the amino acid sequence of mInCV36R (SARS CoV-2 RBD dimer with GlyIZ at C-terminal)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNAGSGSGSGSAGSNGTGRMKQIEDKIENITSKIYNITNEIARIKKLIGNRTAS

SEQ ID NO: 39 - DNA, 1452 bp, Artificial Sequence
  depicts the nucleic acid sequence encoding mInCV37R (SARS CoV-2 RBD dimer with Gly IZ at N-terminal)
atggatgcaatgaagagagggctctgctgtgtgctgctgctgtgtggagcagtcttcgtttcgcccagcgagatctccaatggcacaggcagaatgaagcagatcgaggacaagatcgagaacatcaccagcaaaatctacaatatcaccaacgagatcgcccggatcaagaagctgatcggcaatagaacagccagcggctctggcggctctggcggcggcgctagcattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactga

SEQ ID NO: 40 - protein, 460 aa, Artificial Sequence
  depicts the amino acid sequence of mInCV37R (SARS CoV-2 RBD dimer with Gly IZ at N-terminal)
EISNGTGRMKQIEDKIENITSKIYNITNEIARIKKLIGNRTASGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 41 - DNA, 1419 bp, Artificial Sequence
  depicts the nucleic acid sequence encoding mInCV38R (SARS CoV-2 RBD dimer with Foldon at C terminal)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaacgccggatctggatctggatctggatccgccggctctggctatatccccgaggctcctagagatggacaggcctatgtgcggaaggatggcgaatgggtgctgctgagcacatttctctag

SEQ ID NO: 42 - protein, 449 aa, Artificial Sequence
  depicts the amino acid sequence of mInCV38R (SARS CoV-2 RBD dimer with Foldon at C-terminal)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNAGSGSGSGSAGSGYIPEAPRDGQAYVRKDGEWVLLSTFL

SEQ ID NO: 43 - DNA, 1413 bp, Artificial Sequence
  depicts the nucleic acid sequence encoding mInCV39R (SARS CoV-2 RBD dimer Foldon at N-terminal)
atggatgcaatgaagagagggctctgctgtgtgctgctgctgtgtggagcagtcttcgtttcgcccagcgagatctccggctatatccccgaggctcctagagatggacaggcctatgtgcggaaggatggcgaatgggtgctgctgagcacatttctcggctctggcggctctggcggcggcgctagcattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactga

SEQ ID NO: 44 - protein, 447 aa, Artificial Sequence
  depicts the amino acid sequence of mInCV39R (SARS CoV-2 RBD dimer with Foldon at N-terminal)
EISGYIPEAPRDGQAYVRKDGEWVLLSTFLGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 45 - DNA, 1473 bp, Artificial Sequence
  depicts the nucleic acid sequence encoding mInCV40R (SARS CoV-2 RBD dimer with hCMP at C-terminal)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaacgccggatctggatctggatctggatccgccggctctgaagaagatccttgcgcctgcgagagcctggtcaagtttcaggccaaagtggaaggcctgctgcaggctctgacaagaaagctggaagccgtgtccaagcggctggccatcctggaaaacacagtggtggcctcttag

SEQ ID NO: 46 - protein, 467 aa, Artificial Sequence
  depicts the amino acid sequence of mInCV40R (SARS CoV-2 RBD dimer with hCMP at C-terminal)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNAGSGSGSGSAGSEEDPCACESLVKFQAKVEGLLQALTRKLEAVSKRLAILENTVVAS

SEQ ID NO: 47 - DNA, 1467 bp, Artificial Sequence
  depicts the nucleic acid sequence encoding mInCV41R (SARS CoV-2 RBD dimer with hCMP at N-terminal)
atggatgcaatgaagagagggctctgctgtgtgctgctgctgtgtggagcagtcttcgtttcgcccagcgagatctccgaagaagatccttgcgcctgcgagagcctggtcaagtttcaggccaaagtggaaggcctgctgcaggctctgacaagaaagctggaagccgtgtccaagcggctggccatcctggaaaacacagtggtggcctctggctctggcggctctggcggcggcgctagcattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactga

SEQ ID NO: 48 - protein, 465 aa, Artificial Sequence
  depicts the amino acid sequence of mInCV41R (SARS CoV-2 RBD dimer with hCMP at N-terminal)
EISEEDPCACESLVKFQAKVEGLLQALTRKLEAVSKRLAILENTVVASGSGGSGGGASITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 49 - DNA, 1302 bp, Artificial Sequence
  depicts the nucleic acid sequence encoding mInCV43R (SARS CoV-2 RBD dimer)
atggatgcaatgaagagagggctctgctgtgtgctgctgctgtgtggagcagtcttcgtttcgcccagcgagatctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactctggcgccggctccattaccaacctgtgcccttttggcgaagtgttcaatgccaccaggttcgccagtgtgtatgcttggaacagaaagagaatctctaactgcgtggccgactacagcgtgctgtacaacagcgccagcttctccaccttcaagtgctacggcgtgtccccaaccaaactgaacgacctgtgttttaccaacgtgtacgccgactcctttgtgatccggggcgacgaggtcagacagatcgctcccggacagaccggcaagatcgccgattacaactacaagctgcctgatgatttcaccggctgcgtgatcgcatggaacagcaacaaccttgatagcaaggtgggcggcaactacaactacctgtacagactgtttagaaagagcaacctcaagcctttcgagagagatatctccacagaaatctaccaggccggatctacaccttgcaacggagtggaaggcttcaactgctacttcccactgcagagctacggcttccagcctacaaacggcgtgggatatcagccctacagagtggtggtgctgagctttgagctcctgcacgcccctgctacagtgtgcggccctaagaagtccaccaactag

SEQ ID NO: 50 - protein, 410 aa, Artificial Sequence
  depicts the amino acid sequence of mInCV43R (SARS CoV-2 RBD dimer)
EISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNSGAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 51 - DNA, 849 bp, Artificial Sequence
  depicts the nucleic acid sequence encoding SARS CoV-2 NTD
gcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaa

SEQ ID NO: 52 - protein, 489 aa, Artificial Sequence
  depicts the amino acid sequence of SARS CoV-2 NTD.
AYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEGSAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 53 - DNA, 1452 bp, Artificial Sequence
  depicts the nucleic acid sequence encoding a fusion polypeptide SARS CoV-2 NTD-RBD (without the linker).
gcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaat

SEQ ID NO: 54 - protein, 489 aa, Artificial Sequence; identical to SEQ ID NO: 52
  depicts the amino acid sequence of polypeptide SARS CoV-2 NTD-RBD (with a linker GSAGS).

SEQ ID NO: 55 - DNA, 732 bp, Artificial Sequence
  depicts the nucleic acid sequence encoding iInCV01R (SARS CoV-2 RBD)
atgctgctggtgaaccagtcacaccagggcttcaacaaggagcacacctcaaagatggtctccgccatcgtcctgtacgtgctgctcgctgctgctgctcactctgctttcgccgagatctctaacatcaccaacctgtgccctttcggagaagtgttcaacgctactaggttcgcctccgtctacgcttggaaccgcaagcgtatctcaaactgcgtcgccgactactccgtgctgtacaactccgctagcttctctactttcaagtgctacggagtgtcacccaccaagctgaacgacctgtgcttcactaacgtctacgccgactccttcgtgatccgcggtgacgaggtccgtcagatcgctcctggtcagaccggcaagatcgctgactacaactacaagctgcctgacgacttcactggttgcgtgatcgcttggaactcaaacaacctggactccaaggtcggtggcaactacaactacctgtacaggctgttcagaaagagcaacctgaagcccttcgagagggacatcagcaccgaaatctaccaggctggttctactccatgcaacggagtggaaggtttcaactgctacttcccactgcagagctacggcttccagcctaccaacggagtcggttaccagccctacagagtggtcgtgctgtctttcgagctgctccacgctcctgctactgtgtgcggacccaagaagagcactaactaa

SEQ ID NO: 56 - protein, 205 aa, Artificial Sequence; identical to SEQ ID NO: 8
  depicts the amino acid sequence of iInCV01R (SARS CoV-2 RBD)

SEQ ID NO: 57 - DNA, 729 bp, Artificial Sequence
  depicts the nucleic acid sequence encoding iInCV02R (SARS CoV-2 RBD)
atgctgctggtgaaccagtcacaccagggcttcaacaaggagcacacctcaaagatggtctccgccatcgtcctgtacgtgctgctcgctgctgctgctcactctgctttcgccgagatctctatcaccaacctgtgccctttcggagaagtgttcaacgctactaggttcgcctccgtctacgcttggaaccgcaagcgtatctcaaactgcgtcgccgactactccgtgctgtacaactccgctagcttctctactttcaagtgctacggagtgtcacccaccaagctgaacgacctgtgcttcactaacgtctacgccgactccttcgtgatccgcggtgacgaggtccgtcagatcgctcctggtcagaccggcaagatcgctgactacaactacaagctgcctgacgacttcactggttgcgtgatcgcttggaactcaaacaacctggactccaaggtcggtggcaactacaactacctgtacaggctgttcagaaagagcaacctgaagcccttcgagagggacatcagcaccgaaatctaccaggctggttctactccatgcaacggagtggaaggtttcaactgctacttcccactgcagagctacggcttccagcctaccaacggagtcggttaccagccctacagagtggtcgtgctgtctttcgagctgctccacgctcctgctactgtgtgcggacccaagaagagcactaactaa

SEQ ID NO: 58 - protein, 204 aa, Artificial Sequence; identical to SEQ ID NO: 10
  depicts the amino acid sequence of iInCV02R (SARS CoV-2 RBD)

SEQ ID NO: 59 - DNA, 879 bp, Artificial Sequence
  depicts the nucleic acid sequence encoding pInCV02R (SARS CoV-2 RBD N-1 (332-532)
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcattactaatttgtgtccttttggtgaagttttcaacgctactagatttgcttctgtttatgcttggaacagaaagagaatctctaactgtgttgctgattactctgttttgtacaactctgcttctttttctactttcaagtgttatggtgtttctccaactaagttgaacgatttgtgtttcactaacgtttacgctgattctttcgttattagaggagatgaggttagacaaattgctcctggtcaaactggtaaaatcgctgattacaactacaagttgccagatgatttcactggttgtgttattgcttggaactctaacaatttggattctaaggttggtggtaactacaactatttgtacagattgtttagaaagtctaacttgaagcctttcgaaagagatatttctactgagatctaccaagctggttctactccatgtaacggtgttgaaggttttaattgttacttcccattgcaatcttacggttttcaacctactaacggtgttggttatcaaccatacagagttgttgttttgtctttcgaattgttgcatgctccagctactgtttgtggtcctaagaaatctactaattaa

SEQ ID NO: 60 - protein, 207 aa, Artificial Sequence
  depicts the amino acid sequence of pInCV02R (SARS CoV-2 RBD N-1 (332-532).
EAEAEFITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 61 - DNA, 1548 bp, Artificial Sequence
  depicts the nucleic acid sequence encoding mInCV05NR (SARS CoV-2 NTD-RBD)
atggatgcaatgaagagagggctctgctgtgtgctgctgctgtgtggagcagtcttcgtttcgcccagcgagatctccgcctataccaacagcttcacacggggcgtgtactatcccgacaaggtgtttagaagctccgtgctgcactccacccaggatctgttcctgcctttcttttctaacgtgacatggtttcacgccatccacgtgtccggcaccaatggcacaaagaggttcgacaaccccgtgctgcctttcaatgatggcgtgtactttgccagcaccgagaagtccaatatcatccgcggctggatctttggcaccacactggactctaagacccagagcctgctgatcgtgaacaatgccacaaacgtggtcatcaaggtgtgcgagttccagttttgtaatgatcccttcctgggcgtgtactatcacaagaacaataagtcctggatggagtctgagtttcgggtgtattctagcgccaacaattgcaccttcgagtacgtgtctcagccttttctgatggacctggagggcaagcagggcaacttcaagaatctgcgggagttcgtgtttaagaacatcgatggctacttcaaaatctacagcaagcacacacccatcaatctggtgagagacctgcctcagggcttctccgccctggagccactggtggatctgcccatcggcatcaacatcaccaggtttcagacactgctggccctgcaccgctcctatctgaccccaggcgactcctctagcggatggacagcaggagctgccgcctactatgtgggctatctgcagccccggaccttcctgctgaagtacaacgagaatggcaccatcacagacgcagtggattgcgccctggaccccgagagcgagaccaagtgtacactgaagtccttcaccgtggagggctccgccggctctatcacaaacctgtgcccattcggcgaggtgtttaatgccaccaggtttgccagcgtgtacgcctggaaccggaagagaatctccaattgcgtggccgactattctgtgctgtacaacagcgccagcttcagcaccttcaagtgctatggcgtgtctccaaccaagctgaacgacctgtgcttcacaaacgtgtacgccgacagctttgtgatccggggcgatgaggtgagacagatcgcaccaggacagaccggcaagatcgcagactacaactataagctgcccgacgatttcacaggctgcgtgatcgcctggaacagcaacaatctggatagcaaagtgggcggcaactacaattatctgtacaggctgttccgcaagagcaacctgaagccttttgagagggacatctctaccgaaatctaccaggccggcagcacaccatgcaacggcgtggagggcttcaattgttattttccactgcagtcctacggcttccagcccaccaacggcgtgggctatcagccttaccgcgtggtggtgctgtcttttgagctgctgcacgcacctgcaaccgtgtgcggaccaaagaagagcacaaattga

SEQ ID NO: 62 - protein, 492 aa, Artificial Sequence
  depicts the amino acid sequence of mInCV05NR (SARS CoV-2 NTD-RBD)
EISAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPESETKCTLKSFTVEGSAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 63 - DNA, 930 bp, Artificial Sequence
  depicts the nucleic acid sequence encoding mInCV07N (SARS CoV-2 NTD)
atggatgcaatgaagagagggctctgctgtgtgctgctgctgtgtggagcagtcttcgtttcgcccagcgagatctccgcctataccaacagcttcacacggggcgtgtactatcccgacaaggtgtttagaagctccgtgctgcactccacccaggatctgttcctgcctttcttttctaacgtgacatggtttcacgccatccacgtgtccggcaccaatggcacaaagaggttcgacaaccccgtgctgcctttcaatgatggcgtgtactttgccagcaccgagaagtccaatatcatccgcggctggatctttggcaccacactggactctaagacccagagcctgctgatcgtgaacaatgccacaaacgtggtcatcaaggtgtgcgagttccagttttgtaatgatcccttcctgggcgtgtactatcacaagaacaataagtcctggatggagtctgagtttcgggtgtattctagcgccaacaattgcaccttcgagtacgtgtctcagccttttctgatggacctggagggcaagcagggcaacttcaagaatctgcgggagttcgtgtttaagaacatcgatggctacttcaaaatctacagcaagcacacacccatcaatctggtgagagacctgcctcagggcttctccgccctggagccactggtggatctgcccatcggcatcaacatcaccaggtttcagacactgctggccctgcaccgctcctatctgaccccaggcgactcctctagcggatggacagcaggagctgccgcctactatgtgggctatctgcagccccggaccttcctgctgaagtacaacgagaatggcaccatcacagacgcagtggattgcgccctggaccccgagagcgagaccaagtgtacactgaagtccttcaccgtggagtga

SEQ ID NO: 64 - protein, 286 aa, Artificial Sequence
  depicts the amino acid sequence of mInCV07N (SARS CoV-2 NTD)
EISAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPESETKCTLKSFTVE

SEQ ID NO: 65 - DNA, 1743 bp, Artificial Sequence
  depicts the nucleic acid sequence encoding pInCV04NR (SARS CoV-2 NTD-RBD)
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgcttacactaactcttttactagaggtgtttactaccctgataaggtttttagatcctctgttttgcattctactcaagatttgtttttgccatttttctctaacgttacttggttccatgctattcacgtttctggtactaatggtactaagagatttgataacccagttttgccttttaatgatggtgtttacttcgcttctactgagaagtctaacatcatcagaggttggatcttcggtactactttggattctaagactcaatctttgttgatcgttaacaacgctactaacgttgttattaaggtttgtgaatttcaattctgtaacgatccatttttgggtgtttactatcacaagaacaacaagtcttggatggaatctgagttcagagtttattcttctgctaacaactgtactttcgaatacgtttctcaaccattcttgatggatttggagggtaaacagggtaacttcaagaatttgagagaatttgttttcaaaaacatcgatggttacttcaaaatctactctaagcatactccaattaatttggttagagatttgcctcaaggtttttctgctttggagccattggttgatttgcctatcggtattaacatcactagattccaaactttgttggctttgcacagatcctatttgactcctggagattcttcttctggttggactgctggtgctgctgcttactatgttggttatttgcaaccaagaactttcttgttgaagtacaacgaaaacggtactatcactgatgctgttgattgtgctttggacccagaatctgagactaagtgtactttgaaatcttttactgttgagggttctgctggttctattactaacttgtgtccatttggtgaagttttcaatgctactagattcgcttctgtttacgcttggaacagaaagagaatttctaactgtgttgctgattactctgttttgtacaactctgcttctttttctactttcaagtgttatggtgtttctcctactaaattgaacgatttgtgtttcactaacgtttacgctgattctttcgttattagaggagatgaggttagacaaattgctcctggtcaaactggtaaaatcgctgattacaactacaaattgccagatgatttcactggttgtgttattgcttggaactctaacaatttggattctaaggttggtggtaactacaactatttgtacagattgtttagaaagtctaatttgaagcctttcgaaagagatatttctactgaaatctaccaagctggttctactccatgtaacggtgttgaaggttttaattgttacttcccattgcaatcttacggttttcaacctactaacggtgttggttatcaaccatacagagttgttgttttgtctttcgagttgttgcatgctccagctactgtttgtggtcctaagaaatctactaattaa

SEQ ID NO: 66 - protein, 495 aa, Artificial Sequence
  depicts the amino acid sequence of pInCV04NR (SARS CoV-2 NTD-RBD)
EAEAEFAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPESETKCTLKSFTVEGSAGSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 67 - DNA, 1593 bp, Artificial Sequence
  depicts the nucleic acid sequence encoding iInCV03NR (SARS CoV-2 NTD-RBD)
atgctgctggtgaaccagtcccaccagggtttcaacaaggaacacacctccaagatggtcagcgctatcgtcctgtacgtgctgctggctgccgctgcccacagcgccttcgctgagatcagcgcctacaccaactcattcactcgcggcgtgtactaccctgacaaggtcttccgttccagcgtgctgcactccacccaggacctgttcctgcccttcttcagcaacgtgacttggttccacgctatccacgtctccggcaccaacggcactaagaggttcgacaaccctgtgctgcccttcaacgacggagtctacttcgcctctactgaaaagtcaaacatcatcagaggctggatcttcggaaccactctggactccaagacccagagcctgctgatcgtgaacaacgctactaacgtggtcatcaaggtctgcgagttccagttctgcaacgaccctttcctgggtgtgtactaccacaagaacaacaagtcttggatggagtcagagttcagggtgtactcttcagccaacaactgcaccttcgagtacgtctctcagcccttcctgatggacctggaaggaaagcagggtaacttcaagaacctgcgcgagttcgtcttcaagaacatcgacggctacttcaagatttactctaagcacactcctatcaacctggtgcgtgacctgccccagggcttctcagctctggaaccactggtcgacctgcctatcggaatcaacatcaccaggttccagactctgctggctctgcacagatcatacctgaccccaggagactccagctctggttggactgctggtgctgccgcttactacgtgggttacctgcagcctcgcaccttcctgctgaagtacaacgagaacggcaccatcacagacgctgtggactgcgctctggaccccgagtccgaaaccaagtgcactctgaagagcttcaccgtcgaaggctctgccggatcaatcactaacctgtgcccattcggagaggtgttcaacgccacccgtttcgcttctgtctacgcctggaaccgcaagcgtatctctaactgcgtcgctgactactcagtgctgtacaactcagcctccttcagcactttcaagtgctacggcgtgtccccaaccaagctgaacgacctgtgcttcactaacgtctacgctgacagcttcgtgatccgcggagacgaagtccgtcagatcgctcctggtcagaccggcaagatcgccgactacaactacaagctgcctgacgacttcactggctgcgtgatcgcctggaactccaacaacctggacagcaaggtcggtggcaactacaactacctgtacaggctgttcagaaagtccaacctgaagcccttcgagagggacatctctaccgaaatctaccaggctggatcaactccatgcaacggagtggagggtttcaactgctacttcccactgcagtcttacggtttccagcctaccaacggagtcggttaccagccctacagagtggtcgtgctgtcattcgaactgctccacgctcctgctactgtgtgcggaccaaagaagtccactaactaa

SEQ ID NO: 68 - protein, 492 aa, Artificial Sequence; identical to SEQ ID NO: 62
  depicts the amino acid sequence of iInCV03NR (SARS CoV-2 NTD-RBD)

SEQ ID NO: 69 - protein, 202 aa, Artificial Sequence
  depicts the amino acid sequence of DM37
NITNLCPFGEVFNATRFPSVYAWNRKRISNCVADWSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKSTN

SEQ ID NO: 70 - protein, 199 aa, Artificial Sequence
  depicts the amino acid sequence of DM47
ITNLCPFGEVFDATRFPSVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKS

SEQ ID NO: 71 - protein, 199 aa, Artificial Sequence
  depicts the amino acid sequence of DM48
ITNLCPFGEVFDATRFPSVYAWNRKRISNCVADWSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKS

SEQ ID NO: 72 - protein, 199 aa, Artificial Sequence; identical to SEQ ID NO: 71
  depicts the amino acid sequence of pDM48R

SEQ ID NO: 73 - protein, 199 aa, Artificial Sequence
  depicts the amino acid sequence of pDM49R
ITNLCPFGEVFNATRFPSVYAWNRKRISNCVADWSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKS

SEQ ID NO: 74 - protein, 199 aa, Artificial Sequence
  depicts the amino acid sequence of pDM49+SA Mutation
ITNLCPFGEVFNATRFPSVYAWNRKRISNCVADWSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGLKKS

SEQ ID NO: 75 - DNA, 615 bp, Artificial Sequence
  depicts the nucleic acid sequence encoding DM37-CHO
gagatctccaacatcaccaatctgtgccccttcggcgaggtgtttaacgccacacggttcccgagcgtgtacgcctggaaccggaagagaatctccaattgcgtggccgactggtctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggactgaagaagagcacaaat

SEQ ID NO: 76 - protein, 205 aa, Artificial Sequence
  depicts the amino acid sequence of DM37-CHO
EISNITNLCPFGEVFNATRFPSVYAWNRKRISNCVADWSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKSTN

SEQ ID NO: 77 - protein, 201 aa, Artificial Sequence
  depicts the amino acid sequence of DM-37a
ITNLCPFGEVFNATRFPSVYAWNRKRISNCVADWSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKSTN

SEQ ID NO: 78 - DNA, 615 bp, Artificial Sequence
  Depicts the nucleic acid sequence encoding DM37-SA
gagatctccaacatcaccaatctgtgccccttcggcgaggtgtttaacgccacacggttcccgagcgtgtacgcctggaaccggaagagaatctccaattgcgtggccgactggtctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaacatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtgaagggcttcaattgttattttccactgcagtcctacggcttccagcccacatacggcgtgggctatcagccttaccgcgtggtggtgctgtcttttgagctgctgcacgcaccagcaaccgtgtgcggactgaagaagagcacaaat

SEQ ID NO: 79 - protein, 205 aa, Artificial Sequence
  Depicts the amino acid sequence of DM37-SA
EISNITNLCPFGEVFNATRFPSVYAWNRKRISNCVADWSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGLKKSTN

SEQ ID NO: 80 - DNA, 807 bp, Artificial Sequence
  Depicts the nucleic acid sequence encoding hCMP-DM37
gagatctccgaagaagatccttgcgcctgcgagagcctggtcaagtttcaggccaaagtggaaggcctgctgcaggctctgacaagaaagctggaagccgtgtccaagcggctggccatcctggaaaacacagtggtggctagcagcgagggcaccatgatgagaggcgagctgaagaacatcaccaatctgtgccccttcggcgaggtgtttaacgccacacggttcccgagcgtgtacgcctggaaccggaagagaatctccaattgcgtggccgactggtctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggactgaagaagagcacaaatggctccctggaggtgctgttccag

SEQ ID NO: 81 - protein, 269 aa, Artificial Sequence
  depicts the amino acid sequence of hCMP-DM37
EISEEDPCACESLVKFQAKVEGLLQALTRKLEAVSKRLAILENTVVASSEGTMMRGELKNITNLCPFGEVFNATRFPSVYAWNRKRISNCVADWSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKSTNGSLEVLFQ

SEQ ID NO: 82 - DNA, 852 bp, Artificial Sequence
  depicts the nucleic acid sequence encoding hCMP-DM37SA
gagatctccgaagaagatccttgcgcctgcgagagcctggtcaagtttcaggccaaagtggaaggcctgctgcaggctctgacaagaaagctggaagccgtgtccaagcggctggccatcctggaaaacacagtggtggctagcagcgagggcaccatgatgagaggcgagctgaagaacatcaccaatctgtgccccttcggcgaggtgtttaacgccacacggttcccgagcgtgtacgcctggaaccggaagagaatctccaattgcgtggccgactggtctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaacatcgcagactacaactataagctgcccgacgacttcaccggctgcgtgatcgcctggaattctaacaatctggatagcaaagtgggcggcaactacaattatcggtacaggctgttccgcaagagcaacctgaagccttttgagagggacatcagcaccgaaatctaccaggccggctccacaccatgcaacggcgtgaagggcttcaattgttattttccactgcagtcctacggcttccagcccacatacggcgtgggctatcagccttaccgcgtggtggtgctgtcttttgagctgctgcacgcaccagcaaccgtgtgcggactgaagaagagcacaaatggctccctggaggtgctgttccagggaccaggatcccatcatcaccatcatcaccaccaccaccactga

SEQ ID NO: 83 - protein, 269 aa, Artificial Sequence
  depicts the amino acid sequence of hCMP-DM37SA
EISEEDPCACESLVKFQAKVEGLLQALTRKLEAVSKRLAILENTVVASSEGTMMRGELKNITNLCPFGEVFNATRFPSVYAWNRKRISNCVADWSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGLKKSTNGSLEVLFQ

SEQ ID NO: 84 - DNA, 675 bp, Artificial Sequence
  Depicts the nucleic acid sequence encoding mDM46
gagatctccatcaccaatctgtgccccttcggcgaggtgtttaacgccacacggttcccgagcgtgtacgcctggaaccggaagagaatctccaattgcgtggccgactggtctgtgctgtacaatagcgcctccttctctacctttaagtgctatggcgtgtctcctaccaagctgaacgacctgtgcttcacaaacgtgtacgccgacagctttgtgatccggggcgatgaggtgagacagatcgcaccaggacagaccggcaagatcgcagactacaactataagctgcccgacgacttcaccggctgcgtgatcgcctggaattctaacaatctggatagcaaagtgggcggcaactacaattatctgtacaggctgttccgcaagagcaacctgaagccttttgagagggacatcagcaccgaaatctaccaggccggctccacaccatgcaacggcgtggagggcttcaattgttattttccactgcagtcctacggcttccagcccacaaacggcgtgggctatcagccttaccgcgtggtggtgctgtcttttgagctgctgcacgcaccagcaaccgtgtgcggactgaagaagagcggctccctggaggtgctgttccagggaccaggatcccatcatcaccatcatcaccaccaccaccactga

SEQ ID NO: 85 - protein, 202 aa, Artificial Sequence
  depicts the amino acid sequence of mDM46
EISITNLCPFGEVFNATRFPSVYAWNRKRISNCVADWSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGLKKS

SEQ ID NO: 86 - protein, 267 aa, Artificial Sequence
  depicts the amino acid sequence of full length (327-527)
MDAMKRGLCCVLLLCGAVFVSPSEISRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSLEVLFQGPGSHHHHHHHHHH

SEQ ID NO: 87 - protein, 57 aa, Artificial Sequence
  depicts the amino acid sequence of hCMP
EEDPCACESLVKFQAKVEGLLQALTRKLEAVSKRLAILENTVVASSEGTMMRGELKN

SEQ ID NO: 88 - protein, 27 aa, Artificial Sequence
  depicts the amino acid sequence of foldon
GYIPEAPRDGQAYVRKDGEWVLLSTFL

SEQ ID NO: 89 - protein, 42 aa, Artificial Sequence
  depicts the amino acid sequence of Chicken cartilage matrix protein (cCMP)
EDPCECKSIVKFQTKVEELINTLQQKLEAVAKRIEALENKII

SEQ ID NO: 90 - protein, 42 aa, Artificial Sequence
  KDPCACDSLVEFQQATMTTLEQLTRKLAGMTARLEDLENQLL
KDPCACDSLVEFQQATMTTLEQLTRKLAGMTARLEDLENQLL

SEQ ID NO: 91 - protein, 42 aa, Artificial Sequence
  depicts the amino acid sequence of Fish isoform 2 cartilage matrix protein (F2-CMP)
KDPCACESLVEFQQVTMSTLDQLNQKLSAMTKRLEVLESQLL

SEQ ID NO: 92 - protein, 28 aa, Artificial Sequence
  depicts amino acid sequence of Leucine Zipper with double cysteine  (CCIZ)
CCGGIKKEIEAIKKEQEAIKKKIEAIEK

SEQ ID NO: 93 - protein, 37 aa, Artificial Sequence
  depicts the amino acid sequence of Synthetic trimerization domain (cCMP-IZm)
EEDPCECKSIKKEIEAIKKEQEAIKKKIEAIEKEIEA

SEQ ID NO: 94 - protein, 40 aa, Artificial Sequence
  depicts the amino acid sequence of Glycosylated leucine zipper sequence (Gly IZ)
NGTGRMKQIEDKIENITSKIYNITNEIARIKKLIGNRTAS

SEQ ID NO: 95 - protein, 250 aa, Artificial Sequence
  depicts the amino acid sequence of sequence of mInCV01R (SARS CoV-2 RBD) having tpa signal sequence at the N-terminal.
MDAMKRGLCCVLLLCGAVFVSPSEISNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSLEVLFQGPGSHHHHHHHHHH

SEQ ID NO: 96 - protein, 249 aa, Artificial Sequence
  depicts the amino acid sequence of mInCV02R (SARS CoV-2 RBD N-1) having tpa signal sequence at the N-terminal
MDAMKRGLCCVLLLCGAVFVSPSEISITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSLEVLFQGPGSHHHHHHHHHH

SEQ ID NO: 97 - DNA, 33 bp, Artificial Sequence
  depicts the nucleotide sequence of the forward primer
tatataagcttctgcagtcaccgtccttagatc

SEQ ID NO: 98 - DNA, 38 bp, Artificial Sequence
  depicts the nucleotide sequence of reverse primer
tatatctcgagtcactggaacagcacctccagggagcc

SEQ ID NO: 99 - DNA, 20 bp, Artificial Sequence
  depicts the nucleotide sequence of 2019-nCoV_N1-Forward primer
gaccccaaaatcagcgaaat

SEQ ID NO: 100 - DNA, 24 bp, Artificial Sequence
  depicts the nucleotide sequence of 2019-nCoV_N1-Reverse primer
tctggttactgccagttgaatctg

SEQ ID NO: 101 - DNA, 24 bp, Artificial Sequence
  depicts the nucleotide sequence of 2019-nCoV_N1 Probe (6-FAM / BHQ-1)
accccgcattacgtttggtggacc